(19) United States
(12) Patent Application Publication
(10) Pub. No.: US 2005/0089878 A1
(43) Pub. Date: Apr. 28, 2005

(54) Method for determining functional sites in a protein

(76) Inventors: Derek A. Debe, Sierra Madre, CA (US); Joseph F. Danzer, Pasadena, CA (US); Lei Xie, San Diego, CA (US)

Correspondence Address: PERKINS COIE LLP, POST OFFICE BOX 1208, SEATTLE, WA, 98111-1208, US

(21) Appl. No.: 10/764,260

(22) Filed: Jan. 22, 2004

Related U.S. Application Data
(60) Provisional application No. 60/447,562, filed on Feb. 14, 2003.

(51) Int. Cl. C12Q 1/68; G06F 19/00; G01N 33/48; G01N 33/50

(52) U.S. Cl. 435/6; 702/19

(57) ABSTRACT

The present invention relates to improved methods for determining functional residues on the surface of a query protein. The claimed methods rely on determining a plurality of functional annotation scores for a query protein and comparing these functional annotation scores to distributions of similar functional annotation scores derived from a plurality of reference proteins. Based upon these comparisons, a putative functional cluster may be annotated as a functional cluster or a non-functional cluster.

### RELATED APPLICATION

This application claims the benefit of U.S. Provisional Application No. 60/447,562, filed Feb. 14, 2003, which is hereby incorporated by reference in its entirety including drawings as fully set forth herein.

### BACKGROUND OF THE INVENTION

Protein surfaces often contain biologically functional sites such as catalytic sites, ligand binding sites, protein-protein recognition sites and protein anchoring sites. The identification and characterization (referred to as annotation) of functional sites allows for the identification of new biochemical pathways and protein mediated interactions as well as supplements the body of science relating to known pathways and systems. More importantly, functional site annotation may also be used for target identification/validation, to rationalize small molecule screening and to guide medicinal chemistry efforts once a small molecule has been successfully screened against a potential drug target.

Many of the current methods for determining functional sites, or equivalently, functional residues or clusters of residues, include primary sequence comparison methods, and structure based comparison methods. Primary sequence comparison methods identify and characterize a putative functional site on the surface of a protein structure of interest, referred throughout as a query structure or query protein, by determining, whether and to what extent, the surface of the query structure contains residues which are evolutionarily significant across homologous sequences. Structure comparison methods identify and characterize a putative functional site on a query protein by determining whether and to what extent the surface of the query protein is topographically similar to known functional sites.

In general, primary sequence comparison methods for determining functional sites employ the following methodology: 1) determine a family of template sequences homologous to the query sequence by running a sequence homology tool such as the various BLAST, Smith-Waterman, FASTA or Hidden Markov Model algorithms on the query sequence using any large sequence database; 2) determine a multiple sequence alignment of the query sequence and the template sequences; and 3) identify putative functional residues as those surface residues which are highly conserved in the multiple sequence alignment. See e.g. Landgraf, R., Xenarios, I., Eisenberg, D., Three Dimensional Cluster Analysis Identifies Interfaces And Functional Residues In Proteins, J. Mol Biol 307(5):1487-502 (2001). See also the Insight software suite from Accelrys, Inc., (San Diego, Calif., http://www.accelrys.com/insight/binding_site_analysis.html#references).

The use of primary sequence comparison methods to annotate functional sites is predicated on two assumptions: 1) that functional residues are highly and uniformly conserved across homologous sequences; and 2) that this conservation is discoverable. As to the first assumption, it is only true in the case of divergent evolution. Many proteins are related by convergent evolution and accordingly, primary sequence comparison methods are insensitive to detecting such relationships. As to the second assumption, a number of factors can interfere with discovering conserved residues. First, incomplete, insubstantial, or only distantly related template sequence data can cause sequence comparison methods to break down. When there is incomplete or insufficient template sequence data, other methods, such as structure comparison methods, are required.

Since structural similarity is often conserved even at very low sequence homologies, or in the case of convergent evolution, structure comparison methods may be used for functional site annotation when primary sequence methods fail. Structure comparison methods may be classified as fold comparison methods or two-dimensional protein surface comparison methods. Fold comparison methods, as exemplified in CATH, SCOP and Dali assignments, are useful for making gross functional annotations, but they are of limited value for characterizing functional residues on the surface of a protein. Protein surface comparison methods are more useful for functional cluster annotation but are inherently harder to implement than sequence or fold comparison methods, since they are generally more complicated and require accurate three dimensional structures.

A number of approaches have been advanced for comparing protein surfaces. Brickmann et al., introduced a method that creates curvature profiles of a protein surface for comparing protein topography. Via A, Ferre F., Brannetti B., Helmer-Citterich M., Protein Surface Similarities: A Survey of Methods to Describe and Compare Protein Surfaces, Cell. Mol. Life Sci. 57: 1977-1979 (2000). Functional motif based approaches represent a functional cluster as a set of residues and corresponding distance constraints. Mitchell, E. M., Artymiuk, P. J., Use of Techniques Derived from Graph Theory to Compare Secondary Structure Motifs in Proteins, 243 J. Mol. Biol. 327-344 (1994). Other surface comparison methods use geometric hashing. Rose, M., Lin, S. L., Wolfson, H., Nussinov, R., Molecular Shape Comparisons in Searches for Active Sites and Functional Similarity, 11 Protein Eng. 269-288 (1998).

Still other functional site identification methods do not rely upon comparisons with known functional sites. Edelsbrunner's Alpha Shape theory, identifies functional sites with concave surface voids. Liang, J., Edelsbrunner, H., Woodward, C., Anatomy of Protein Pockets and Cavities: Measurement of Binding Site Geometry And Implications For Ligand Design, 7 Protein Sci. 1884-1897 (1998). http://sunrise.cbs.umn.edu/cast/background.html. Another method that identifies functional sites with voids on the surface or buried within a protein is the PASS algorithm. Brady, G. P., and Stouten, P. F., Fast Prediction and Visualization of Protein Binding Pockets with PASS, J. Comput. Aided Mol. Design, 14:383-401 (2000).

Thornton et al. recently introduced a neural network approach for identifying catalytic residues based upon a training set that characterizes residues by residue conservation, solvent accessibility, secondary structures, depth, and whether or not the residues lie in a cleft. Gutteridge, A., Barlett, G. J., Thornton J. M., Using a Neural Network and Spatial Clustering to Predict the Location of Active Sites in Enyzmes, J. Mol. Bio. 330:719-734 (2003).

The present invention generally relates to improved methods for annotating functional residues on the surface of a query protein. One aspect of the present invention uses a binary classification model to identify functional clusters of residues based upon comparisons with known functional clusters and putative functional clusters. The claimed methods statistically compare a putative functional site on the surface of query protein to a plurality of validated functional sites and putative functional sites derived from known functional proteins. Unlike approaches, such as Thornton's neural network approach which identifies individual catalytic residues based upon a residue-by-residue comparison scheme, the present methods use cluster based comparisons. More particularly, a putative functional site on the surface of a query protein is mapped into one of two half spaces corresponding to: 1) validated functional sites derived from a plurality of known functional proteins, and 2) putative functional sites on the surface of known functional proteins. Validated functional sites are known functional clusters of residues. Putative functional sites are either unknown functional sites—i.e. true functional residue clusters, or non-functional residue clusters. By comparing a putative functional cluster within this binary classification model the claimed methods are substantially more accurate than are other sequence or structure based comparison methods because the model can be trained to select away from false positives—i.e. annotating a site as functional when it is in fact non-functional. Further, even relative to Thornton's state-of-the-art neural networks based approach, the claimed methods are still far more accurate for the prospective reasons that geometric properties such as the depth of a residue is problematic to define absent the definition of a cluster. Lastly, cluster based methods offer the additional benefits of allowing a larger range of functional annotation scores to be used including, but not limited to the: 1) cluster “mouth area”; 2) cluster “mouth” circumference and 3) cluster volume.

A second aspect of the present invention uses functional annotation scores that reflect both sequence and structural conservation to represent putative functional sites (both on a query protein and on known function proteins) and validated functional sites within the comparison methods of the invention. A functional annotation score refers to a score that correlates an observable associated with a residue or a cluster or residues with biological function. By using functional annotation scores that are sensitive to both sequence similarity and structural similarity, the claimed comparison methods are sensitive to both convergent and divergent protein evolution.

A third aspect of the present invention is a method for determining a confidence score of a functional annotation based upon the distance between a putative functional cluster when mapped into the space used to represent validated functional sites and putative functional sites, and the plane that divides this space into two half spaces. By using a comparison scoring scheme that consider both sequence similarities and structural similarities, the claimed methods are more accurate at identifying functional sites than are the current schemes that only consider sequence or structural similarities.

### BRIEF DESCRIPTION OF THE FIGURES

FIG. 1a—Illustrates one method according to the invention for determining functional residues on the surface of a protein.

FIG. 1b—Illustrates one method according to the invention for determining a continuous SVM score.

FIG. 2a—Illustrates one method according to the invention for determining a plurality of residue conservation scores on the surface of a reference protein.

FIG. 2b—Illustrates an example of one method for scoring residue conservation.

FIG. 3—Illustrates an exemplary surface orientation score calculation.

FIG. 4—Illustrates one method according to the invention for determining a putative functional reference cluster.

FIG. 5a-c—Illustrate the application of the method illustrated in FIG. 4 for determining a putative functional reference cluster for the case of an exemplary protein surface comprising 28 residues.

FIG. 6—Illustrates another method according to the invention for determining a putative functional reference cluster.

FIGS. 7a-c—Illustrate the relationship between a putative functional reference cluster, its Voronoi diagram, its Delaunay tessellation and its Alpha Shape.

FIG. 8—Illustrates another method according to the invention for determining a putative functional reference cluster.

FIG. 9—Illustrates the geometric relationships between the volume, the surface area, the “mouth” area, and the depth of a putative functional reference cluster and its corresponding Delaunay tessellation/Alpha Shape.

FIG. 10—Illustrates the architecture of the executable files generated upon compiling the source code for Lin's SVM.

FIG. 11—Illustrates the relationship between training data and the optimal SVM hyperplane.

FIGS. 12a-d—Illustrate the multiple sequence alignment formed between one chain of PDB: 12asA and 28 template sequences.

FIG. 13—Illustrates the highest scoring binding site identified on PDB:12asA using the methods according to the invention.

FIG. 14—Compares the percentage of correct functional site identifications made using the methods according to the invention on a test set of 1188 proteins as a function of an SVM confidence score.

FIG. 15—Compares the relative accuracy of the methods according to the invention and the PASS algorithm on a test set of 82 proteins.

FIG. 16—Compares the identification of the binding site on Ferrochelatase using the methods according to the invention, and the top four identifications made by the PASS algorithm.

FIG. 17—Compares the identification of the novel statin binding site on Lymphocyte Function Associated Antigen-1 using the methods according to the invention, and the top three identifications made by the PASS algorithm.

FIG. 18—Compares the identification of the binding site on Glycogen Phosphorylase B using the methods according to the invention, and the top six identifications made by the PASS algorithm.

FIG. 19—Compares the identification of the binding site on Fructose 1-6-Biphosphatase using the methods according to the invention, and the top three identifications made by the PASS algorithm

FIG. 20—Compares the identification of the peptide exo-site on Factor VIIa using the methods according to invention, and the top three identifications made by the PASS algorithm.

FIG. 21—Compares the identification of the binding site on the P38 Kinase using the methods according to the invention and the top three identifications made by the PASS algorithm.

FIG. 22—Illustrates a system according to the invention.

### SUMMARY OF THE INVENTION

The present invention relates to improved methods for identifying functional residues on the surface of a query protein.

One aspect of the current invention compares a putative functional cluster on the surface of a query protein to a plurality of validated functional clusters and putative functional reference clusters derived from a plurality of reference proteins within a binary classification model in order to determine whether the putative functional cluster is a functional cluster.

A reference protein refers to any protein comprising a validated functional cluster on its surface. A validated functional cluster refers to a cluster of residues in a bound protein-ligand structure whose solvent accessible surface area increases upon removal of the ligand. Such clusters may be identified from the three dimensional structures of co-crystallized protein-ligand complexes. A convenient source for co-crystal structure data is the Protein Data Bank (“PDB”) which currently comprises over 1,000 co-crystals from a wide variety of protein families. Reference residues refer to those residues on the surface of a reference protein.

A putative functional cluster refers to a cluster of residues on the surface of a query protein that based upon one or more functional annotation scores or observables is identified as a potential functional cluster. An observable refers to a determinable quantity associated with a protein.

A putative functional reference cluster is a cluster of residues on the surface of a reference protein that, based upon one or more functional annotation scores or observables is identified as a potential functional cluster.

Functional annotation scores are used by the claimed methods to characterize and represent putative functional reference clusters, validated functional clusters and putative functional clusters. A functional annotation score refers to any score that generally reflects the likelihood that a particular residue or group of residues is functional. A functional annotation score may be one-dimensional, reflecting one observable, or multi-dimensional, reflecting multiple observables. Since functional clusters on the surface of a protein are generally characterized by evolutionarily significant residues and concave surface features such as depressions, clefts, grooves, and pockets, it is generally preferable to represent putative functional reference clusters, putative functional clusters and validated functional clusters with functional annotation scores that reflect both sequence conservation and structure conservation. One embodiment of the invention represents putative functional reference clusters, putative functional clusters and validated functional clusters with a four dimensional functional annotation score formed from the: 1) maximum neighbor averaged residue conservation z-score; 2) cluster depth score; 3) cluster surface area score, and 4) cluster “mouth” area score. The following sections will detail methods for determining the: 1)average residue conservation z-score for a cluster, 2) maximum residue conservation z-score for a cluster, 3) cluster surface area, 4) cluster volume, 5) cluster depth, 6) cluster mouth area, and 7) cluster mouth circumference as well as other functional annotation scores that are related to the foregoing.

The inventors observed that the statistical distribution of functional annotation scores, particularly the multi-dimensional distribution formed from the maximum neighbor average residue conservation z-score, cluster volume score, cluster depth score, and cluster mouth area score, that characterizes a plurality of putative functional reference clusters from a plurality of reference proteins, overlaps the same multi-dimensional distribution derived from a plurality of validated functional clusters. Since a putative functional reference cluster represents either a true functional cluster or a non-functional cluster, this observation indicates that the statistical distribution of functional annotation scores that characterize non-functional clusters, overlaps the distribution of functional annotation scores that characterize true functional clusters. If a putative functional cluster is to be compared with a plurality of putative functional reference clusters and validated functional clusters in order to determine whether the putative functional cluster is indeed functional, it is necessary to determine whether the putative functional cluster is more similar to the validated functional clusters, or more similar to the putative functional reference clusters. Determining the classification of a new object based upon the binary classification of a plurality of other objects is the well known binary classification problem in machine learning. Accordingly, the claimed methods use the methods for solving the binary classification problem to determine whether a putative functional cluster is functional, and therefore more similar to validated functional clusters, or non-functional, and therefore more similar to putative functional reference clusters. One embodiment according to the invention uses a support vector machine (“SVM”) for determining whether or not a putative functional cluster is indeed a functional cluster. A support vector machine represents the putative functional clusters, putative functional reference clusters and validated functional clusters in a vector space. The putative functional reference clusters and validated functional clusters form the “training set” used to generate the functional annotation model. The functional annotation model generated by the support vector machine consists of a hyperplane that divides the vector space used to represent the training set into two half spaces; one half space corresponding to the putative functional reference clusters and the other half space corresponding to the validated functional clusters. A putative functional cluster is assigned to one of the two half spaces based upon its representation in the space used to represent the training data. If a putative functional cluster falls into the half space that represents the putative functional reference clusters, it is annotated as a non-functional cluster. If a putative functional cluster falls into the half space that represents the validated functional clusters, it is annotated as a functional cluster. Accordingly, one method according to the invention for identifying functional residues on the surface of a query protein comprises the steps of: 1) determining at least one putative functional reference cluster on the surface of at least one reference protein; 2) determining at least one validated functional cluster on the surface of at least one reference protein; 3) determining a functional annotation score for each putative functional reference cluster determined in step 1) and each validated functional cluster determined in step 2); 4) determining a first set of functional annotation scores that characterizes the putative functional reference clusters determined in step 1) and a second set of functional annotation scores that characterizes the validated functional clusters determined in step 2); 5) determining at least one putative functional cluster on the surface of a query protein; 6) determining a functional annotation score for each putative functional cluster determined in step 5); and 7) determining whether each putative functional cluster is a functional cluster by comparing its corresponding functional annotation score to the first set of functional annotation scores that characterize the putative functional reference cluster and the second set of functional annotation scores that characterize the validated functional clusters.

Another aspect of the invention is a method for determining an SVM based functional annotation score based upon the distance between a functional annotation score used to represent a putative functional cluster and the optimal SVM hyperplane that divides the training data into two half spaces. Accordingly, one method according to the invention for determining an SVM based functional annotation score for a putative functional cluster comprises the steps of: 1) determining at least one putative functional reference cluster on the surface of at least one reference protein; 2) determining at least one validated functional cluster on the surface of at least one reference protein; 3) determining a functional annotation score for each putative functional reference cluster determined in step 1) and each validated functional cluster determined in step 2); 4) determining a first set of functional annotation scores that characterizes the putative functional reference clusters determined in step 1) and a second set of functional annotation scores that characterizes the validated functional clusters determined in step 2); 5) determining the optimal SVM hyperplane that separates the first set of functional annotation scores that characterizes the putative functional reference clusters and the second set of functional annotation scores that characterizes the validated functional clusters; 6) determining a functional annotation score that characterizes the putative functional cluster of the same form as the functional annotation scores determined in step 4); and 7) identifying the corresponding SVM based functional annotation score with the distance between the functional annotation score that characterizes the putative functional cluster and the optimal SVM hyperplane determined in step 5).

Further aspects of the invention are the methods for determining putative functional clusters and putative functional reference clusters for use in a binary classification model for functional annotation or for use in determining SVM based functional annotation scores.

Another aspect of the invention is a method for determining the probability that a putative functional reference cluster, characterized by a functional annotation score, is in fact functional. This aspect of the invention in based upon the realization that the co-crystallographic record deposited in the PDB provides a standard for the backtesting the accuracy of a functional annotation score including SVM based functional annotation scores.

Relevant Terminology.

Reference Protein—As used herein, it refers to a protein comprising a validated functional cluster.

Reference Structure—As used herein, it refers to the three-dimensional structure of the corresponding reference protein.

Reference Residue—As used herein, it refers to a residue on the surface of a reference protein

Reference Sequence—As used herein, it refers to the primary sequence of a corresponding reference protein.

Query Protein—As used herein, it refers to a particular protein for which the identification and characterization of any functional surface residues are sought using the methods according to the invention.

Query Structure—As used herein, it refers to the three-dimensional structure of the corresponding query protein.

Query Sequence—As used herein, it refers to the primary sequence of the corresponding query protein.

Query Residue—As used herein, it refers to a residue on the surface of query protein.

Validated Functional Cluster—As used herein, it refers to the cluster of residues in a bound protein-ligand structure whose solvent accessible surface area increases upon removal of the ligand.

Putative Functional Cluster—As used herein, it refers to a cluster of residues on the surface of a query protein that is identified as a potential functional cluster.

Putative Functional Reference Cluster—As used herein, it refers to a putative functional cluster on the surface of a reference protein.

Template Sequence—As used herein, it refers to a sequence which is homologous to either a reference sequence or another template sequence.

Concave Surface Feature or Surface Void—As used herein, it refers to a feature on the surface of a protein which may be characterized by a finite radius of curvature. Exemplary concave surface features include: clefts, pockets, grooves and surface depressions.

Residue Conservation Score—As used herein, it refers to a score which reflects the conservation of a residue on the surface of a protein relative to a plurality of template sequences.

Topography Score—As used herein, it refers to a score which reflects the geometric characteristics of a concave surface feature.

Functional Annotation Score—As used herein, it refers to any score that correlates an observable to protein function.

Reference Functional Cluster—As used herein, it refers to a validated functional cluster that has been “re-identified” using a functional annotation method for the purposes of backtesting the accuracy of the functional annotation method.

Continuous SVM Score—As used herein, it refers to a type of SVM determined functional annotation score.

Training Data—As used herein, it refers to the data within in a binary classification model that is used to train the classifier.

Testing Data—As used herein, it refers to the data-of-interest that is to be classified into one of two classes within a binary classification model.

### DETAILED DESCRIPTION OF THE INVENTION

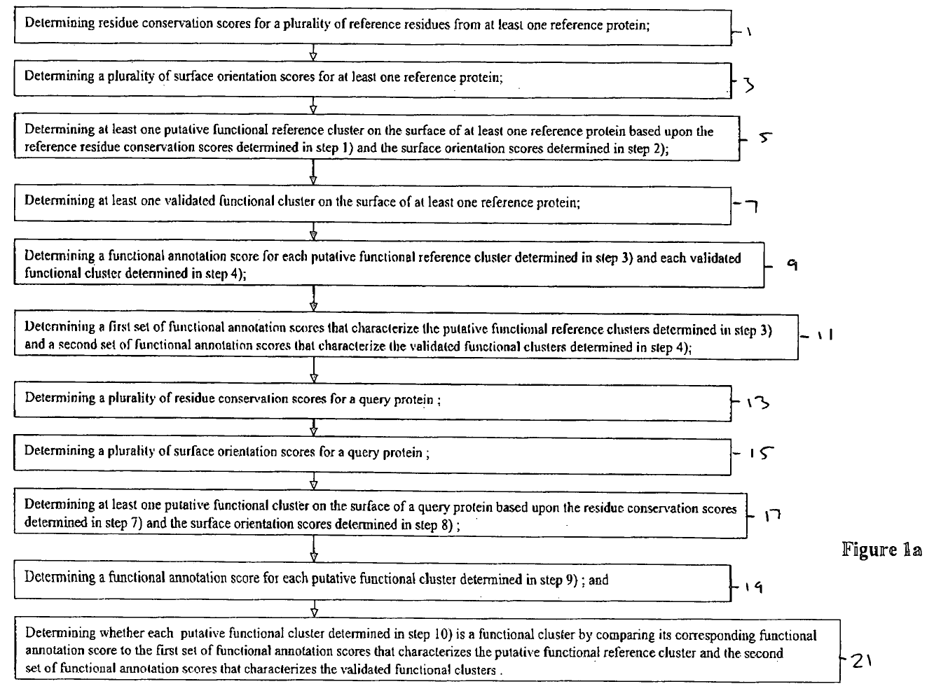

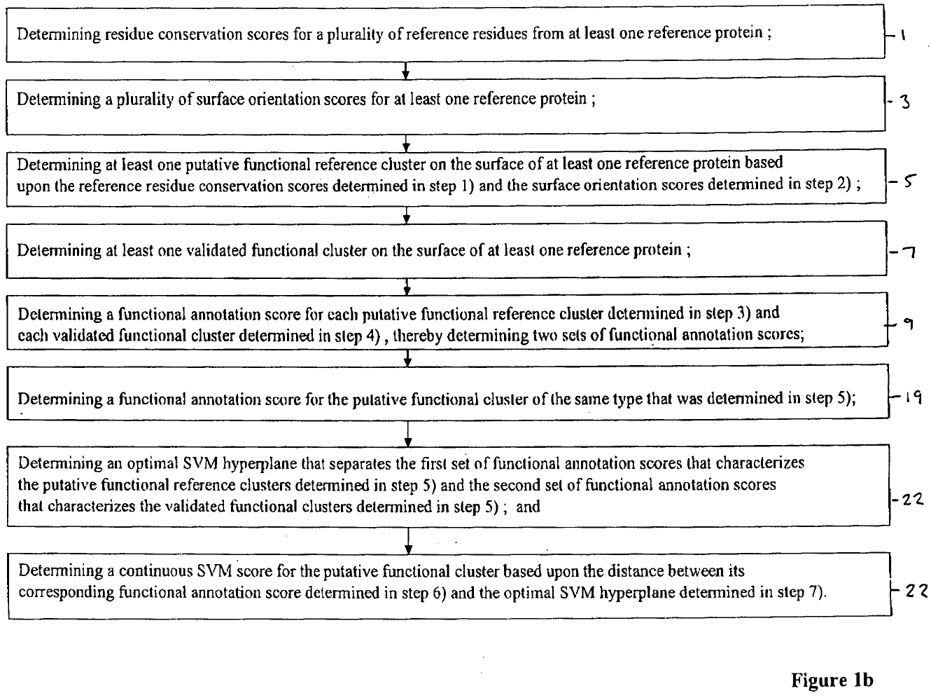

One method according to the invention for identifying functional residues on the surface of a query protein, that uses a preferred method for determining putative functional reference clusters and putative functional clusters, comprises the steps of: 1) determining residue conservation scores for a plurality of reference residues from at least one reference protein 1; 2) determining a plurality of surface orientation scores for at least one reference protein 3; 3) determining at least one putative functional reference cluster on the surface of at least one reference protein based upon the reference residue conservation scores determined in step 1) and the surface orientation scores determined in step 2) 5; 4) determining at least one validated functional cluster on the surface of at least one reference protein 7; 5) determining a functional annotation score for each putative functional reference cluster determined in step 3) and each validated functional cluster determined in step 4) 9; 6) determining a first set of functional annotation scores that characterize the putative functional reference clusters determined in step 3) and a second set of functional annotation scores that characterize the validated functional clusters determined in step 4) 11; 7) determining a plurality of residue conservation scores for a query protein 13; 8) determining a plurality of surface orientation scores for a query protein 15; 9) determining at least one putative functional cluster on the surface of a query protein based upon the residue conservation scores determined in step 7) and the surface orientation scores determined in step 8) 17; 10) determining a functional annotation score for each putative functional cluster determined in step 9) 19; and 11) determining whether each putative functional cluster determined in step 9) is a functional cluster by comparing its corresponding functional annotation score to the first set of functional annotation scores that characterizes the putative functional reference clusters and the second set of functional annotation scores that characterizes the validated functional clusters 21 determined in step 6). The method illustrated in FIG. 1a is based upon one method for determining putative functional clusters and putative functional reference clusters. However, the method illustrated in FIG. 1a may be generalized to any scheme for determining putative functional reference clusters and putative functional clusters. Methods for determining putative functional reference clusters and putative functional clusters will be detailed in the upcoming sections.

FIG. 1b illustrates one method according to the invention illustrated for determining a continuous SVM score for a putative functional cluster comprising the steps of: 1) determining residue conservation scores for a plurality of reference residues from at least one reference protein 1; 2) determining a plurality of surface orientation scores for at least one reference protein 3; 3) determining at least one putative functional reference cluster on the surface of at least one reference protein based upon the reference residue conservation scores determined in step 1) and the surface orientation scores determined in step 2) 5; 4) determining at least one validated functional cluster on the surface of at least one reference protein 7; 5) determining a functional annotation score for each putative functional reference cluster determined in step 3) and each validated functional cluster determined in step 4) 9, thereby determining two sets of functional annotation scores; 6) determining a functional annotation score for the putative functional cluster of the same type that was determined in step 5) 19; 7) determining an optimal SVM hyperplane that separates the first set of functional annotation scores that characterizes the putative functional reference clusters determined in step 5) and the second set of functional annotation scores that characterizes the validated functional clusters determined in step 5) 22; and 8) determining a continuous SVM score for the putative functional cluster based upon the distance between its corresponding functional annotation score determined in step 6) and the optimal SVM hyperplane determined in step 7) 22. The method illustrated in FIG. 1b is based upon one method for determining putative functional clusters and putative functional reference clusters. However, the method illustrated in FIG. 1b may be generalized to any scheme for determining putative functional reference clusters and putative functional clusters. Methods for determining putative functional reference clusters and putative functional clusters will be detailed in the upcoming sections. The section entitled Method for Determining the Probability that a Putative Functional Cluster is a Functional Cluster using Continuous SVM Scores or Other Functional Annotation Scores will detail how a functional annotation score, such as a continuous SVM score, may be use in combination with any method according to the invention for determining a putative functional cluster to determine the probability that a putative functional cluster is indeed a functional cluster.

Determining Residue Conservation Scores for a Plurality of Reference Residues from at Least One Reference Protein-1

A reference residue conservation score refers to a score that reflects the relative conservation of a residue on the surface of a reference protein relative to one or more template sequences. Reference residue conservation scores are first determined for a plurality of reference residues from at least one reference protein in order to identify putative functional reference clusters on the surface of the reference protein.

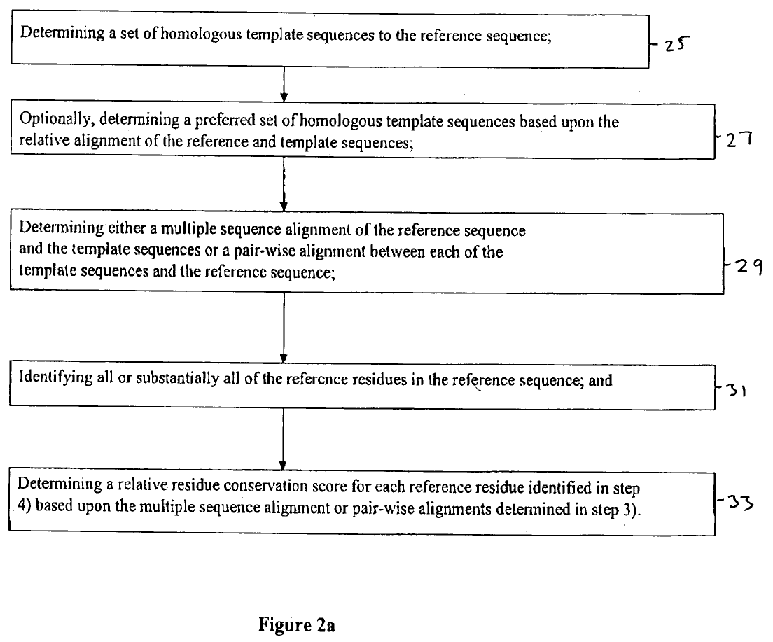

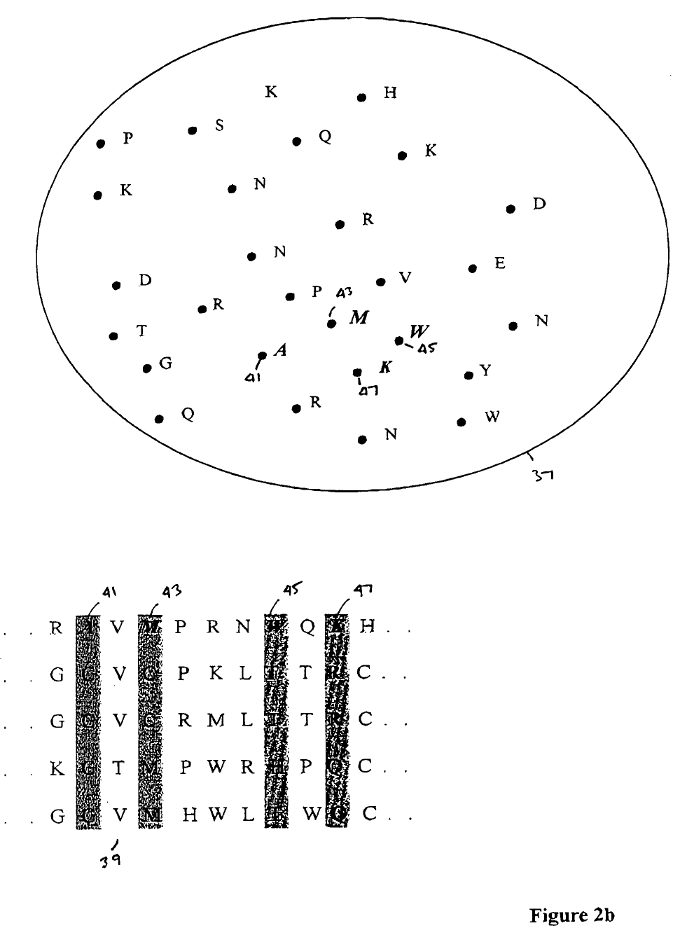

One method, illustrated in FIG. 2, for determining residue conservation scores for a plurality of reference residues comprises the steps of: 1) determining a set of homologous template sequences to the reference sequence 25; 2) optionally, determining a preferred set of homologous template sequences based upon the relative alignment of the reference and template sequences 27; 3) determining either a multiple sequence alignment of the reference sequence and the template sequences or a pair-wise alignment between each of the template sequences, and the reference sequence 29; 4) identifying all or substantially all of the reference residues in the reference sequence 31; and 5) determining a relative residue conservation score for each reference residue identified in step 4) based upon the multiple sequence alignment or pair-wise alignment determined in step 3) 33.

A template sequence refers to a sequence homologous to the query reference sequence that is used to determine residue conservation scores. A set of homologous template sequences may be determined 25 by running a sequence homology tool such as the various BLAST, Smith-Waterman, FASTA, Hidden Markov Model algorithms on the reference sequence using any large sequence database such as the NCBI Protein Sequence Database, http://www.ncbi.nlm.nih.gov.

Once a set of homologous template sequences has been determined, a second optional step 27, selects a preferred subset of these sequences for use in the multiple sequence alignment. This step is motivated by the realization that the sensitivity and specificity of sequence based comparison methods for functional annotation purposes may be increased by selecting those template sequences which are also of similar length and structure to the reference sequence and its corresponding structure. A preferred subset of homologous template sequences may be determined by selecting those template sequences which include alignment domains that do not vary by more than 20% in length from the corresponding alignment domain in the reference sequence. This simple length cut-off may be used alone or in combination with a threshold function, such as the HSSP function, which is sensitive to the percentage of continuously aligned residues, to determine a set of preferred template sequences. Sander, C; Schneider, R; Database of Homology Derived Protein Structures and the Structural Meaning of Sequence Alignment, Proteins, PROTEINS: Structure, Function, and Genetics, 9:56-58 (1991). The HSSP threshold function may be represented by:
Threshold=v+{100 (for L≦11), 480L^{0.32(1+exp(-L/1000)}(for 11<L≦450), 19.5 (for L>450)},
where v is an offset, and L is the length of the alignment between two sequences. The HSSP threshold function provides a lower threshold of sequence similarity, as a function of alignment length, for those alignments which are likely to produce a proper homology model. Alternatively, one skilled in the art could derive a comparable expression based upon sequences and structures in a databank containing a broad cross section of sequences and corresponding structures, such as the PDB.

Another sorting method sorts a set of template sequences based upon their phylogenetic relationship using phylogenetic tree based scoring schemes known to one ordinarily skilled in the art. A phylogenetic tree represents each sequence as a “leaf”; related sequences form “branches”. The evolutionary relationship, and therefore the degree of sequence conservation, may be represented by the distance between leaves and branches. A cut-off distance between branches or leaves may be selected to determine a preferred set of template sequences. Such a distance may be determined by one ordinarily skilled in the art by back-testing predicted structures based upon sequences and structures in a databank containing a broad cross-section of sequences and corresponding structures, such as the PDB.

Once a set of preferred template sequences has been determined, a third step 29 determines a multiple sequence alignment of the reference sequence and its homologous template sequences. A multiple sequence alignment may be determined using any multiple sequence alignment tool known in the art, such as Clustal W. J. D. Thompson, D. G. Higgins, T. J. Gibson, Nucl. Acids Res. 22, 4673-4680 (1994). Alternatively, a multiple sequence alignment can be avoided by computing pair-wise alignments between each of the template sequences and the reference sequence.

The fourth step 31, identifies all or substantially all of the reference residues.

The fifth step 33, determines the conservation of the reference residues identified in step four relative to the multi-sequence alignment. The conservation of a particular reference residue is represented by its raw residue conservation score. Normalized residue conservation scores may be determined by normalizing the raw residue conservation scores. Raw residue conservation scores may be based upon any method which represents the residue conservation across the multi-sequence alignment including Shannon entropy calculations, pair-wise mutation calculations, or evolutionary trace methods. Normalized conservation scores, may be determined from the p-value, z-value or any other scheme that represents the statistical significance of a particular raw residue conservation score.

Both raw residue conservation scores and normalized residue conservation scores may be averaged over neighbor residues to “smooth” out residue conservation scoring over the surface of a protein. One method averages the residue conservation score of a first residue with the scores of those residues that are “touching” the first residue. A second residue is said to be touching a first residue if the distance between the center of any heavy atom, m, in the first residue and the center of any heavy atom, n, in the second residue is less than or equal to r_{1,m}+r_{2,n}+2rₛₒₗᵥₑₙₜ, where r_{1,m} represents the radius of a heavy atom in the first residue, r_{2,n} represents the radius of a heavy atom in the second residue and rₛₒₗᵥₑₙₜ represents the radius of a solvent molecule. Another neighbor averaging scheme averages over both those residues that are touching a first residue—the first order touching residues, and those residues that are touching the first order touching residues—the second order touching residues. The following section will detail how residue conservation scores may be used to identify putative functional clusters.

Since the accuracy of the claimed methods increases both as the number of putative functional reference clusters increases and as the structural diversity of the putative functional reference clusters increases, it is generally preferable to determine as many reference residue conservation scores from as many different reference structures as is computationally practicable. Further, it is generally preferable to determine as many residue conservation scores as is computationally practicable since it increases the residue conservation scoring density across the surface of a reference structure and accordingly, increases the accuracy of putative functional cluster identifications.

The following example illustrates how a raw residue conservation score is determined using the methods illustrated in FIG. 2a and using a Shannon entropy residue conservation scoring function. FIG. 2b illustrates an exemplary reference protein 37 and a fragment of its corresponding sequence 39 that comprises 4 reference residues 41, 43, 45, 47. The Shannon entropy residue conservation scoring function for a reference residue, i, is given by

$${r}_{i} = {\sum }_{j}^{} {p}_{j}$$

n pⱼ where pⱼ is the observed probability of finding a particular residue type j in the same column as i. For reference residue 41, there are only two types of residues in its column: G and A. The probability of observing G is ⅘ and the probability of observing A is ⅕. Accordingly, r₁=0.2 ln 0.2+0.8 ln0.8. It follows that for the second reference residue 43, r₂=0.4 ln0.4+0.6 ln0.6, for the third reference residue 45, r₃=0.2 ln 0.2+0.2 ln0.2+0.6 ln0.6 and for the four reference residue 47, r₄=0.2 ln 0.2+0.4 ln0.4+0.4 ln0.4. The residue conservation z-score of a particular raw residue conservation score, z(rᵢ), may be determined from the distribution of raw residue conservation scores determined from the reference sequence as a whole.

Determining a Plurality of Surface Orientation Scores for at Least One Reference Protein-3.

A surface orientation score represents the local curvature at a point on the surface of a protein—i.e. whether it is convex or concave. The claimed methods determine a plurality of surface orientation scores across the surface of at least one reference protein to determine its curvature. The surface orientation scores are then used in combination with the residue conservation scores from the same reference protein to identify a putative functional reference cluster on that reference protein. There is no inherent limitation on how surface orientation scores may be determined. One method for determining a surface orientation score for a reference residue i, referred to herein as the vector dot-product method, determines the dot-product of a vector defined normal to i with each vector that connects i to its nearest neighbors. If, for example, a particular query residue has 5 nearest neighbors, this method would generate 5 dot-product values ranging from 1 to −1 depending upon the local geometry of that query residue and its nearest neighbors. In a next step, the local curvature may be determined by summing those dot-product values that are greater than zero and dividing the sum by the number of dot-product values. Accordingly, a surface orientation score of zero would correspond to a locally convex surface and a surface orientation score of 1 would correspond to a locally concave surface. An intermediate score would indicate that the local surface is corrugated. This scheme may be applied to a plurality of reference residues to map the local curvature of a reference structure.

Since the accuracy of the claimed methods increases both as the number of putative functional reference clusters increases and the structural diversity of the putative functional reference clusters increases, it is generally preferable to determine as many surface orientation scores from as many different reference structures as is computationally practicable. Further, it is generally preferable to determine as many surface orientation scores for a given reference protein as is computationally practicable since it increases the surface orientation score density across the surface of a reference structure and accordingly, increases the accuracy of putative functional reference cluster identifications.

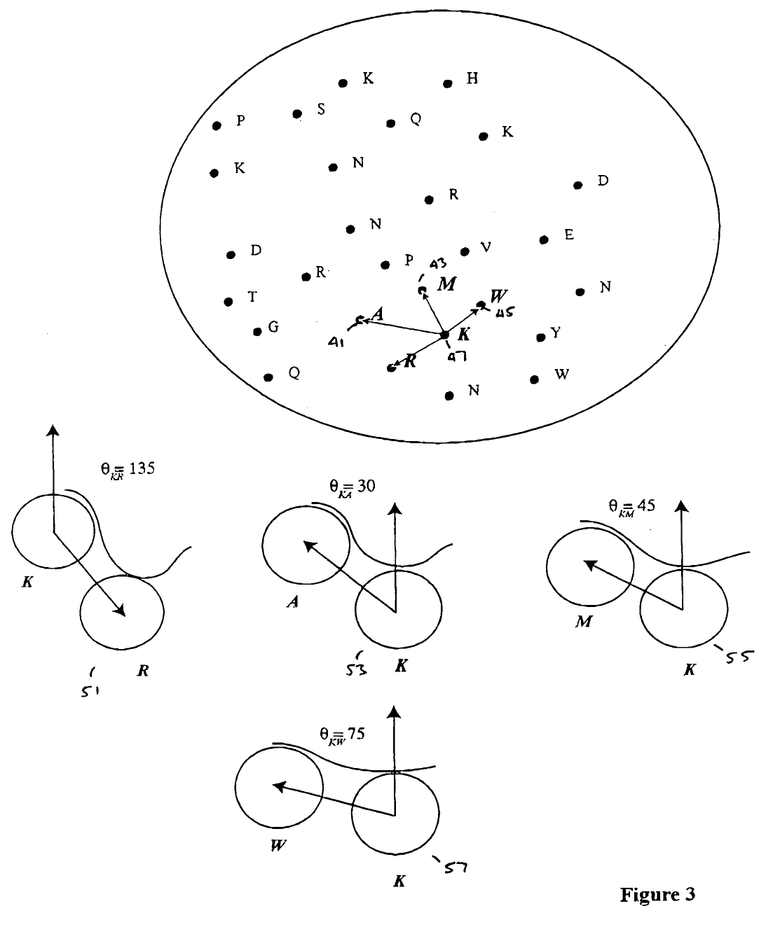

FIG. 3 illustrates an exemplary calculation of a surface orientation score using the protein surface illustrated in FIG. 2b. Assume that residue 47 is “touched” by four residues, 41, 43, 45 and 49. Further assume the relative geometry of 41, 43, 45 and 49 is illustrated in the radial cross-sections 51, 53, 57 and 59 also illustrated in FIG. 3. A first step in the surface orientation score calculation determines a unit vector from 47 normal to the surface of the protein 37. A second step determines unit vectors, \hat{R}, Â, \hat{M} and Ŵ between 47 and 41, 43, 45 and 49. A next step determines the dot-products: \hat{K}·Â, \hat{K}·\hat{M}, \hat{K}·Ŵ and \hat{K}·\hat{R}. Only 3 dot-product values are greater than or equal to zero: \hat{K}·Â, \hat{K}·\hat{M}, and \hat{K}·Ŵ. Accordingly, the surface orientation score for residue 47 is determined by
S.O._{K}\hat{K}·Â+\hat{K}·\hat{M}+\hat{K}·W/4=0.25(cos 30°+cos 45°+cos 75°)=0.458.

Methods for Determining at Least One Putative Functional Reference Cluster on the Surface of at Least One Reference Protein-5.

A putative functional reference cluster is a cluster of residues on the surface of a reference protein that, based upon one or more observables is identified as a potential functional cluster. Since functional sites typically contain from ten to approximately thousand residues, putative functional reference clusters should contain at least five residues and less than 1000 residues. Putative functional reference clusters represent two possibilities: 1) true functional clusters on the surface of a reference structure—e.g. non-validated functional clusters; or 2) non-functional clusters. In order to minimize the likelihood of annotating a putative functional cluster as functional when it is in fact non functional (i.e making a false positive functional annotation), the claimed methods use putative functional reference clusters, or more particularly the functional annotation scores that characterize putative functional reference clusters, as one of the two classes of training data within a binary classification model. In this model, putative functional reference clusters are identified as “false” functional clusters. The other class of training data, validated functional clusters, are considered as functional clusters, or equivalently, “true” functional clusters within this model.

Any of the methods known in the art for identifying functional clusters such as the PASS algorithm, CAST-P algorithm or any of the methods detailed in the Introduction may be used to identify putative functional reference clusters. Since functional clusters are often times identified with conserved residues and concave surface features, functional annotation scores associated with either of these aspects of functional clusters may be used to identify putative functional reference clusters. One method for identifying a putative functional reference cluster comprises the steps of: 1) determining residue conservation scores for a plurality of reference residues; 2) identifying a cluster of connected query residues; 3) determining the average residue conservation score of the residues that comprise said cluster; 4) determining the average residue conservation score of those residue that do not comprise said cluster; and 5) if the average determined in step 3) is greater than the average determined in step 4), selecting said cluster as a putative functional reference cluster. Another method for identifying a putative functional reference cluster comprises the steps of: 1) identifying a void on the surface of a reference protein; 2) determining the volume of said void; 3) comparing the volume of said void to the volume of a water molecule; and 4) if the volume of said void is greater than the volume of a water molecule, selecting said cluster as a putative functional reference cluster.

The approaches in the following subsection offer the prospective advantage of using functional annotation scores relating to sequence conservation and structural information in order to identify putative functional reference clusters.

The condition of determining putative functional reference clusters from at least one reference structure is intended to reflect that there is no general limitation on the number of reference structures that must be analyzed. Since putative functional reference clusters are identified in order to determine the functional annotation scores that characterize putative functional reference clusters, for the same reasons as discussed in the section immediately above, it is preferable, although not necessary, to determine putative functional reference clusters from as many reference structures as is computationally practicable.

Residue Conservation Score and Surface Orientation Score Based Approaches for Determining a Putative Functional Reference Cluster.

In one method according to the invention, a putative functional reference cluster is identified based upon whether a cluster of solvent accessible reference residues are characterized by residue conservation scores and surface orientation scores that diverge from residue conservation scores and surface orientation scores across the surface of the reference protein. This identification scheme takes advantage of the fact that many functional clusters may be characterized by strongly conserved, solvent accessible residues organized as pockets, clefts, grooves, depressions or other concave surface features.

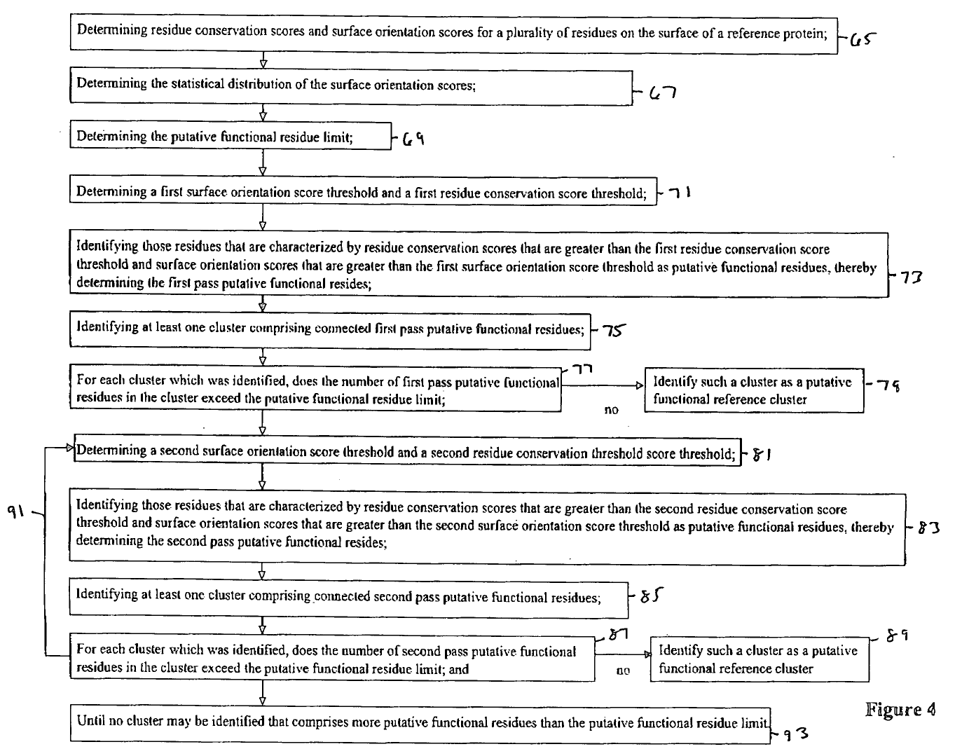

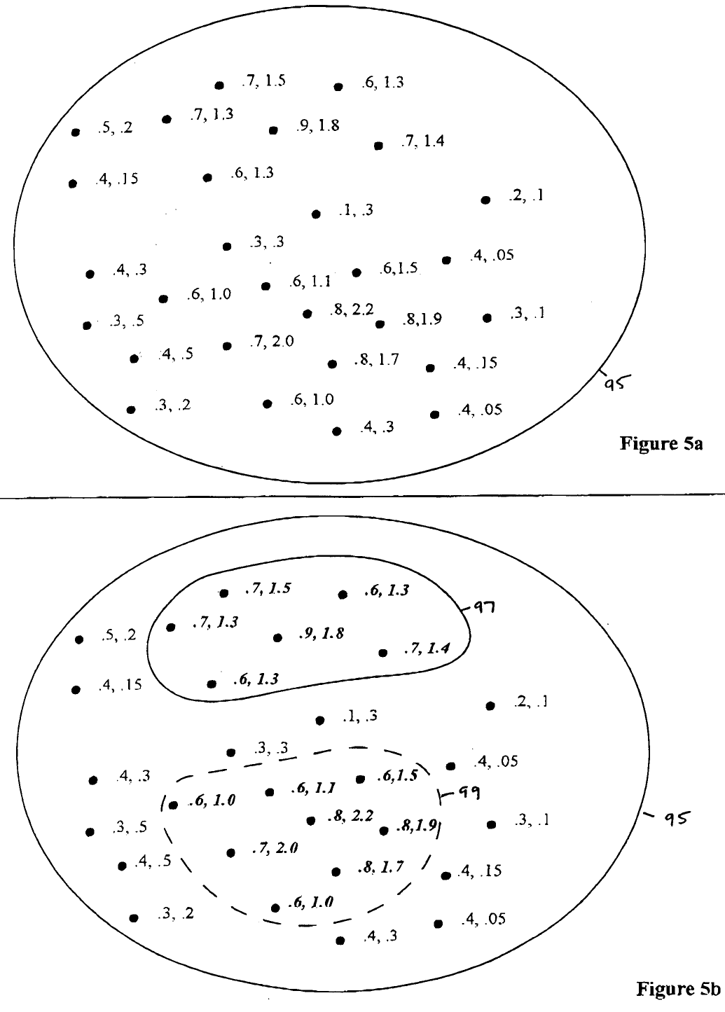

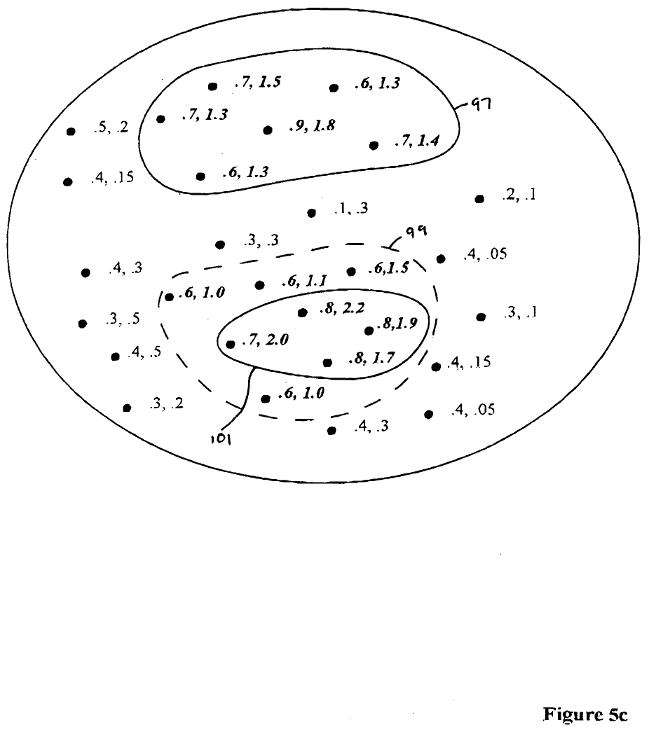

FIG. 4 illustrates one method according to the invention for determining a putative functional reference cluster from a plurality of residue conservation and surface orientation scores. A first step 65, determines residue conservation scores and surface orientation scores for a plurality of residues on the surface of a reference protein.

A second step 67, determines the statistical distribution of the surface orientation scores.

A third step determines the putative functional residue limit 69. One method for determining the putative functional residue limit identifies the limit with the number of surface orientation scores that comprise the largest peak in the surface orientation score distribution that may be identified with concave surface orientation scores. Since functional sites are often characterized by concave surface features it may be expected that the distribution of surface orientation scores should have a peak on the right side of the distribution—i.e. the concave side of the distribution. For the case where surface orientation scores range from 0 to 1, where 0 represents a convex score and 1 represents a concave score, the largest peak centered about a surface orientation score greater than 0.5 may be used. In one embodiment of the invention the surface orientation scores are divided into a plurality of statistical bins of finite width. For example, if a surface orientation score distribution from 0-1 is divided into 50 statistical bins, each bin would have a width of 0.02. Thus, the putative functional residue limit would be identified with the number of surface orientation scores in the statistical bin that has the greatest number of surface orientation scores greater than 0.5.

A fourth step determines a first surface orientation score threshold and a first residue conservation score threshold 71. Generally, these first thresholds should be chosen sufficiently broadly to minimize false negative annotations—i.e. minimize the probability of identifying putative functional residues as non-functional when they are in fact functional. For the case where surface orientation scores range from 0 to 1 and where residue conservation scores are expressed with z-scores, a first surface orientation score threshold of 0.4 and a first residue conservation score threshold of 0.5 may be selected. Accordingly, those residues with surface orientation scores greater than 0.4 and z-scores greater than 0.5 are identified as putative functional residues. A first surface orientation score threshold of 0.4 is selected because it assures that even flat, or corrugated features, with surface orientation scores of approximately 0.5 will be initially sampled. A first residue conservation score threshold of 0.5 is selected because it assures that even residues characterized with residue conservation z-scores that are within half a standard deviation of the average residue conservation z-score will be initially sampled.

A fifth step 73, identifies those residues that are characterized by residue conservation scores that are greater than the first residue conservation score threshold, and surface orientation scores that are greater than the first surface orientation score threshold, as putative functional residues. Such residues are referred to as first pass putative functional residues since they are defined by reference to the first surface orientation score threshold and the first residue conservation score threshold.

A sixth step 75, identifies at least one cluster of connected first pass putative functional residues. A first putative functional residue is said to be connected to a second putative functional residue if the first putative functional residue is touching the second putative functional residue. For each such cluster identified 77, if the number of connected first pass putative functional residues does not exceed the putative functional residue limit, such a cluster is denoted as a putative functional reference cluster 79.

If a cluster comprising more connected first pass putative functional residues than the putative functional residue limit is identified, a seventh step 81, selects a second surface orientation score threshold and a second residue conservation score threshold such that both second threshold scores tend more towards functional scores than the initial score thresholds—e.g. tend more towards concave surface features and conserved residues. For the case where surface orientation scores are measured from 0 to 1 and residue conservation scores use z-scores, a second surface orientation score threshold of 0.5 and a second residue conservation score threshold of 0.7 may be selected.

An eighth step 83, identifies those residues in each cluster (namely, those clusters that comprise more connected first pass putative functional residues than the putative functional residue limit) that are considered functional based upon the second set of threshold scores determined in step seven. Such functional residues are referred to as second pass putative functional residues.

A ninth step 85, identifies at least one cluster comprising connected second pass putative functional residues. For each such cluster identified 87, if the number of connected second pass putative functional residues does not exceed the putative functional residue limit, such a cluster is denoted as a putative functional reference cluster 89.

If a cluster comprising more connected second pass putative functional residues than the putative functional residue limit is identified, a tenth step 91, repeats the seventh step, thereby selecting a third surface orientation score threshold and a third residue conservation score threshold such that both third threshold scores tend more towards functional scores than the second set of score thresholds—i.e. tend still more towards concave features and conserved residues. Steps 7-10 are repeated a plurality of times, each time narrowing the allowed residue conservation and surface orientation score ranges, until no clusters may be identified that comprise more connected putative functional residues than the putative functional residue limit 93.

It will also be appreciated by one skilled in the art that a number of variations on this method may be employed. Instead of identifying the putative functional residue limit with the number of surface orientation scores under the largest peak associated with concave scores in the surface orientation distribution, the total number of surface orientation scores greater than 0.8 may be identified with the putative functional residue limit. Alternatively, the putative functional residue limit may be identified with the number of residue conservation scores under the largest peak centered about a residue conservation z-score greater than 1.0. A still further variation may identify the putative functional residue limit with the total number of residue conservation scores greater than 1.0 Another variation on the methods illustrated in FIG. 4 may use the putative functional residue limit as an exact limit rather than a lower limit—i.e. the total number of putative functional residues between all putative functional reference clusters is equivalent to the putative functional residue limit.

In order to illustrate the application of the methods illustrated in FIG. 4, consider the exemplary protein surface 95 illustrated in FIGS. 5a-c. The exemplary surface in FIG. 5a consists of 28 residues, each characterized by a surface orientation score that ranges from 0 (convex) to 1 (concave), and a residue conservation z-score. Further, assume that a putative functional residue limit of 7 was determined from the distribution of surface orientation scores. A first step selects an initial surface orientation score threshold of 0.4 and an initial surface residue conservation score threshold of 0.4. A next step compares the residue conservation scores and surface orientation scores to the first residue conservation score threshold and the first surface orientation score threshold, respectively, for each or substantially each of the residues on the exemplary surface in order to identify first pass putative functional residues. A next step illustrated in FIG. 5b, determines clusters of connected first pass putative functional residues. FIG. 5b illustrates two such clusters 97, 99. Since the upper cluster 97 comprises 6 connected first pass putative functional residues, it is identified as a putative functional reference cluster. Since the lower cluster 99 comprises 8 first pass putative functional residues, a next step determines a second surface orientation score threshold of 0.7 and a second residue conservation score threshold of 1.0. A next step determines second pass putative functional residues and identifies any clusters of connected second pass putative functional residues. FIG. 5c illustrates one such cluster 101 which is also identified as a putative functional reference cluster since it comprises less than 7 second pass putative functional residues. Since no clusters remain that comprise more putative functional residues than the putative functional residue limit, the search stops.

One of the benefits of this method for identifying putative functional reference clusters is that it requires no assumptions except that functional clusters are characterized by conserved solvent accessible residues which have a local curvature that varies from the curvature found elsewhere on the surface of the reference protein.

This iterative method for determining putative functional reference clusters also further illustrates why it is generally preferable, although not necessary, to determine residue conservation and surface orientation scores for all or substantially all of the surface residues of a reference protein. As the surface coverage for residue conservation scores and surface orientation scores increases, the geometry of putative functional reference clusters may be defined more accurately. Still, under certain circumstances, such as the identification of a very large functional cluster, the claimed methods may still sufficiently identify a putative functional reference cluster without surface orientation and residue conservation scores for each or substantially each reference residue. For example, once again assume that each residue on the surface of a reference structure is coordinated by four nearest neighbors. Further assume that a large active site typically contains about 100 residues. Since the methods according to the invention rely in-part on the observation that functional sites are often characterized by a cluster of evolutionarily significant, topologically distinct residues, even if surface orientation scores and residue conservation scores are calculated for every tenth residue on the surface of a reference protein, a large functional site of approximately 100 residues could still be identified from a cluster of 10 surface orientation and residue conservation scores.

Void Based Methods for Determining a Putative Functional Reference Cluster.

Many functional clusters are characterized by concave surface features such as grooves, pockets and clefts on the surface of a protein. Accordingly, when there is no, or insufficient template sequence data, putative functional reference clusters may still be identified with concave, voids on the surface of a reference protein. There is no inherent limitation on how clusters of concave, solvent accessible residues may be identified on the surface of a reference protein. Either numerical or analytical methods may be employed. Numerical methods, such as the various grid based approaches, represent the surface or the sub-surface of a protein within a framework of a three-dimensional lattice of cells. One grid method represents the surface of a protein with a plurality of points and corresponding normal vectors. Via, A., Ferre, F., Brannetti, B., Helmer-Citterich, M., Protein Surface Similarities: A Survey of Methods to Describe and Compare Protein Surfaces, Cell. Mol. Life Sci. 57: 1979-1987 (2000). The shell of points and vectors is then superimposed with a lattice of cubic cells. Each point is then represented by its corresponding cubic face. A putative functional reference cluster may be identified with a void on the surface of a reference protein where the void volume is greater than the volume of a solvent molecule. The void volume may be determined by summing the volume of the cubic cells that comprise the cluster.

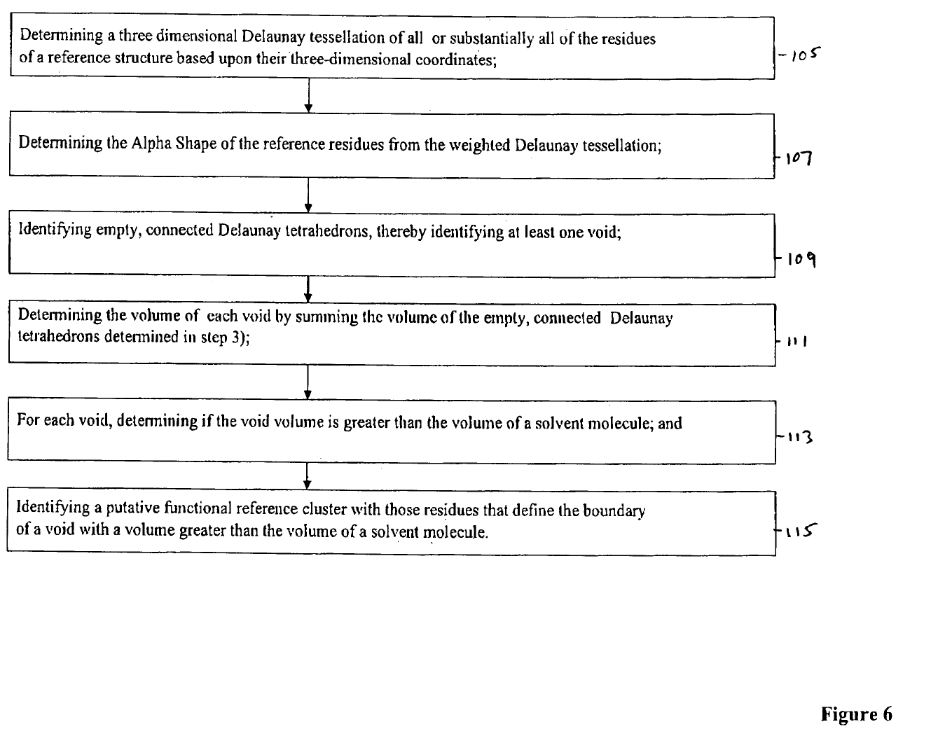

An analytical method, illustrated in FIG. 6, that may be used to identify a solvent accessible void on the surface of a reference protein and thereby identify a putative functional reference cluster comprises the steps of: 1) determining a three dimensional Delaunay tessellation of all or substantially all of the residues of a reference structure based upon their three-dimensional coordinates 105; 2) determining the Alpha Shape of the reference residues from the Delaunay tessellation 107; 3) identifying empty, connected Delaunay tetrahedrons, thereby identifying at least one surface void 109; 4) determining the volume of each void by summing the volumes of the empty, connected Delaunay tetrahedrons determined in step 3) 111; 5) determining if each void volume is greater than the volume of a solvent molecule 113; and 6) identifying a putative functional reference cluster with those residues that define the surface of a void with a volume greater than the volume of a solvent molecule 115.

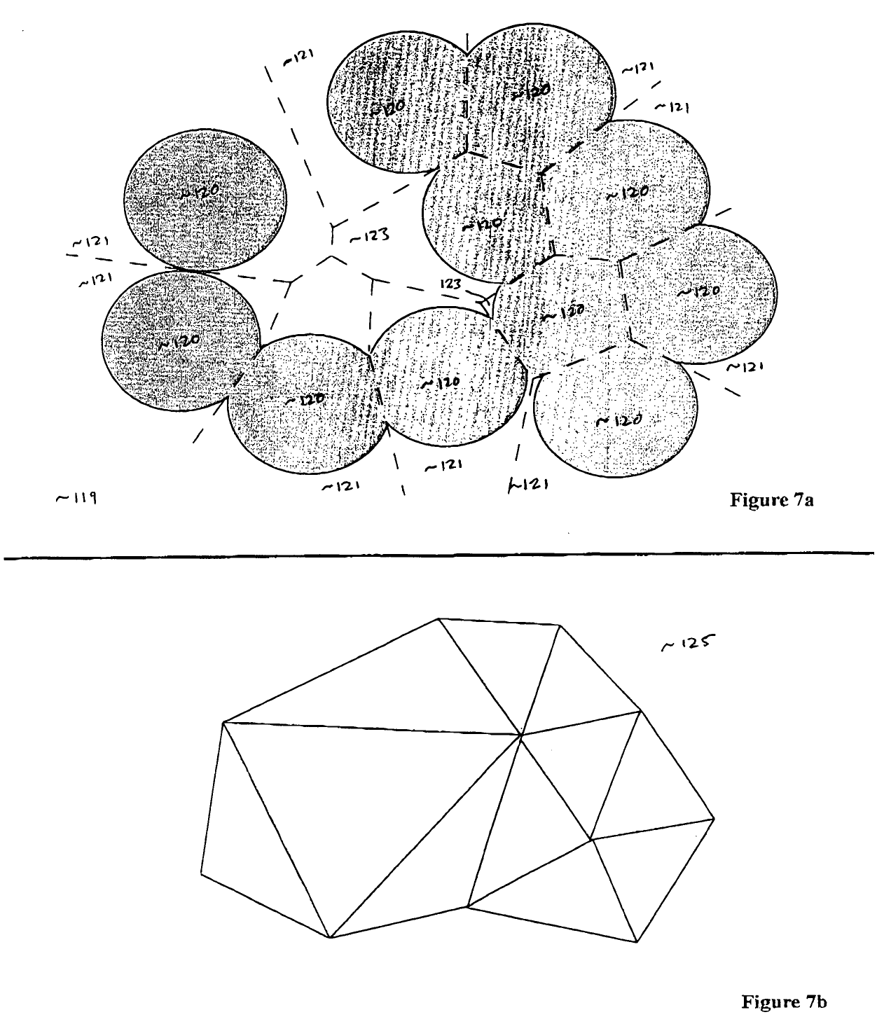

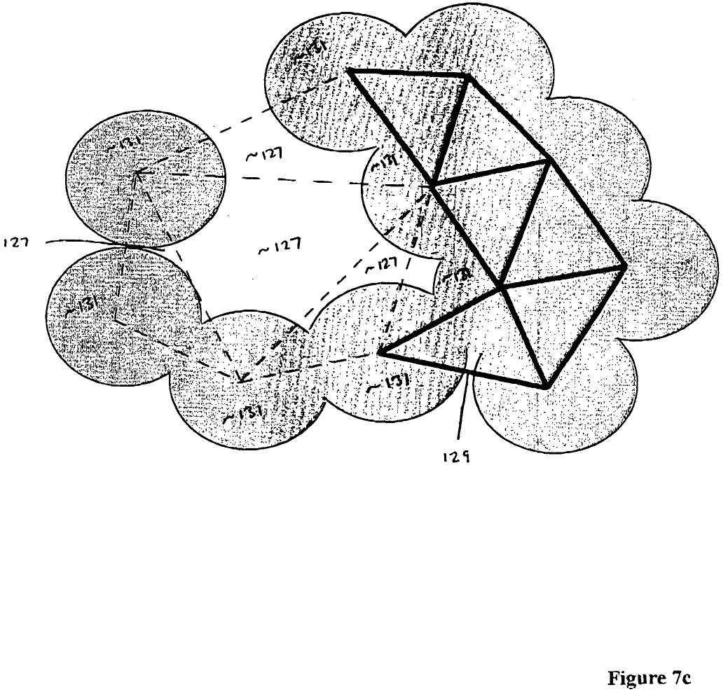

FIGS. 7a-c illustrate the relationship between a putative functional reference cluster, its Delaunay tessellation, its Alpha Shape and the determination of voids. The Delaunay tessellation is mathematically equivalent to, and may be derived from, the Voronoi diagram of a residue cluster. FIG. 7a illustrates a radial cross-section of an exemplary cluster 119 comprising 11 atoms and its corresponding Voronoi diagram. It will be appreciated by one skilled in the art that this cluster of 11 atoms is intended for illustrative purposes only. Actual residue clusters will comprise far more than 11 atoms. It will further be appreciated by one ordinarily skilled in the art that since this illustrative void is represented in two dimensions, its surface area is compared to the surface area of a solvent molecule. The van der Waals radii of a water molecule is 1.4 A (A=Angstroms). In a true implementation of this method, the volume of the void as defined by empty Delaunay tetrahedrons would be compared to the volume of a solvent molecule. The van der Waals volume of a water molecule is 11.5 A³. The Voronoi diagram comprises a plurality of Voronoi cells. Each Voronoi cell contains one atom 120. The area of each cell is defined such that the distance of each point within a particular Voronoi cell is closer to the atom of that cell than any other atom. Accordingly, two types of Voronoi cells may be identified in a Voronoi diagram: 1) open sided polygons for those boundary atoms that form the convex hull 121; and 2) closed polygons 123. FIG. 7b illustrates the Delaunay tessellation 125 corresponding to the Voronoi diagram 119 illustrated in FIG. 7a. It is formed by drawing a segment across every Voronoi edge that separates two Voronoi cells and connecting the respective atom centers of the two cells. FIG. 7c illustrates the Alpha Shape corresponding to the Delaunay tessellation illustrated in FIG. 7b. It is formed by subtracting those triangles 127 from the Delaunay tessellation that correspond to Voronoi edges or vertices outside of the functional cluster. Such triangles are referred to as “empty” Delaunay triangles 127. FIG. 7c shows these omitted edges and corresponding empty triangles with dashed lines and the corresponding alpha shape 129 with bold, solid lines. In this example, a void may be identified with the four empty Delaunay triangles 127.

The surface area of this two dimensional void, may be found by summing the areas of the empty Delaunay triangles less the surface area of those triangles within the atom disks. The atoms 131 are identified as forming the boundary of the void. If the surface area of the void defined by the empty Delaunay tetrahedrons exceeds the surface area of a solvent molecule, the atoms 131 are identified as a putative functional reference cluster (in two dimensions).

The Delaunay tessellation 105 of the reference residues may be calculated based upon their structural coordinates and their corresponding van der Waals radii. Tables of van der Waals radii are readily available. If the reference structure is also found in the Protein Data Bank, the atomic radii may be assigned using the utility program PDB2ALF which is available for download at http://www.alphashapes.org/alpha/. The weighted Delaunay tessellation 105 and Alpha Shape 107 computations may be performed using the programs DELCX and MKALF, respectively. Both are also available for download at http://www.alphashapes.org/alpha/.

Another method for determining the Delaunay tessellation of the reference residues calculates the Delaunay tessellation based upon a surface averaged shell representation of the reference structure. A method for determining a surface averaged shell representation of a reference structure comprises the steps of: 1) selecting solvent accessible residues on the surface of the reference protein; 2) determining solvent accessible side chains; 3) replacing solvent accessible side chains with beta Carbon atoms or pseudo atoms; and 4) forming the surface averaged shell representation from the solvent accessible residues and beta-Carbon/pseudo atom replacements to the side chains. By representing a reference structure with a surface averaged shell representation, significant computational efficiencies may be gained relative to a “complete” representation of a reference structure. At least two further advantages may be identified with this representation: 1) greater sensitivity and specificity to shallow surface features since surface irregularities are smoothed; and 2) greater sensitivity and specificity in general when using homology modeled reference structures since this method replaces the side chains which are often modeled incorrectly with homology modeling techniques with pseudo atoms.

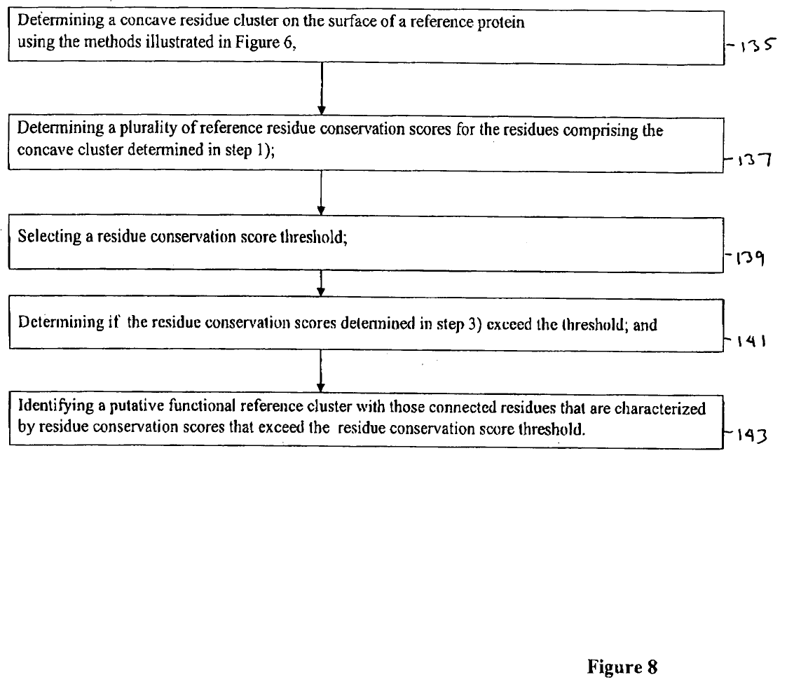

If residue conservation data is available, another method for determining putative functional reference clusters, illustrated in FIG. 8, comprises the steps of: 1) determining a concave solvent accessible residue cluster on the surface of a reference protein using the methods illustrated in FIG. 6, 135; 2) determining a plurality of reference residue conservation scores for the residues comprising the concave cluster determined in step 1) 137; 3) selecting a residue conservation score threshold 139; 4) determining if the residue conservation scores determined in step 3) exceed the threshold 141; and 5) identifying a putative functional reference cluster with those connected residues that are characterized by residue conservation scores that exceed the residue conservation score threshold 143.

A residue conservation score threshold may be fixed or variable. A residue conservation score threshold may be determined from the distribution of residue conservation scores from the reference protein as a whole. If residue conservation scores use z-scores, an exemplary scheme for determining a fixed residue conservation score threshold may select the center of the largest peak in the residue conservation score distribution centered about a residue conservation z-score greater than 1.0. Alternatively, the residue conservation score threshold may be variable and used in conjunction with a putative functional residue limit in an iterative scheme similar to the one detailed in the section titled, Surface Orientation Score Based Approaches for Determining a Putative Functional Reference Cluster.

Determining at Least One Validated Functional Cluster for at Least One Reference Protein-7.

The claimed methods use validated functional clusters, or more particularly, the functional annotation scores that represent validated functional clusters, as one of the two classes of training data within a binary classification model for determining whether a putative functional cluster is a functional cluster. Validated functional clusters are “true” functional clusters within this model. A validated functional cluster may be immediately identified from the three dimensional structure of a reference protein. Since validated functional clusters are identified in order to determine functional annotation scores that characterize a “true” functional cluster, for the same reasons as discussed in the section immediately above, it is preferable, although not necessary, to determine validated functional clusters from as many reference structures as is computationally practicable. Still, under certain circumstances, as has been detailed before, the claimed methods may sufficiently determine validated functional clusters from as few as one reference structure. For example, where the methods according to the invention are applied to identifying functional sites in a query protein that is very closely related to a particular reference structure, it is likely sufficient to determine the validated functional clusters for that particular reference structure alone, or for any other reference structures that are closely related to that particular reference structure.

Determining Functional Annotation Scores for Putative Functional Reference Clusters and Validated Functional Clusters-9.

The claimed methods represent each validated functional cluster and putative functional reference cluster (referred to in combination as “the training data”) with a functional annotation score. A functional annotation score may be one dimensional or multi-dimensional. A one dimensional functional annotation score refers to a functional annotation score that depends upon one observable. A multi-dimensional functional annotation score refers to a functional annotation score that depends upon one or more observables. Any type of functional annotation score may be used by the claimed methods provided that it creates a separable distribution of training data. A separable distribution of training data refers to the case where the respective functional annotation score distributions for putative functional reference clusters and validated functional clusters are mathematically distinct. As one ordinarily skilled in the art appreciates, since the accuracy of the claimed methods improves as the distinctiveness of these two distributions increases, it is preferable to use functional annotation scores that provide maximally distinct distributions for the putative functional reference clusters and the validated functional clusters.

Since functional clusters are often characterized by evolutionarily conserved residues and concave surface features, functional annotation scores may be selected that relate to either of these two attributes. Functional annotation scores broadly fall into two groups: 1) those functional annotation scores that reflect residue conservation; and 2) those scores that reflect various topographic features, such as the depth, surface area, volume, “mouth” area or “mouth” circumference.

Residue Conservation Based Functional Annotation Scores for Representing Putative Functional Reference Clusters and Validated Functional Clusters.

Each putative functional reference cluster and validated functional cluster may be represented by a distribution of residue conservation scores for its constituent residues. Accordingly, a single or multi-dimensional functional annotation score may be used to characterize each such distribution. Suitable one dimensional functional annotation scores relating to residue conservation include the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

The “cluster maximum residue conservation z-score” refers to the maximum residue conservation z-score of a putative functional reference cluster or a validated functional cluster. The “cluster averaged residue conservation z-score” refers to the mean residue conservation z-score among the residue conservation z-scores that characterize a putative functional reference cluster or a validated functional cluster. The “cluster median residue conservation z-score” refers to the median residue conservation z-score among the residue conservation z-scores that characterize a putative functional reference cluster or a validated functional cluster.

The “cluster maximum neighbor averaged residue conservation z-score” refers to the maximum neighbor averaged residue conservation z-score among the neighbor averaged residue conservation z-scores that characterize a putative functional reference cluster, or a validated functional cluster, and where each neighbor averaged residue conservation z-score is formed by averaging over either first order or second order touching residues. The “cluster averaged neighbor averaged residue conservation z-score” refers to the mean neighbor averaged residue conservation z-score among the neighbor averaged residue conservation z-scores that characterize a putative functional reference cluster, or a validated functional cluster, and where each neighbor averaged residue conservation z-score is formed by averaging over either first order or second order touching residues. The “cluster median neighbor averaged residue conservation z-score” refers to the median neighbor averaged residue conservation z-score among the neighbor averaged residue conservation z-scores that characterize a putative functional reference cluster, or a validated functional cluster, and where each neighbor averaged residue conservation z-score is formed by averaging over either first order or second order touching residues.

The “cluster maximum residue conservation p-score” refers to the maximum residue conservation p-score of a putative functional reference cluster or a validated functional cluster. The “cluster averaged residue conservation p-score” refers to the mean residue conservation p-score among the residue conservation p-scores that characterize a putative functional reference cluster or a validated functional cluster. The “cluster median residue conservation p-score” refers to the median residue conservation p-score among the residue conservation p-scores that characterize a putative functional reference cluster or a validated functional cluster.

The “cluster maximum neighbor averaged residue conservation p-score” refers to the maximum neighbor averaged residue conservation p-score among the neighbor averaged residue conservation p-scores that characterize a putative functional reference cluster, or a validated functional cluster, and where each neighbor averaged residue conservation p-score is formed by averaging over either first order or second order touching residues. The “cluster averaged neighbor averaged residue conservation p-score” refers to the mean neighbor averaged residue conservation p-score among the neighbor averaged residue conservation p-scores that characterize a putative functional reference cluster, or a validated functional cluster, and where each neighbor averaged residue conservation p-score is formed by averaging over either first order or second order touching residues. The “cluster median neighbor averaged residue conservation p-score” refers to the median neighbor averaged residue conservation p-score among the neighbor averaged residue conservation p-scores that characterize a putative functional reference cluster, or a validated functional cluster, and where each neighbor averaged residue conservation p-score is formed by averaging over either first order or second order touching residues.

A residue conservation score distribution may be approximated with the sum of the moments of its distribution. Accordingly, multi-dimensional functional annotation scores may be formed from the moment expansion of a residue conservation distribution. For example, a two dimensional functional annotation score may be formed from the zero moment, which the mean of the distribution, and the first moment, which the variance of the distribution. Still other higher dimension functional annotation scores may be formed by considering higher moments. In addition to expanding a distribution with its moments, a distribution of residue conservation scores may be represented by a plurality of statistical bins where each bin represents a range of residue conservation scores. The occupation count of each bin forms each component of the multi-dimensional functional annotation score. For example if a residue conservation score distribution comprises scores ranging from 1-5, and the distribution is divided into statistical bins with a score width of 0.1, a 50 dimensional functional annotation score may be used to represent the residue conservation score distribution.

Returning to FIG. 5b and the upper most putative functional cluster, the distribution of residue conservation z-scores is {1.3, 1.3, 1.3, 1.4, 1.5, 1.8}. Accordingly, the maximum residue conservation z-score is 1.8, the cluster averaged residue conservation z-score is 1.43, and the median residue conservation z-score, is greater than 1.3 and less than 1.4.

The above discussion further highlights why it is generally preferable at the outset—i.e. even before putative functional reference clusters are identified, to determine residue conservation scores for as many of the reference residues as is computationally practicable. Both the accuracy of putative functional reference cluster identifications and the accuracy of the functional annotation score determinations are increased as the number and density of reference residue conservation scores increases. Still, as one skilled in the art will appreciate, provided that the methods according to the invention are applied to a query structure that is evolutionarily very similar to a particular reference structure, the claimed methods may sufficiently determine residue conservation scores for far less than all or substantially all of the residues that comprise a particular validated functional cluster or putative functional reference cluster. For example, once again assume that each residue on the surface of a reference structure is coordinated by four nearest neighbors. Further assume that a large active site typically contains about 100 residues. Accordingly, even if residue conservation scores are calculated for every tenth residue on the surface of a reference protein the average residue conservation score may not substantially diverge from the average calculated if residue conservation scores had been calculated for all 100 residues.

Topography Based Functional Annotation Scores and the Methods for Determining them.

Many functional clusters are characterized by concave surface features such as grooves, pockets, and clefts on the surface of a protein. Accordingly, a functional annotation score may be based upon one or more topographic observables typical of concave surface features. A functional annotation score based upon a topographic observable is referred to as a topography score. There is no general limitation on the particular topographic observables or the methods of scoring topographic observables that may be used by the methods according to the invention. One set of suitable topographic observables reflect the cluster surface area, cluster volume, cluster depth, cluster “mouth area” and cluster “mouth circumference”.

Either analytical or numerical methods may be used to determine functional topography scores. Numerical methods, such as the various grid based approaches, represent the surface or the sub-surface of a protein within the framework of a three-dimensional lattice of cells. One grid method represents the surface of a protein with a plurality of points and corresponding normal vectors. Via, A., Ferre, F., Brannetti, B., Helmer-Citterich, M., Protein Surface Similarities: A Survey of Methods to Describe and Compare Protein Surfaces, Cell. Mol. Life Sci. 57: 1979-1987 (2000). The shell of points and vectors is then superimposed with a lattice of cubic cells. Each point is then represented by its corresponding cubic face. The volume of a putative functional reference cluster or a validated functional cluster may be determined by summing the volume of the cubic cells that comprise the cluster. The “mouth” area and surface area of a putative functional reference cluster (or validated functional cluster) may be determined by summing the area of the cubic faces that comprise the “mouth” or the surface of the cluster. The “mouth” circumference may be determined by summing the edge lengths of the cubic faces that lie along the circumference of the validated cluster. While grid based methods may be implemented straightforwardly, they are computationally expensive.

Topography scores that characterize a putative functional cluster or validated functional cluster may be analytically determined from the Delaunay tessellation and Alpha shape of a cluster. One Alpha Shape based approach that uses the methods illustrated in FIG. 6, comprises the steps of: 1) determining a three dimensional Delaunay tessellation of all or substantially all of the residues that comprise a putative functional reference cluster (or a validated functional cluster); 2) determining the Alpha Shape of the putative functional reference cluster (or a validated functional cluster) from the Delaunay tessellation; 3) identifying a void with a volume greater than the volume of a solvent molecule from the Delaunay tessellation and the Alpha Shape; and 4) determining a plurality of topography scores for any voids determined in step 3).

Methods for determining the Delaunay tessellation and the Alpha Shape of a putative functional reference cluster were detailed above, in the section entitled, Void Based Methods for Determining a Putative Functional Reference Cluster.

The cluster volume, cluster surface area, cluster “mouth” area, cluster “mouth” circumference, and cluster depth of a putative functional reference cluster or validated functional cluster may be analytically determined from the Delaunay tessellation and the Alpha Shape using the methods detailed in Lang, J., Edelsbrunner, H., Fu, P., Sudhakar, P. V., and Subramaniam, S., Analytical Shape Computation of Macromolecules: Molecular Area and Volume Through Alpha Shape, 33 Proteins, Structure, Function, and Genetics 1-17 (1998) and Measuring Space Filling Diagrams, NCSA Technical Repot 010, (Univ. of Illinois, Urbana Champagne 1993). The corresponding software for determining the surface area and volume may be downloaded at http://www.alphashapes.org/alpha/.

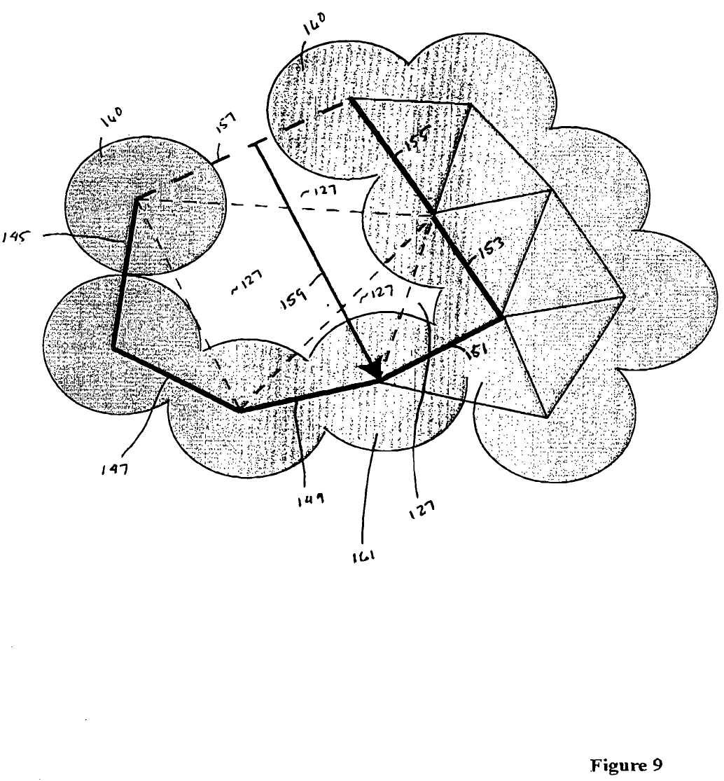

FIG. 9 illustrates the geometric relationships between various topographic quantities, such as the volume, surface area, “mouth” area, “mouth” circumference or depth of a putative functional reference cluster (or a validated functional cluster) and the Delaunay tessellation/Alpha Shape of that putative functional reference cluster. FIG. 9 illustrates the exemplary cluster, first illustrated in FIGS. 7a-c. The Delaunay tessellation of the cluster is shown by the solid and dashed lines. The dashed lines define the empty Delaunay triangles 127 (the two dimensional analogs to the Delaunay tetrahedrons) corresponding to solvent accessible portions of the cluster. The solvent accessible surface area (the two dimensional equivalent of the volume) of the cluster may be determined by summing the areas of the open Delaunay triangles and subtracting the fraction of the atoms contained within the triangles. The cluster arc length (the one dimensional equivalent to the cluster surface area) is defined by summing the lengths of the Delaunay triangle sides 145, 147, 149, 151, 153, 155, shown in bold. The “mouth” size is the length of the dotted edge 157 less the radii of the two atoms 160 that define the “mouth”. The depth of the cluster 159 may be defined: as the maximum distance that may be measured by a vector normal to and originating from the side 157 and terminating at the center of an atom 161 that comprises the putative functional reference cluster; less the radius of that atom 161. While this cluster is illustrated in two dimensions, it is appreciated that all of the topographic features identified in two dimensions have corresponding three dimensional quantities. In three dimensions, the Delaunay triangles correspond to Delaunay tetrahedrons. The solvent accessible cluster area corresponds to the solvent accessible cluster volume. The cluster length corresponds to the cluster surface area. The “mouth” length corresponds to the “mouth” area and the corresponding “mouth” circumference.

Accordingly, the volume of a putative functional reference cluster (or a validated functional cluster) may be determined by summing the volumes of the empty Delaunay tetrahedrons less the fraction of the atomic volumes contained in each tetrahedron. Similarly, the surface area of a functional cluster may be determined by summing the areas of the barrier faces of the barrier tetrahedrons that define the void. The “mouth” area of a cluster, as illustrated, may be determined by summing the areas of the faces of the empty Delaunay tetrahedrons that connect the atoms that ring the “mouth” of a functional cluster. The depth of a functional cluster may be identified with the length of the longest vector that may be determined originating at, and normal to a plane defined by the average position of the atoms that ring the mouth of a functional cluster, and intersecting the center of an atom that comprises the body of the cluster.

In addition to Alpha Shape based approaches for determining the surface area of a cluster, the methods detailed in Zamanakos, G., A Fast and Accurate Analytical Method for the Computation of Solvent Effects in Molecular Simulations, (California Institute of Technology Doctoral Dissertation Publications 2002) and also in Lee, B. and Richards, F. M., The Interpretation of Protein Structures: Estimation of Static Accessibility, J. of Mol. Bio. 55:379-400 (1971) and also Connolly, M., Computation Of Molecular Volume, J. of Amer.Chem. Soc. 107:1118-1124 (1985), may be used by the methods according to the invention.

Composite Functional Annotation Scores.

Since functional clusters are often characterized by both conserved residues and concave surface features, multi-dimensional functional annotation scores may be formed by considering observables relating to residue conservation and topography. By combining the two types of scores, the comparison methods are sensitive to both sequence conservation and fold conservation. A general multi-dimensional functional annotation score reflecting both the sequence conservation and topographic features of training data may be formed by selecting at least two observables selected from the group consisting of: the cluster maximum residue conservation score, the cluster averaged residue conservation score, the cluster median residue conservation score, neighbor averaged quantities of any of the foregoing, the z-score or p-scores of any of the foregoing, the n'th moment of a cluster's residue conservation score distribution, the cluster surface area, the cluster depth, the cluster volume, the cluster “mouth” area, and the cluster “mouth” circumference. One embodiment on the invention uses a four dimensional functional annotation score to represent the training data formed from the: 1) the cluster maximum residue conservation z-score; 2) the cluster volume; 3) cluster depth and 4) cluster “mouth” area.

Identifying a First Sub-Set of Functional Annotation Scores that Represents the Putative Functional Clusters, and a Second Sub-Set of Functional Annotation Scores that Represents the Validated Functional Clusters-11.

The inventors observed that when putative functional reference clusters and validated functional clusters are both represented by a four dimensional functional annotation score formed from the: 1) maximum residue conservation z-score; 2) cluster volume; 3) cluster depth; and 4) cluster “mouth” area, the two distributions overlapped each other. Determining a first sub-set of functional annotation scores that represents the putative functional reference clusters and a second sub-set that represents the validated functional clusters reduces to the well known binary classification problem in statistics, or equivalently, the supervised learning problem in pattern recognition. The binary classification problem asks: given a set of training objects characterized by one or more observables, and wherein each object is assigned to one of two groups, and given a new object characterized by the same observables, which of the two classes should the new object be assigned to.

In the instant case, the training set consists of putative functional reference clusters and validated functional clusters. The testing set is one or more putative functional clusters on the surface of one or more query proteins. The solutions to this binary classification problem define a hyperplane that divides the vector space—i.e. the functional annotation score space-used to represent the putative functional reference clusters, and validated functional clusters into two half-spaces. One half-space represents the functional annotation scores that tend to characterize putative functional reference clusters and the second half-space represents the functional annotation scores that tend to characterize validated functional clusters. A putative functional cluster is then assigned to one of these two classes (vector spaces) based upon the functional annotation scores used to represent it.

One method that may be used to solve the present binary classification problem uses a Support Vector Machine (“SVM”). See also, Napnik, V. N., The Nature of Statistical Learning Theory, (Springer Verlag 1995). Since SVM programs and methods are readily available and well known in the art, the foregoing discussion provides a qualitative discussion of the application of SVMs for functional cluster identifications. However, the upcoming section titled, Methods for Determining a Continuous SVM Score, provides a formal mathematical framework for determining optimal SVM hyperplanes and classifying functions for both linear, and non-linear SVMs, including “soft margin” formulations from the training data.

SVMs represents each object in the training set and the testing set as a vector of real numbers. Linear SVMs find a hyperplane that divides the functional annotation score space used to represent the training data into two half spaces. Non-linear SVMs first map the training space into a higher dimensional space using a kernel function, K, and then divide this higher dimensional space into two half spaces. The testing data is then mapped into one of the two half spaces to determine which class(es) the testing data is assigned to. SVMs output a score, referred to herein as a SVM score for each object in the test set. SVM scores are binary scores, usually −1 and +1, where +1 corresponds to one class and −1 corresponds to the other class. For training data that is not linearly separable due to misclassified training data points or noise, the SVM methods may use the “soft-margin” techniques that are known in the art. Cortes, C. and Vapnik, V. (1995). Support vector networks. Machine Learning, 20:1-25.

In one embodiment of the invention that uses an SVM, the training data, comprising putative functional reference clusters and validated functional clusters are represented by a four dimensional vector in the space formed from the: 1) cluster mouth area; 2) cluster depth; 3) cluster volume; and 4) the maximum residue conservation z-score in the cluster. The testing data—i.e. putative functional clusters are represented in the same four dimensional vector space. However, as was detailed in the earlier sections, any functional annotation score may be used to represent training and testing data provided that the training data is separable.

Suitable kernels include the Radial Basis Function (“RBF”) kernel, K(x₁,x₂)=exp(−γ∥x₁−x₂∥²), γ>0, the Polynomial Basis Kernel, K(x₁, x₂)=(γx₁^{T}x₂+r)^{d} or the Sigmoid Basis Kernel K(x₁, x₂)=tan h(k(x₁·x₂)+Θ) where x₂ is the map of the training datum x₁. γ, r, d, κ and Θ are kernel parameters. One embodiment of the invention uses the RBF kernel with “soft margin” classification. This kernel was selected because: 1) many of the functional annotation scoring functions nonlinearly correlate between the two classes (validated functional clusters and putative functional clusters); 2) others have shown that the linear kernel and the Sigmoid Kernel behave like the RBF kernel for certain values of γ; and 3) it has less numerical difficulties—e.g. singularities and infinities.

Using the RBF kernel with “soft-margin” classification requires setting two parameters: C, the penalty parameter of the “soft margin” error term and γ. If values other than the default values are used, C and γ must be determined by modeling the training accuracy for varying values of C and γ. One method that may be suitably employed is to separate the training data into two groups: a first group for training data and a second group for testing the prediction of the model based upon varying values of C and γ. Since the second group of testing data is actually known, the values of C and γ may be optimized accordingly. Values for C and γ may be determined through a two dimensional “grid search”—i.e. an exhaustive search of the two dimensional parameter space formed by C and γ-or through use of search heuristics. Hsu, C. W, Chang, C. C., Lin, C. J., A Practical Guide to Support Vector Classification, available at http://216.239.33.104/search?g=cache:kFYtzIS8OJkJ:www.csie.ntu.edu.tw/˜cjlin/papers/guide/guide.pdf+practical+guide+to+support+vector+classification&h1=en&ie=UTF-8 discusses in detail the implementation of SVM for solving binary classification problems.

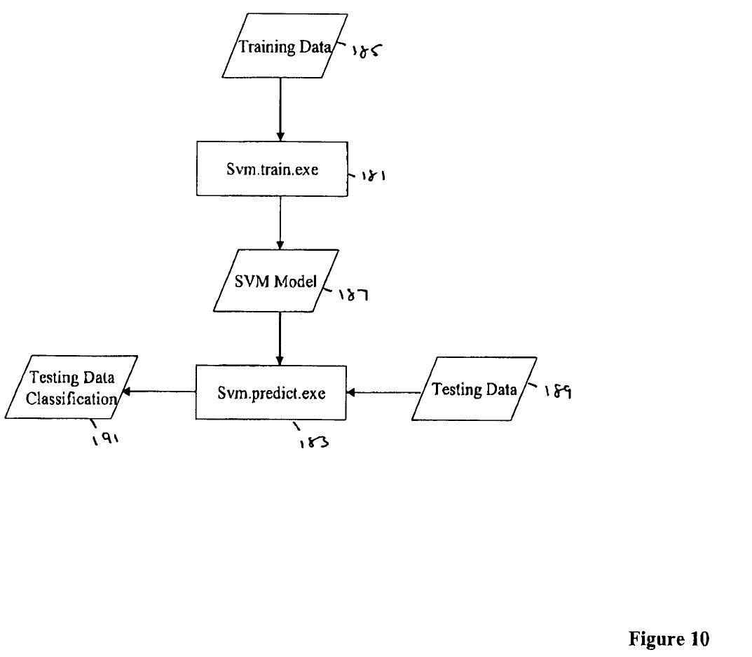

One method according to the invention for determining a functional annotation is based upon the “soft-margin” SVM developed by Chih-Wei Hsu, Chiu-Chungs Chang and Chih-Jen Lin (“Lin's SVM”). Lin's SVM is available for download at http://www.csie.ntu.edu.tw/˜cjlin/. In addition to the source code for Lin's SVM, provided both in C++ and Java, the LIBSVM package includes two examples demonstrating the use of LIBSVM, a README file detailing the use of Lin's SVM, and a precompiled Java class archive. Lin's SVM program comprises five files: 1) Svm.cpp; Svm.header.c; Svm.train.c; Svm.predict.c, and Svm.output.c. When these five files are compiled, three executable files are generated: svm-train.exe, svm-predict.exe, and svm-scale.exe. FIG. 10 illustrates the data architecture between svm-train.exe 181, svm-predict,exe 183, and the input and output data to these two executables. Svm-scale.exe scales training data attributes—i.e. training vector components, to a new range [a,b], where a,b ε Rⁿ. Preferred scaled training data attributes range from [−1, +1] or [0,+1]. Training data 185 represented in vector form is input to svm-train.exe 181. Once executed, smv-train.exe 181 outputs the SVM hyperplane model 187. This output 187, and a testing datum 189, represented as a vector, are then input to svm-predict.exe 183. Svm.predict.exe outputs the classification [−1, +1] 191 of the testing datum based upon the training data. Other suitable SVMs that may be download include Thorsten Joachims SVM available for download at http://svmlight.joachims.org/.

Table 1 illustrates the application of Lin's SVM to a hypothetical set of 16 putative functional clusters each characterized by a cluster averaged residue conservation z-score. The training data comprises putative functional reference clusters and eight validated functional cluster. Putative functional reference clusters are identified as (−1) and validated functional clusters are identified as (1). Each training datum is also characterized by a cluster averaged residue conservation z-score The source code was compiled using the gcc compiler v. 3.2 that is included with Red Hat, Inc.'s Linux (v. 8.0) (Durham, N.C.).

| Cluster | Training Data | | Testing Data | |
|---|---|---|---|---|
| No. | Class | Res. Cons. z-score | Res. Cons. z-score | SVM Score |
| 1 | 1 | 3.8800 | 3.5013 | 1 |
| 2 | 1 | 3.1926 | 2.8886 | 1 |
| 3 | 1 | 2.4611 | 2.2951 | 1 |
| 4 | 1 | 1.8972 | 1.4903 | 1 |
| 5 | 1 | 1.0973 | 0.6050 | 1 |
| 6 | 1 | 0.2120 | 0.2082 | 1 |
| 7 | 1 | 0.1852 | 0.1504 | 1 |
| 8 | 1 | 0.1463 | 0.0615 | 1 |
| 9 | −1 | 0.0235 | −0.1888 | −1 |
| 10 | −1 | −0.4035 | 0.3492 | 1 |
| 11 | −1 | 0.6231 | −0.0103 | −1 |
| 12 | −1 | 0.0156 | −0.7984 | −1 |
| 13 | −1 | −0.4321 | −1.6074 | −1 |
| 14 | −1 | −1.2098 | −2.4567 | −1 |
| 15 | −1 | −2.0124 | −3.1987 | −1 |
| 16 | −1 | −2.8065 | −4.0436 | −1 |

Table 1 illustrates the application of an SVM for determining the class of 16 putative functional clusters each characterized by a cluster averaged residue conservation z-score based upon a set of training data comprising 8 putative functional clusters and 8 validated functional clusters and wherein each training datum is characterized by a cluster averaged residue conservation z-score.

In addition to the SVM based approaches, other suitable binary classifications algorithms known in the art include, the Linear/Quadratic Logistic Discriminant methods, Bayesian methods, the K-nearest neighbors method, decision tree methods, neural network methods, and stochastic methods. Duda, R. V., Hart, P. E., and Stork, D. G., Pattern Classification, (Wiley Interscience 1982).

Determining a Plurality of Residue Conservation Scores for a Query Protein-13.

Residue conservation scores on the surface of a query protein are determined in order to identify putative functional clusters on the surface of a query protein. The same methods which were detailed in the section, entitled, Determining Residue Conservation Scores for a Plurality of Reference Residues from at Least One Reference Protein 1, may be used for determining residue conservation scores for a plurality of the residues on the surface of a query protein. In general, as was detailed in this corresponding section, the accuracy of the claimed methods increases as the number of residue conservation scores on the surface of a query structure increases. Still, at the cost of sensitivity for smaller functional sites, the claimed methods may sufficiently determine far less than all or substantially all of the residue conservation scores for a particular query protein. For example, consider the case of a query protein comprising a large functional site of 100 residues. If the residue conservation scores are determined for only one of out of every ten query residues, a putative functional cluster of ten high scoring residues may still be identified.

Determining a Plurality of Surface Orientation Scores for a Query Protein-15.

Surface orientation scores are determined in order to identify putative functional clusters on the surface of a query protein. The same methods which were detailed above in the section, entitled, Determining a Plurality of Surface Orientation Scores for at Least One Reference Protein 3, may be used for determining for a plurality of surface orientation scores for a query protein. In general, as was detailed in this earlier section, the accuracy of the claimed methods increases as the number and density of surface orientation scores increases across a query structure.

Determining at Least One Putative Functional Cluster on the Surface of a Query Protein-17.

The claimed methods use putative functional clusters as testing data within a binary classification model. More particularly, the functional annotation scores that characterize putative functional clusters are mapped into one of the two half spaces that represent the training data. The claimed methods also use putative functional clusters, outside of a binary classification model, for the purpose of identifying a cluster of residues on the surface of a query protein that is to be tested for the likelihood of its biological function. The same methods that were detailed in the section above, entitled, Methods for Determining at Least One Putative Functional Reference Cluster on the Surface of at Least One Reference Protein 5, may be used for determining at least one putative functional cluster on the surface of the query protein 17.

Determining a Functional Annotation Score for a Putative Functional Cluster on the Surface of a Query Protein-19.

The same methods that were in the section above, entitled, Determining Functional Annotation Scores for Putative Functional Reference Clusters and Validated Functional Clusters 9, are applicable to determining one or more functional annotation scores for a putative functional cluster. One embodiment of the invention represents putative functional clusters with: 1) the maximum residue conservation z-score; 2) the cluster depth; 3) the cluster surface area; and 4) the cluster “mouth” area. As one ordinarily skilled in the art would appreciate, it is preferable that the same type of functional annotation scores that are used to represent the training data be used to represent putative functional clusters.

Determining Whether a Putative Functional Cluster is a Functional Cluster or a Non-Functional Cluster-21.

A putative functional cluster is tested to determine whether it is a functional cluster or a non functional cluster by comparing its functional annotation score to the two sets of functional annotation scores that characterize the two classes of training data. In one embodiment of the invention that uses the SVM algorithm to classify the training data, an SVM maps a vector that represents a putative functional cluster into the higher dimensional space used to represent, and bifurcate, the training data. If a putative functional cluster maps into the half space corresponding to the putative functional reference clusters it is annotated as a non functional cluster; if it maps into the half space corresponding to the validated functional clusters, it is annotated as a functional cluster.

Methods for Determining a Continuous SVM Score for a Putative Functional Cluster-22.

Another aspect of the invention is a method for determining a continuous SVM score. As used herein a continuous SVM score refers to a score scales with the distance between the optimal SVM surface and a point in the functional annotation score space that represents a testing datum—i.e. the functional annotation score of a putative functional cluster. Continuous SVM scores are a preferred class of functional annotation scores for representing putative functional clusters within the methods according to the invention for determining the probability that a putative functional cluster is in fact functional.

One embodiment of the invention identifies a continuous SVM score with the minimum distance between a testing datum point and an SVM hyperplane. In order to illustrate how such distances may be calculated, it is first necessary to detail the relationship between the training data vectors and the selection of the SVM hyperplane.

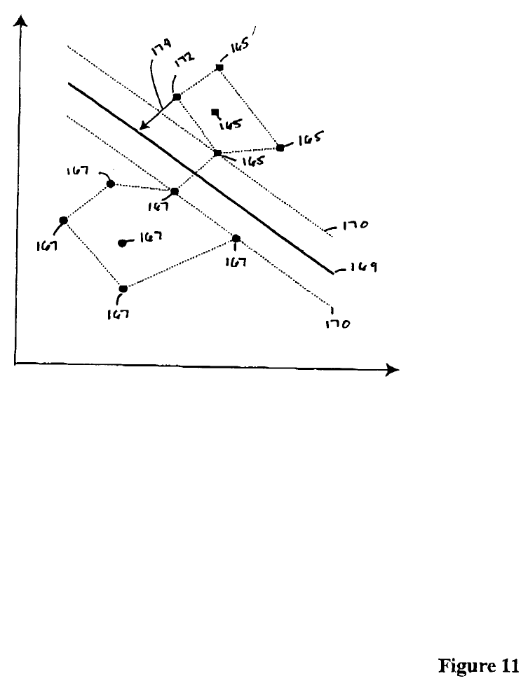

Referring to FIG. 11, SVM's determine the optimal hyperplane (w^{T}x)+b=0 that maximally separates two classes of training data {(x₁, y₁)}165, 167. More particularly, the hyperplane 169 is orthogonal to, and bisects, the distance ρ 171 connecting the respective convex hulls 173, 175 of the two training data classes 165, 167.

SVM theory assumes that the training data {(x₁,y₁)}, where x₁ ε R^{N}, may be represented by the following constraints:
w^{T}x₁+b≧1 if yᵢ=1 and Eq. 1
w^{T}xᵢ+b≦−1 if yᵢ=−1
The classifying function corresponding to the optimal SVM hyperplane is given by f(x)=sign (w^{T}x+b). The support vectors may be represented as {xᵢ|yᵢ(w^{T}x₁+b)=1}170. It may be shown that for any arbitrary training or testing datum xₖ 172, the minimum distance 174 between xₖ 172 and the hyperplane 169 is given by

$$r ( {x}_{k} , w ) = \frac{{w}^{T} {x}_{k} + b}{\vert w \vert} . \qquad Eq . 2$$

It follows from the definition of the support vectors and Equation 2 that

$$ρ = \frac{2}{\vert w \vert} 171.$$

Thus, the problem finding a continuous SVM score according to Equation 2 reduces to finding w and b to finding w and b such that

$$ρ = \frac{2}{\vert w \vert} 171$$

is maximized for all {(xᵢ,yᵢ)} subject to the constraints of Eq. 1. Equivalently, if a new function Φ(w)=½w^{T}w is defined, w and b may be found by minimizing Φ(w)=½w^{T}w for all {(xᵢ,yᵢ)} subject to yᵢ(w^{T}xᵢ+b)≧1. Methods of solving constrained quadratic optimizations problems are well-known in mathematics. The solutions of w and b may be shown to have the form:

$$w = {\sum }_{i}^{} {α}_{i} {y}_{i} {x}_{i} and \qquad Eq . 3$$

b=yₖ−w^{T}xₖ for any xₖ such that αₖ≠0
and the optimal SVM classifying function has the form:

$$f ( x ) = sign ( {\sum }_{i}^{} {α}_{i} {y}_{i} {x}_{i}^{T} x + b ) \qquad Eq . 4$$

If the training data is not linearly separable, slack variables ξᵢ may be used to allow for the “soft margin” classification of difficult, or noisy training data. In this case, w and b are found by minimizing

$$Φ ( w ) = 1 / 2 {w}^{T} w + C {\sum }_{i}^{} {ξ}_{i}$$

for all {(xᵢ, yᵢ)} subject to yᵢ(w^{T}xᵢ+b)≧1−ξᵢ. The parameter C controls the overfitting of data. The solutions to this minimization problem are given by:

$$w = {\sum }_{i}^{} {α}_{1} {y}_{1} {x}_{i} and \qquad Eq . 5$$

b=yₖ(1−ξₖ)−w^{T}xₖ where k=arg max αₖ
Thus, the SVM “soft margin” classifying function may be represented by

$$f ( x ) = sign ( {\sum }_{i}^{} {α}_{i} {y}_{i} {x}_{i}^{T} x + {b}_{sm} ) \qquad Eq . 6$$

The methods according to the invention may use both linear and non-linear SVMs. Nonlinear SVMs map the training data into a higher dimensional feature space, F, via a nonlinear map Φ: Rⁿ→F and then performs the above linear algorithm in F. It may be shown that the nonlinear SVM classifying function may be represented as

$$f ( x ) = sign ( {\sum }_{i = 1}^{} {v}_{i} \cdot K ( {x}_{1} , x ) + b ) \qquad Eq . 7$$

where K(xᵢ, x)=(Φ(xᵢ)^{T}Φ(x)) and vᵢ=αᵢyᵢ. In many cases, evaluating these dot products will be very computationally expensive, but certain kernel functions K(xᵢ, x) allow for very efficient evaluation of the dot products in F.

One popular kernel with SVM practitioners is the Polynomial Kernel, K(x₁, X₂)=(x^{T}x₂)^{d} where d is the dimensionality of the feature space, F. Other common kernels are the Radial Basis Function Kernel K(x₁,x₂)=exp(−∥x₁−x₂∥²γ) and the Sigmoid Kernels K(x₁, X₂)=tan h(k(x₁·x₂)+Θ). In general, for every kernel that gives rise to a positive matrix (k(x₁, x₂ ))_{i,j} a map Φ may be constructed such that k(x₁, x₂)=(Φ(x₁)^{T}Φ(x₂)) holds.

The foregoing mathematics provides the necessary mathematical machinery for calculating a continuous SVM score according to Equation 2. The idea of using the distance between the optimal SVM hyperplane and a testing datum as a scoring function is based upon the assumption that the confidence of a correct SVM classification should be a monotonic increasing function of the distance between a testing datum x and SVM hyperplane f(x). Accordingly, any monotonic function f(r(x,w)) of the distance between the optimal SVM hyperplane and a testing datum, may be used as a continuous SVM score.

Another method according to the invention identifies a continuous SVM score with

$$svm - cont = {\sum }_{i = 1}^{} {v}_{i} \cdot K ( {x}_{i} , x ) \qquad Eq . 8$$

Equation 8 is a positive or negative number that monotonically scales with the distance between the testing datum x and the optimal SVM hyperplane.

Computer programs for determining a continuous SVM score may be developed by modifying existing SVMs to calculate Equation 2 or Equation 8. Such modifications are well within the capacity of one ordinarily skilled in the art. One method according to the invention for determining a continuous SVM score is based upon modifying Lin's SVM to calculate Equation 2 or Equation 8. Lin's SVM is available for download at http://www.csie.ntu.edu.tw/˜cilin/. Svm.ccp is the only file that must be modified to calculate Equation 8.

Table 2 illustrates the application of the method according to the invention for determining a continuous SVM score according to Equation 8 to an exemplary set of putative functional clusters that are each characterized by a cluster averaged residue conservation z-score. Putative functional reference clusters are identified as (−1) and validated functional clusters are identified as (1). Svm.cpp was modified to calculate a continuous SVM score according to Equation 8. The source code was compiled using the gcc compiler v.3.2 that is included with Red Hat, Inc.'s Linux (v.8.0) (Durham, N.C.).

| | |
|---|---|
| Training Data | Testing Data |

| Cluster | | Res. Cons. | Res. Cons. | Cont. SVM |
|---|---|---|---|---|
| No. | Class | z-score | z-score | Score |
| 1 | 1 | 3.8800 | 3.5013 | 1.029814 |
| 2 | 1 | 3.1926 | 2.8886 | 0.988739 |
| 3 | 1 | 2.4611 | 2.2951 | 1.003771 |
| 4 | 1 | 1.8972 | 1.4903 | 0.988741 |
| 5 | 1 | 1.0973 | 0.6050 | 1.045351 |
| 6 | 1 | 0.2120 | 0.2082 | 1.009442 |
| 7 | 1 | 0.1852 | 0.1504 | 0.983124 |
| 8 | 1 | 0.1463 | 0.0615 | 0.927896 |
| 9 | −1 | 0.0235 | −0.1888 | 0.668931 |
| 10 | −1 | −0.4035 | 0.3492 | 1.046231 |
| 11 | −1 | 0.6231 | −0.0103 | 0.869376 |
| 12 | −1 | 0.0156 | −0.7984 | −0.366107 |
| 13 | −1 | −0.4321 | −1.6074 | −1.030156 |
| 14 | −1 | −1.2098 | −2.4567 | −0.991128 |
| 15 | −1 | −2.0124 | −3.1987 | −0.986953 |
| 16 | −1 | −2.8065 | −4.0436 | −1.037789 |

Table 2 illustrates the application of the method according to the invention for determining a continuous SVM score according to Equation 8 to an exemplary set of putative functional clusters that are each characterized by a cluster averaged residue conservation z-score.

### EXAMPLE

### Identifying the Functional Site on PDB:12asA and Determining its Corresponding Continuous SVM Score

The method illustrated in FIGS. 1b and 4 were implemented in JAVA JRE 1.3 to identify a putative functional cluster corresponding to the small molecule binding site on the surface of PDB:12asA and determine its corresponding continuous SVM score. The next section will illustrate a method for determining the probability that the putative functional cluster identified in this section is correct based upon its continuous SVM score.

The reference structure set was formed by selecting those co-crystal structures listed in the PDB Select database that are X-ray crystal structures, and only bound to small molecules—i.e. not bound to polynucleotide structures. The PDB Select database contains PDB identification numbers; it may be downloaded at http://www.cmbi.kun.nl/gv/pdbsel/. The relevant structure files may be selected from the PDB Select database by hand curation or though the use of an automated script. No residue substitutions or side chain replacements were made to reference structure set.

A set of homologous template sequences to each reference sequence was determined by using PSI-BLAST and the NCBI Protein Database. All the default values in PSI-BLAST were used except, −h=5×10⁻⁴ and −e=1×10⁻³ where −h is the step e-value and -e is the final e-value. Preferred template sequences were prepared using the HSSP threshold function, where v=0. Residue conservation z-scores were determined from a multiple sequence alignment of each reference sequence with the preferred template sequences using the Shannon Entropy scoring function illustrated in FIG. 2b. The multiple sequence alignment was generated using Clustal W (v.1.8). All the default values in Clustal W were used. Each residue conservation z-score was averaged over the residue conservation z-scores corresponding to first order ‘touching’ neighbor residues. Threshold distances for two residues to be identified as touching were based upon the van der Waals radii of their constituent heavy atoms and the van der Waals radius of a water molecule.

Surface orientation scores were determined for each reference residue using the vector dot-product method detailed in FIG. 3 based upon the relative geometry between a reference residue and its first order touching residues.

Putative functional reference clusters were identified using the methods illustrated in FIGS. 4 and 5. Surface orientation scores were distributed in 0.05 width bins ranging from zero to one. The putative functional residue limit was determined by: 1) identifying the bin with largest number of surface orientation scores greater than 0.4; and 2) determining the number of surface orientation scores in such a bin. Initial surface orientation and residue conservation z-score threshold limits were 0.4 and 0.5 respectively. Surface orientation and residue conservation threshold limits were increased in 0.05 and 0.1 increments respectively.

Validated functional clusters were determined directly from the co-crystal structures.

Mouth “area”, void depth and void volume were determined for each putative functional cluster and validated functional cluster based upon the Alpha Shape based methods illustrated in FIG. 6 and further detailed in FIGS. 7a-c and FIG. 9, and implemented in JAVA, JRE 1.3. Before determining these quantities the surface of each cluster was “smoothed” by removing those solvent accessible heavy atoms that were “touched” by less than 10 first order touching neighbors. An Alpha Shape value of 1.4 was used.

Each putative functional reference cluster and each validated functional cluster was represented by a four dimensional functional annotation score vector comprising four components: 1) the maximum neighbor averaged residue conservation z-score found in the cluster (either putative functional reference cluster or validated functional cluster); 2) the cluster's “mouth” area; 3) the cluster's void depth; and 4) the cluster's void volume.

The same methods that were used to identify putative functional reference clusters were used to identify putative functional clusters. Each putative functional cluster was represented in the same functional annotation score space as was used to represent the putative functional reference clusters and the validated functional clusters.

Since PDB:12asA is a homodimer, residue conservation scores were only determined for one of the two identical chains.

A modified version of Lin's SVM was used to determine a continuous SVM score according to Equation 8 for each putative functional cluster. The source code was compiled using the gcc compiler (v.3.2) that is included with Red Hat, Inc.'s Linux (v.8.0) (Durham, N.C.). Each putative functional reference cluster was assigned a “−1” within the SVM model; each validated functional cluster was assigned a “+1” functional cluster within the SVM. The Radial Basis Function Kernel was used with γ=1. The input vector components were not scaled.

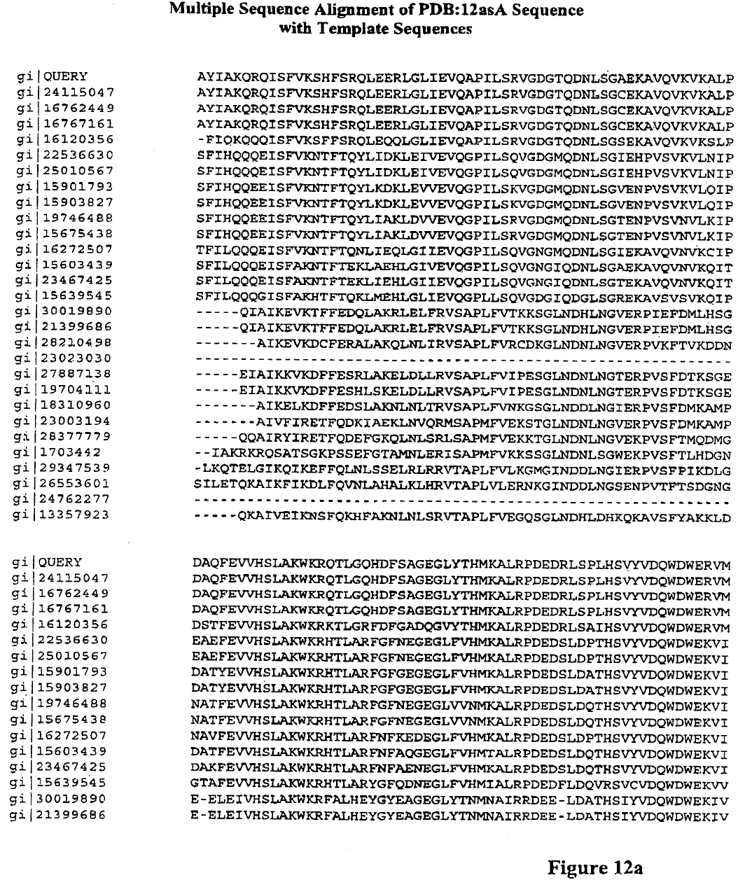

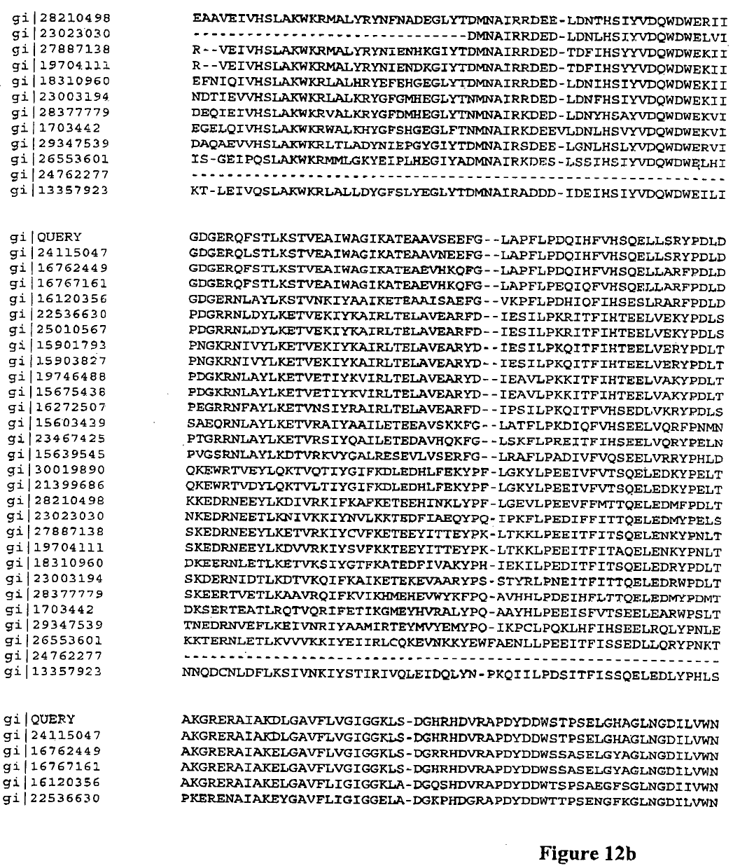

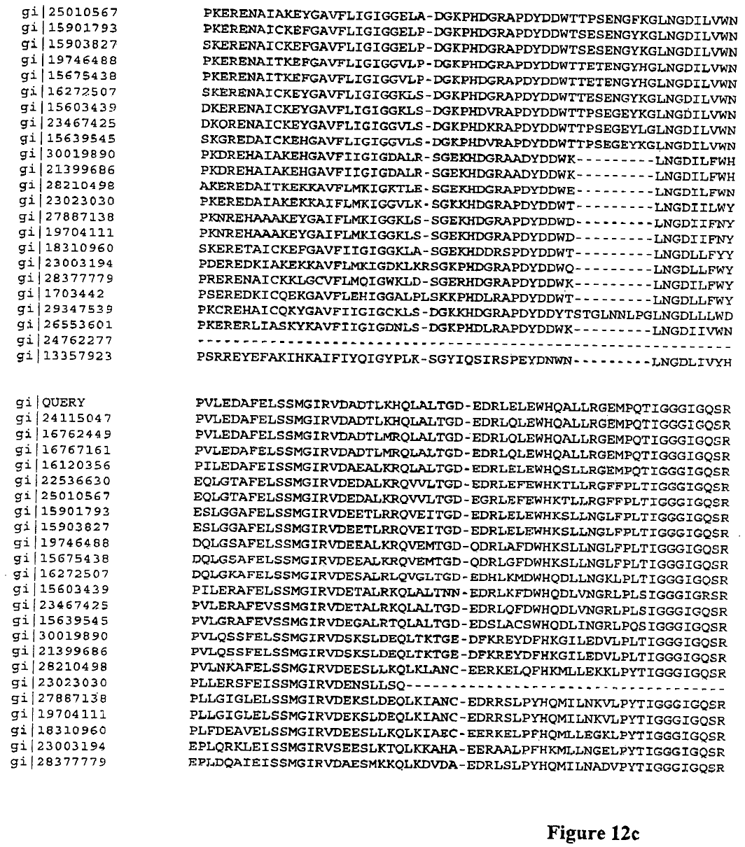

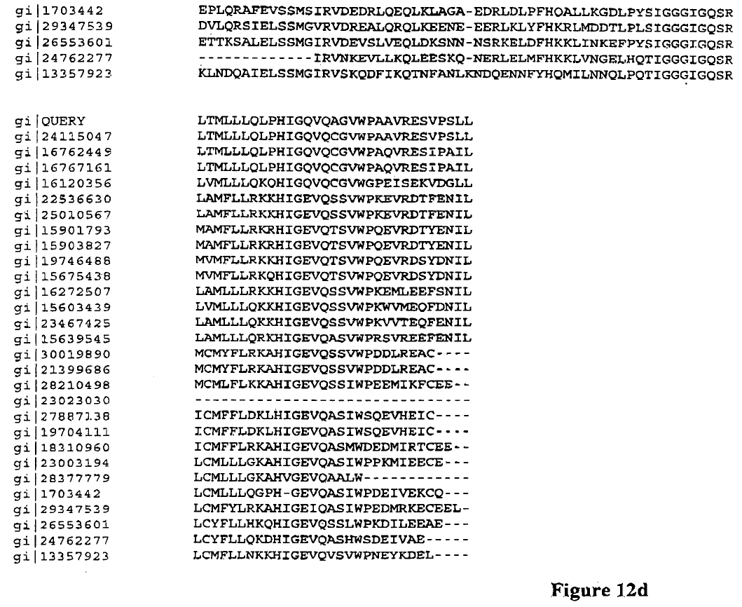

FIGS. 12a-d illustrate the multiple sequence alignment formed between the alpha chain of PDB:12asA and 28 template sequences. Next to each template sequence is its corresponding NCBI gi identification number. Because each sequence in the multiple sequence alignment exceeds the width of a page, the multiple sequence alignment is shown “wrapping” down the page and continuing to the next Figure.

Table 3 lists for each query residue: 1) its type and identification number listed under the column headed, “Residue”; 2) a raw residue conservation score listed under the column headed, “Raw”; and 3) the z-score the of raw residue conservation score under the column headed, “Z-score”. Each residue is numbered in a format according to:
Residue Type PDB Residue Number|Adjusted Residue Number

The PDB Residue Number refers to the residue number listed in the PDB record. Since PDB records do not always begin residue numbering at one, the Adjusted Residue Number refers to the residue number within a second residue numbering scheme beginning at one.

| Residue | Raw Cons. | |
|---|---|---|
| Id. | Score | Res. Cons. z-core |
| ALA4\|1: | 0.7968 | −1.2730 |
| TYR5\|2: | 0.7511 | −1.0121 |
| ILE6\|3: | 0.7509 | −1.0110 |
| ALA7\|4: | 0.7787 | −1.1695 |
| LYS8\|5: | 0.7722 | −1.1327 |
| GLN9\|6: | 0.6553 | −0.4644 |
| ARG10\|7: | 0.7072 | −0.7610 |
| GLN11\|8: | 0.6809 | −0.6105 |
| ILE12\|9: | 0.4850 | 0.5086 |
| SER13\|10: | 0.7181 | −0.8234 |
| PHE14\|11: | 0.7200 | −0.8343 |
| VAL15\|12: | 0.6743 | −0.5732 |
| LYS16\|13: | 0.5519 | 0.1263 |
| SER17\|14: | 0.7981 | −1.2804 |
| HIS18\|15: | 0.7973 | −1.2758 |
| PHE19\|16: | 0.4674 | 0.6096 |
| SER20\|17: | 0.7414 | −0.9565 |
| ARG21\|18: | 0.8291 | −1.4579 |
| GLN22\|19: | 0.8413 | −1.5277 |
| LEU23\|20: | 0.5598 | 0.0812 |
| GLU24\|21: | 0.7887 | −1.2271 |
| GLU25\|22: | 0.7688 | −1.1133 |
| ARG26\|23: | 0.8251 | −1.4347 |
| LEU27\|24: | 0.4632 | 0.6332 |
| GLY28\|25: | 0.7653 | −1.0929 |
| LEU29\|26: | 0.5745 | −0.0028 |
| ILE30\|27: | 0.8437 | −1.5411 |
| GLU31\|28: | 0.5994 | −0.1452 |
| VAL32\|29: | 0.5454 | 0.1638 |
| GLN33\|30: | 0.6765 | −0.5858 |
| ALA34\|31: | 0.5500 | 0.1375 |
| PRO35\|32: | 0.3995 | 0.9972 |
| ILE36\|33: | 0.6372 | −0.3613 |
| LEU37\|34: | 0.6103 | −0.2075 |
| SER38\|35: | 0.6321 | −0.3317 |
| ARG39\|36: | 0.8143 | −1.3731 |
| VAL40\|37: | 0.7393 | −0.9443 |
| GLY41\|38: | 0.7413 | −0.9561 |
| ASP42\|39: | 0.7708 | −1.1246 |
| GLY43\|40: | 0.3956 | 1.0195 |
| THR44\|41: | 0.7069 | −0.7596 |
| GLN45\|42: | 0.6052 | −0.1780 |
| ASP46\|43: | 0.4150 | 0.9089 |
| ASN47\|44: | 0.6453 | −0.4071 |
| LEU48\|45: | 0.4157 | 0.9045 |
| SER49\|46: | 0.6411 | −0.3831 |
| GLY50\|47: | 0.4939 | 0.4579 |
| ALA51\|48: | 0.8536 | −1.5975 |
| GLU52\|49: | 0.4978 | 0.4356 |
| LYS53\|50: | 0.6705 | −0.5512 |
| ALA54\|51: | 0.5889 | −0.0849 |
| VAL55\|52: | 0.4718 | 0.5844 |
| GLN56\|53: | 0.6881 | −0.6520 |
| VAL57\|54: | 0.5933 | −0.1100 |
| LYS58\|55: | 0.8059 | −1.3252 |
| VAL59\|56: | 0.7423 | −0.9618 |
| LYS60\|57: | 0.6657 | −0.5238 |
| ALA61\|58: | 0.8369 | −1.5023 |
| LEU62\|59: | 0.7985 | −1.2827 |
| PRO63\|60: | 0.7472 | −0.9896 |
| ASP64\|61: | 0.7666 | −1.1003 |
| ALA65\|62: | 0.8101 | −1.3490 |
| GLN66\|63: | 0.8117 | −1.3584 |
| PHE67\|64: | 0.7731 | −1.1378 |
| GLU68\|65: | 0.5076 | 0.3798 |
| VAL69\|66: | 0.5628 | 0.0642 |
| VAL70\|67: | 0.4822 | 0.5245 |
| HIS71\|68: | 0.5377 | 0.2076 |
| SER72\|69: | 0.4286 | 0.8313 |
| LEU73\|70: | 0.4157 | 0.9045 |
| ALA74\|71: | 0.4236 | 0.8596 |
| LYS75\|72: | 0.4264 | 0.8434 |
| TRP76\|73: | 0.4026 | 0.9794 |
| LYS77\|74: | 0.4264 | 0.8434 |
| ARG78\|75: | 0.4183 | 0.8897 |
| GLN79\|76: | 0.8310 | −1.4689 |
| THR80\|77: | 0.6342 | −0.3437 |
| LEU81\|78: | 0.4157 | 0.9045 |
| GLY82\|79: | 0.7985 | −1.2826 |
| GLN83\|80: | 0.7352 | −0.9210 |
| HIS84\|81: | 0.6236 | −0.2832 |
| ASP85\|82: | 0.7035 | −0.7402 |
| PHE86\|83: | 0.5822 | −0.0467 |
| SER87\|84: | 0.8189 | −1.3993 |
| ALA88\|85: | 0.8351 | −1.4920 |
| GLY89\|86: | 0.7283 | −0.8818 |
| GLU90\|87: | 0.5650 | 0.0514 |
| GLY91\|88: | 0.3956 | 1.0195 |
| LEU92\|89: | 0.5360 | 0.2176 |
| TYR93\|90: | 0.5746 | −0.0033 |
| THR94\|91: | 0.6015 | −0.1573 |
| HIS95\|92: | 0.6337 | −0.3408 |
| MET96\|93: | 0.3653 | 1.1930 |
| LYS97\|94: | 0.5867 | −0.0722 |
| ALA98\|95: | 0.3474 | 1.2953 |
| LEU99\|96: | 0.5073 | 0.3815 |
| ARG100\|97: | 0.3417 | 1.3279 |
| PRO101\|98: | 0.6868 | −0.6445 |
| ASP102\|99: | 0.3380 | 1.3488 |
| GLU103\|100: | 0.4165 | 0.9003 |
| ASP104\|101: | 0.5159 | 0.3319 |
| ARG105\|102: | 0.9055 | −1.8942 |
| LEU106\|103: | 0.4559 | 0.6749 |
| SER107\|104: | 0.5426 | 0.1794 |
| PRO108\|105: | 0.7686 | −1.1117 |
| LEU109\|106: | 0.7007 | −0.7241 |
| HIS110\|107: | 0.4052 | 0.9647 |
| SER111\|108: | 0.3527 | 1.2650 |
| VAL112\|109: | 0.6182 | −0.2525 |
| TYR113\|110: | 0.3946 | 1.0256 |
| VAL114\|111: | 0.3509 | 1.2753 |
| ASP115\|112: | 0.3380 | 1.3488 |
| GLN116\|113: | 0.3584 | 1.2323 |
| TRP117\|114: | 0.3248 | 1.4243 |
| ASP118\|115: | 0.3380 | 1.3488 |
| TRP119\|116: | 0.3248 | 1.4243 |
| GLU120\|117: | 0.3478 | 1.2929 |
| ARG121\|118: | 0.6374 | −0.3621 |
| VAL122\|119: | 0.5828 | −0.0502 |
| MET123\|120: | 0.5349 | 0.2235 |
| GLY124\|121: | 0.8171 | −1.3892 |
| ASP125\|122: | 0.6884 | −0.6539 |
| GLY126\|123: | 0.6844 | −0.6309 |
| GLU127\|124: | 0.7129 | −0.7935 |
| ARG128\|125: | 0.4282 | 0.8331 |
| GLN129\|126: | 0.5824 | −0.0481 |
| PHE130\|127: | 0.7187 | −0.8269 |
| SER131\|128: | 0.7047 | −0.7468 |
| THR132\|129: | 0.6118 | −0.2161 |
| LEU133\|130: | 0.3388 | 1.3444 |
| LYS134\|131: | 0.4548 | 0.6811 |
| SER135\|132: | 0.7691 | −1.1146 |
| THR136\|133: | 0.5884 | −0.0824 |
| VAL137\|134: | 0.3509 | 1.2753 |
| GLU138\|135: | 0.7401 | −0.9489 |
| ALA139\|136: | 0.7305 | −0.8940 |
| ILE140\|137: | 0.3843 | 1.0843 |
| TRP141\|138: | 0.5456 | 0.1626 |
| ALA142\|139: | 0.7951 | −1.2633 |
| GLY143\|140: | 0.7257 | −0.8667 |
| ILE144\|141: | 0.5792 | −0.0298 |
| LYS145\|142: | 0.5934 | −0.1105 |
| ALA146\|143: | 0.8182 | −1.3952 |
| THR147\|144: | 0.6219 | −0.2734 |
| GLU148\|145: | 0.4550 | 0.6799 |
| ALA149\|146: | 0.8117 | −1.3581 |
| ALA150\|147: | 0.7628 | −1.0789 |
| VAL151\|148: | 0.5229 | 0.2922 |
| SER152\|149: | 0.8837 | −1.7697 |
| GLU153\|150: | 0.7225 | −0.8486 |
| GLU154\|151: | 0.7599 | −1.0620 |
| PHE155\|152: | 0.4945 | 0.4547 |
| GLY156\|153: | 0.8008 | −1.2959 |
| LEU157\|154: | 0.6979 | −0.7079 |
| ALA158\|155: | 0.8341 | −1.4861 |
| PRO159\|156: | 0.8239 | −1.4278 |
| PHE160\|157: | 0.7986 | −1.2835 |
| LEU161\|158: | 0.3388 | 1.3444 |
| PRO162\|159: | 0.3215 | 1.4434 |
| ASP163\|160: | 0.7159 | −0.8109 |
| GLN164\|161: | 0.7316 | −0.9005 |
| ILE165\|162: | 0.4256 | 0.8482 |
| HIS166\|163: | 0.7305 | −0.8944 |
| PHE167\|164: | 0.3433 | 1.3188 |
| VAL168\|165: | 0.5545 | 0.1115 |
| HIS169\|166: | 0.6417 | −0.3870 |
| SER170\|167: | 0.5368 | 0.2130 |
| GLN171\|168: | 0.5373 | 0.2097 |
| GLU172\|169: | 0.4464 | 0.7291 |
| LEU173\|170: | 0.3388 | 1.3444 |
| LEU174\|171: | 0.6675 | −0.5340 |
| SER175\|172: | 0.7617 | −1.0728 |
| ARG176\|173: | 0.6549 | −0.4621 |
| TYR177\|174: | 0.4950 | 0.4516 |
| PRO178\|175: | 0.3215 | 1.4434 |
| ASP179\|176: | 0.6731 | −0.5662 |
| LEU180\|177: | 0.4627 | 0.6360 |
| ASP181\|178: | 0.6812 | −0.6128 |
| ALA182\|179: | 0.6192 | −0.2579 |
| LYS183\|180: | 0.5430 | 0.1772 |
| GLY184\|181: | 0.7081 | −0.7664 |
| ARG185\|182: | 0.3417 | 1.3279 |
| GLU186\|183: | 0.3478 | 1.2929 |
| ARG187\|184: | 0.7572 | −1.0469 |
| ALA188\|185: | 0.5548 | 0.1100 |
| ILE189\|186: | 0.4672 | 0.6102 |
| ALA190\|187: | 0.5685 | 0.0316 |
| LYS191\|188: | 0.4883 | 0.4899 |
| ASP192\|189: | 0.6094 | −0.2019 |
| LEU193\|190: | 0.7459 | −0.9823 |
| GLY194\|191: | 0.5161 | 0.3311 |
| ALA195\|192: | 0.3956 | 1.0198 |
| VAL196\|193: | 0.4584 | 0.6608 |
| PHE197\|194: | 0.3433 | 1.3188 |
| LEU198\|195: | 0.4775 | 0.5515 |
| VAL199\|196: | 0.6844 | −0.6309 |
| GLY200\|197: | 0.6204 | −0.2649 |
| ILE201\|198: | 0.3537 | 1.2588 |
| GLY202\|199: | 0.3172 | 1.4676 |
| GLY203\|200: | 0.6620 | −0.5025 |
| LYS204\|201: | 0.7239 | −0.8565 |
| LEU205\|202: | 0.3388 | 1.3444 |
| SER206\|203: | 0.7312 | −0.8982 |
| ASP207\|204: | 0.5367 | 0.2132 |
| GLY208\|205: | 0.4028 | 0.9787 |
| HIS209\|206: | 0.6750 | −0.5770 |
| ARG210\|207: | 0.6895 | −0.6601 |
| HIS211\|208: | 0.4377 | 0.7790 |
| ASP212\|209: | 0.4180 | 0.8917 |
| VAL213\|210: | 0.6853 | −0.6357 |
| ARG214\|211: | 0.3417 | 1.3279 |
| ALA215\|212: | 0.4287 | 0.8304 |
| PRO216\|213: | 0.4119 | 0.9263 |
| ASP217\|214: | 0.4107 | 0.9336 |
| TYR218\|215: | 0.3396 | 1.3399 |
| ASP219\|216: | 0.3380 | 1.3488 |
| ASP220\|217: | 0.4151 | 0.9084 |
| TRP221\|218: | 0.4065 | 0.9575 |
| SER222\|219: | 0.7261 | −0.8691 |
| THR223\|220: | 0.8496 | −1.5751 |
| PRO224\|221: | 0.8919 | −1.8166 |
| SER225\|222: | 0.8405 | −1.5231 |
| GLU226\|223: | 0.8460 | −1.5542 |
| LEU227\|224: | 0.9100 | −1.9201 |
| GLY228\|225: | 0.8682 | −1.6810 |
| HIS229\|226: | 0.8938 | −1.8276 |
| ALA230\|227: | 0.9263 | −2.0135 |
| GLY231\|228: | 0.7599 | −1.0620 |
| LEU232\|229: | 0.3388 | 1.3444 |
| ASN233\|230: | 0.3530 | 1.2630 |
| GLY234\|231: | 0.3172 | 1.4676 |
| ASP235\|232: | 0.3380 | 1.3488 |
| ILE236\|233: | 0.4827 | 0.5220 |
| LEU237\|234: | 0.4818 | 0.5272 |
| VAL238\|235: | 0.5846 | −0.0607 |
| TRP239\|236: | 0.4952 | 0.4502 |
| ASN240\|237: | 0.6521 | −0.4464 |
| PRO241\|238: | 0.6319 | −0.3308 |
| VAL242\|239: | 0.7452 | −0.9784 |
| LEU243\|240: | 0.5073 | 0.3811 |
| GLU244\|241: | 0.7281 | −0.8804 |
| ASP245\|242: | 0.7918 | −1.2446 |
| ALA246\|243: | 0.5719 | 0.0123 |
| PHE247\|244: | 0.5793 | −0.0300 |
| GLU248\|245: | 0.3478 | 1.2929 |
| LEU249\|246: | 0.5319 | 0.2407 |
| SER250\|247: | 0.3527 | 1.2650 |
| SER251\|248: | 0.3527 | 1.2650 |
| MET252\|249: | 0.3653 | 1.1930 |
| GLY253\|250: | 0.4003 | 0.9927 |
| ILE254\|251: | 0.2450 | 1.8804 |
| ARG255\|252: | 0.1873 | 2.2101 |
| VAL256\|253: | 0.1972 | 2.1537 |
| ASP257\|254: | 0.4263 | 0.8444 |
| ALA258\|255: | 0.5687 | 0.0303 |
| ASP259\|256: | 0.6762 | −0.5842 |
| THR260\|257: | 0.6613 | −0.4989 |
| LEU261\|258: | 0.2881 | 1.6339 |
| LYS262\|259: | 0.7259 | −0.8679 |
| HIS263\|260: | 0.6706 | −0.5518 |
| GLN264\|261: | 0.2071 | 2.0968 |
| LEU265\|262: | 0.4412 | 0.7588 |
| ALA266\|263: | 0.7243 | −0.8587 |
| LEU267\|264: | 0.7291 | −0.8863 |
| THR268\|265: | 0.6420 | −0.3885 |
| GLY269\|266: | 0.6637 | −0.5126 |
| ASP270\|267: | 0.7579 | −1.0507 |
| GLU271\|268: | 0.5332 | 0.2333 |
| ASP272\|269: | 0.5773 | −0.0188 |
| ARG273\|270: | 0.4635 | 0.6314 |
| LEU274\|271: | 0.5859 | −0.0680 |
| GLU275\|272: | 0.6966 | −0.7003 |
| LEU276\|273: | 0.5684 | 0.0320 |
| GLU277\|274: | 0.7766 | −1.1575 |
| TRP278\|275: | 0.5305 | 0.2488 |
| HIS279\|276: | 0.2824 | 1.6665 |
| GLN280\|277: | 0.4868 | 0.4982 |
| ALA281\|278: | 0.7708 | −1.1247 |
| LEU282\|279: | 0.4080 | 0.9490 |
| LEU283\|280: | 0.5131 | 0.3483 |
| ARG284\|281: | 0.5972 | −0.1325 |
| GLY285\|282: | 0.5813 | −0.0418 |
| GLU286\|283: | 0.7598 | −1.0616 |
| MET287\|284: | 0.5211 | 0.3024 |
| PRO288\|285: | 0.4367 | 0.7846 |
| GLN289\|286: | 0.6023 | −0.1614 |
| THR290\|287: | 0.4399 | 0.7666 |
| ILE291\|288: | 0.2899 | 1.6236 |
| GLY292\|289: | 0.2515 | 1.8433 |
| GLY293\|290: | 0.2515 | 1.8433 |
| GLY294\|291: | 0.2515 | 1.8433 |
| ILE295\|292: | 0.2899 | 1.6236 |
| GLY296\|293: | 0.2515 | 1.8433 |
| GLN297\|294: | 0.3356 | 1.3627 |
| SER298\|295: | 0.2893 | 1.6271 |
| ARG299\|296: | 0.2775 | 1.6944 |
| LEU300\|297: | 0.4523 | 0.6958 |
| THR301\|298: | 0.5627 | 0.0645 |
| MET302\|299: | 0.4579 | 0.6634 |
| LEU303\|300: | 0.5095 | 0.3685 |
| LEU304\|301: | 0.4514 | 0.7005 |
| LEU305\|302: | 0.2743 | 1.7126 |
| GLN306\|303: | 0.6888 | −0.6557 |
| LEU307\|304: | 0.4791 | 0.5427 |
| PRO308\|305: | 0.7505 | −1.0083 |
| HIS309\|306: | 0.2824 | 1.6665 |
| ILE310\|307: | 0.3801 | 1.1081 |
| GLY311\|308: | 0.2515 | 1.8433 |
| GLN312\|309: | 0.4121 | 0.9254 |
| VAL313\|310: | 0.3310 | 1.3887 |
| GLN314\|311: | 0.2954 | 1.5923 |
| ALA315\|312: | 0.6228 | −0.2787 |
| GLY316\|313: | 0.4341 | 0.7996 |
| VAL317\|314: | 0.6357 | −0.3524 |
| TRP318\|315: | 0.2596 | 1.7969 |
| PRO319\|316: | 0.5518 | 0.1267 |
| ALA320\|317: | 0.7712 | −1.1267 |
| ALA321\|318: | 0.6837 | −0.6270 |
| VAL322\|319: | 0.6665 | −0.5282 |
| ARG323\|320: | 0.7671 | −1.1032 |
| GLU324\|321: | 0.6500 | −0.4341 |
| SER325\|322: | 0.7396 | −0.9465 |
| VAL326\|323: | 0.7524 | −1.0194 |
| PRO327\|324: | 0.7538 | −1.0277 |
| SER328\|325: | 0.7496 | −1.0033 |
| LEU329\|326: | 0.7697 | −1.1182 |
| LEU330\|327: | 0.6871 | −0.6461 |

Table 3 lists the raw residue conservation scores and corresponding residue conservation z-scores for each residue of the alpha chain of PDB:12asA.

Table 4 lists the surface orientation score for each query residue (—i.e. both chains) under the column headed, “Surface Orient.Score”.

| | Surf. |
|---|---|
| Residue | Orient. |
| Id. | Score |
| ARG10\|7 | 0.5278 |
| ARG10\|334 | 0.5357 |
| ARG100\|97 | 0.7174 |
| ARG100\|424 | 0.7143 |
| PRO101\|98 | 0.5833 |
| PRO101\|425 | 1.0000 |
| ASP102\|99 | 0.4000 |
| ASP102\|426 | 0.4167 |
| GLU103\|100 | 0.3784 |
| GLU103\|427 | 0.4889 |
| ASP104\|101 | 0.0952 |
| ASP104\|428 | 0.3103 |
| ARG105\|102 | 0.0417 |
| ARC105\|429 | 0.2727 |
| LEU106\|103 | 1.0000 |
| LEU106\|430 | 1.0000 |
| SER107\|104 | 0.4242 |
| SER107\|431 | 0.3333 |
| PRO108\|105 | 0.5833 |
| PRO108\|432 | 0.5600 |
| LEU109\|106 | 0.6571 |
| LEU109\|433 | 0.6757 |
| GLN11\|8 | 0.3929 |
| GLN11\|335 | 0.5357 |
| HIS110\|107 | 0.5517 |
| HIS110\|434 | 0.6053 |
| SER111\|108 | 1.0000 |
| SER111\|435 | 1.0000 |
| VAL112\|109 | 1.0000 |
| VAL112\|436 | 1.0000 |
| TYR113\|110 | 0.7451 |
| TYR113\|437 | 0.6731 |
| VAL114\|111 | 0.7297 |
| VAL114\|438 | 0.7143 |
| ASP115\|112 | 0.6279 |
| ASP115\|439 | 0.7660 |
| GLN116\|113 | 0.6061 |
| GLN116\|440 | 0.5862 |
| ASP118\|115 | 1.0000 |
| ASP118\|442 | 1.0000 |
| TRP119\|443 | 0.0000 |
| GLU120\|117 | 0.0000 |
| GLU120\|444 | 0.0000 |
| ARG121\|118 | 0.5455 |
| ARG121\|445 | 0.8125 |
| VAL122\|119 | 0.4545 |
| VAL122\|446 | 0.6471 |
| MET123\|120 | 1.0000 |
| MET123\|447 | 1.0000 |
| GLY124\|121 | 0.2381 |
| GLY124\|448 | 0.2609 |
| ASP125\|122 | 0.0833 |
| ASP125\|449 | 0.0833 |
| GLY126\|123 | 0.1000 |
| GLY126\|450 | 0.1111 |
| GLU127\|124 | 0.2800 |
| GLU127\|451 | 0.2308 |
| ARG128\|125 | 0.5357 |
| ARG128\|452 | 0.5769 |
| GLN129\|126 | 0.4167 |
| GLN129\|453 | 0.4167 |
| SER13\|10 | 0.5429 |
| SER13\|337 | 0.5625 |
| PHE130\|127 | 0.7895 |
| PHE130\|454 | 0.8889 |
| SER131\|128 | 0.2500 |
| SER131\|455 | 0.2500 |
| THR132\|129 | 0.5714 |
| THR132\|456 | 0.5200 |
| LYS134\|131 | 0.8889 |
| LYS134\|458 | 0.4211 |
| SER135\|132 | 0.4000 |
| SER135\|459 | 0.3478 |
| THR136\|133 | 0.7500 |
| THR136\|460 | 0.7273 |
| GLU138\|135 | 0.5385 |
| GLU138\|462 | 0.6000 |
| ALA139\|136 | 0.5000 |
| ALA139\|463 | 0.4483 |
| PHE14\|11 | 0.5000 |
| PHE14\|338 | 0.4583 |
| ILE140\|137 | 1.0000 |
| ILE140\|464 | 1.0000 |
| TRP141\|138 | 1.0000 |
| TRP141\|465 | 1.0000 |
| ALA142\|139 | 0.4828 |
| ALA 142\|466 | 0.5714 |
| GLY143\|140 | 0.3125 |
| GLY143\|467 | 0.5000 |
| ILE144\|141 | 1.0000 |
| ILE144\|468 | 1.0000 |
| LYS145\|142 | 0.4516 |
| LYS145\|469 | 0.5556 |
| ALA146\|143 | 0.4074 |
| ALA146\|470 | 0.3448 |
| GLU148\|145 | 1.0000 |
| GLU148\|472 | 1.0000 |
| ALA149\|146 | 0.2917 |
| ALA149\|473 | 0.3478 |
| ALA150\|147 | 0.5000 |
| ALA150\|474 | 0.5000 |
| VAL151\|148 | 1.0000 |
| VAL151\|475 | 1.0000 |
| SER152\|149 | 0.4000 |
| SER152\|476 | 0.4000 |
| GLU153\|150 | 0.1333 |
| GLU153\|477 | 0.0667 |
| GLU154\|151 | 0.1765 |
| GLU154\|478 | 0.1765 |
| PHE155\|152 | 0.3000 |
| PHE155\|479 | 0.2963 |
| GLY156\|153 | 0.0500 |
| GLY156\|480 | 0.0500 |
| LEU157\|154 | 0.3793 |
| LEU157\|481 | 0.3793 |
| ALA158\|155 | 0.1481 |
| ALA158\|482 | 0.1852 |
| PRO159\|156 | 0.3030 |
| PRO159\|483 | 0.3077 |
| LYS16\|13 | 0.7955 |
| LYS16\|340 | 0.7568 |
| PHE160\|157 | 0.2857 |
| PHE160\|484 | 0.3871 |
| LEU161\|158 | 1.0000 |
| LEU161\|485 | 1.0000 |
| PRO162\|159 | 0.2188 |
| PRO162\|486 | 0.2800 |
| ASP163\|160 | 0.0000 |
| ASP163\|487 | 0.0556 |
| GLN164\|161 | 0.1053 |
| GLN164\|488 | 0.2000 |
| ILE165\|162 | 1.0000 |
| ILE165\|489 | 1.0000 |
| HIS166\|163 | 0.5000 |
| HIS166\|490 | 0.5909 |
| PHE167\|164 | 0.8696 |
| PHE167\|491 | 0.7692 |
| VAL168\|165 | 1.0000 |
| VAL168\|492 | 0.9000 |
| HIS169\|166 | 1.0000 |
| HIS169\|493 | 1.0000 |
| SER17\|14 | 0.4615 |
| SER17\|341 | 0.4583 |
| GLN171\|168 | 0.6250 |
| GLN171\|495 | 0.6897 |
| GLU172\|169 | 0.6538 |
| GLU172\|496 | 0.6538 |
| LEU174\|171 | 0.5652 |
| LEU174\|498 | 0.5652 |
| SER175\|172 | 0.1500 |
| SER175\|499 | 0.0952 |
| ARG176\|173 | 0.1818 |
| ARG176\|500 | 0.2000 |
| TYR177\|174 | 0.2778 |
| TYR177\|501 | 0.2727 |
| PRO178\|175 | 0.1667 |
| PRO178\|502 | 0.0500 |
| ASP179\|176 | 0.0000 |
| ASP179\|503 | 0.0000 |
| HIS18\|15 | 0.5652 |
| HIS18\|342 | 0.5652 |
| LEU180\|177 | 0.1304 |
| LEU180\|504 | 0.1739 |
| ASP181\|178 | 0.2273 |
| ASP181\|505 | 0.3158 |
| ALA182\|179 | 0.6400 |
| ALA182\|506 | 0.6071 |
| LYS183\|180 | 0.3846 |
| LYS183\|507 | 0.4545 |
| GLY184\|181 | 0.3810 |
| GLY184\|508 | 0.3500 |
| ARG185\|182 | 1.0000 |
| ARC185\|509 | 0.6552 |
| GLU186\|183 | 0.7931 |
| GLU186\|510 | 0.7188 |
| ARG187\|184 | 0.5000 |
| ARG187\|511 | 0.4688 |
| ALA188\|185 | 0.5000 |
| ALA188\|512 | 0.4091 |
| ILE189\|186 | 1.0000 |
| ILE189\|513 | 0.0000 |
| PHE19\|16 | 0.0000 |
| PHE19\|343 | 0.0000 |
| ALA190\|187 | 0.0000 |
| LYS191\|188 | 0.3462 |
| LYS191\|515 | 0.3478 |
| ASP192\|189 | 0.1765 |
| ASP192\|516 | 0.2353 |
| LEU193\|190 | 0.4231 |
| LEU193\|517 | 0.5000 |
| GLY194\|191 | 0.4400 |
| GLY194\|518 | 0.5714 |
| ALA195\|192 | 0.0000 |
| VAL196\|193 | 0.0000 |
| LEU198\|522 | 1.0000 |
| SER20\|17 | 0.5185 |
| SER20\|344 | 0.5556 |
| GLY200\|197 | 0.6000 |
| GLY200\|524 | 0.0000 |
| ILE201\|198 | 1.0000 |
| ILE201\|525 | 1.0000 |
| GLY202\|199 | 0.0000 |
| GLY202\|526 | 0.0000 |
| GLY203\|200 | 0.5000 |
| GLY203\|527 | 0.6250 |
| LYS204\|201 | 0.1500 |
| LYS204\|528 | 0.3750 |
| LEU205\|202 | 0.4000 |
| LEU205\|529 | 1.0000 |
| SER206\|203 | 0.1667 |
| SER206\|530 | 0.1053 |
| ASP207\|204 | 0.0526 |
| ASP207\|531 | 0.0870 |
| GLY208\|205 | 0.0667 |
| GLY208\|532 | 0.1053 |
| HIS209\|206 | 0.1000 |
| HIS209\|533 | 0.1111 |
| ARG21\|18 | 0.3478 |
| ARG21\|345 | 0.4091 |
| ARG210\|207 | 0.5357 |
| ARG210\|534 | 0.3929 |
| HIS211\|208 | 1.0000 |
| HIS211\|535 | 1.0000 |
| ASP212\|209 | 0.6897 |
| ASP212\|536 | 0.5417 |
| VAL213\|210 | 0.4516 |
| VAL213\|537 | 0.3667 |
| ARG214\|211 | 0.6364 |
| ARG214\|538 | 0.5870 |
| ALA215\|212 | 1.0000 |
| ALA215\|539 | 1.0000 |
| PRO216\|213 | 1.0000 |
| PRO216\|540 | 1.0000 |
| ASP217\|214 | 0.0000 |
| TYR218\|215 | 0.7660 |
| TYR218\|542 | 0.6977 |
| ASP219\|216 | 1.0000 |
| ASP219\|543 | 1.0000 |
| GLN22\|19 | 0.5238 |
| GLN22\|346 | 0.5000 |
| TRP221\|218 | 1.0000 |
| TRP221\|545 | 1.0000 |
| SER222\|219 | 0.6400 |
| SER222\|546 | 0.5769 |
| THR223\|220 | 0.5909 |
| THR223\|547 | 0.4583 |
| PRO224\|221 | 0.2273 |
| PRO224\|548 | 0.3182 |
| SER225\|222 | 1.0000 |
| SER225\|549 | 0.5294 |
| GLU226\|223 | 0.1500 |
| GLU226\|550 | 0.1579 |
| LEU227\|224 | 0.3182 |
| LEU227\|551 | 0.2381 |
| GLY228\|225 | 0.0588 |
| GLY228\|552 | 0.0526 |
| HIS229\|226 | 0.3913 |
| HIS229\|553 | 0.4783 |
| LEU23\|20 | 1.0000 |
| LEU23\|347 | 1.0000 |
| ALA230\|227 | 0.5217 |
| ALA230\|554 | 0.5417 |
| LEU232\|229 | 1.0000 |
| LEU232\|556 | 1.0000 |
| ASP235\|232 | 1.0000 |
| ASP235\|559 | 0.9211 |
| ILE236\|233 | 1.0000 |
| ILE236\|560 | 1.0000 |
| LEU237\|234 | 1.0000 |
| LEU237\|561 | 1.0000 |
| VAL238\|235 | 1.0000 |
| TRP239\|236 | 0.6571 |
| TRP239\|563 | 0.5769 |
| GLU24\|21 | 0.5385 |
| GLU24\|348 | 0.5862 |
| ASN240\|237 | 1.0000 |
| ASN240\|564 | 1.0000 |
| PRO241\|238 | 0.4000 |
| PRO241\|565 | 0.2500 |
| VAL242\|239 | 0.3103 |
| VAL242\|566 | 0.2308 |
| LEU243\|240 | 0.2963 |
| LEU243\|567 | 0.2857 |
| GLU244\|241 | 0.0833 |
| GLU244\|568 | 0.2609 |
| ASP245\|242 | 0.5714 |
| ASP245\|569 | 0.5938 |
| ALA246\|243 | 0.7714 |
| ALA246\|570 | 1.0000 |
| PHE247\|244 | 1.0000 |
| PHE247\|571 | 1.0000 |
| GLU248\|245 | 0.5957 |
| GLU248\|572 | 0.6429 |
| LEU249\|246 | 1.0000 |
| LEU249\|573 | 1.0000 |
| GLU25\|22 | 0.1111 |
| GLU25\|349 | 0.1538 |
| SER250\|247 | 1.0000 |
| SER250\|574 | 1.0000 |
| SER251\|248 | 0.6905 |
| SER251\|575 | 0.6591 |
| MET252\|249 | 0.0000 |
| MET252\|576 | 1.0000 |
| ARG255\|252 | 1.0000 |
| ARG255\|579 | 1.0000 |
| VAL256\|253 | 0.0000 |
| ASP257\|254 | 0.5333 |
| ASP257\|581 | 0.6000 |
| ALA258\|255 | 0.4211 |
| ALA258\|582 | 0.4737 |
| ASP259\|256 | 0.1111 |
| ASP259\|583 | 0.0588 |
| ARG26\|23 | 0.1429 |
| ARG26\|350 | 0.2222 |
| THR260\|257 | 0.6957 |
| THR260\|584 | 0.5263 |
| LYS262\|259 | 0.6000 |
| LYS262\|586 | 0.5263 |
| HIS263\|260 | 0.3000 |
| HIS263\|587 | 0.3333 |
| LEU265\|262 | 0.0000 |
| LEU265\|589 | 1.0000 |
| ALA266\|263 | 0.1111 |
| ALA266\|590 | 0.2105 |
| LEU267\|264 | 0.1429 |
| LEU267\|591 | 0.2759 |
| THR268\|265 | 0.3333 |
| THR268\|592 | 0.3235 |
| GLY269\|266 | 0.0500 |
| GLY269\|593 | 0.0870 |
| LEU27\|24 | 0.6000 |
| LEU27\|351 | 1.0000 |
| ASP270\|267 | 0.5455 |
| ASP270\|594 | 0.4400 |
| GLU271\|268 | 0.3684 |
| GLU271\|595 | 0.4118 |
| ASP272\|269 | 0.0870 |
| ASP272\|596 | 0.0870 |
| ARG273\|270 | 0.6296 |
| ARG273\|597 | 0.6129 |
| LEU274\|271 | 0.5000 |
| LEU274\|598 | 0.3158 |
| GLU275\|272 | 0.1667 |
| GLU275\|599 | 0.2273 |
| LEU276\|273 | 0.4138 |
| LEU276\|600 | 0.3462 |
| GLU277\|274 | 0.3611 |
| GLU277\|601 | 0.3000 |
| TRP278\|275 | 1.0000 |
| TRP278\|602 | 1.0000 |
| HIS279\|276 | 1.0000 |
| HIS279\|603 | 1.0000 |
| GLY28\|25 | 0.3929 |
| GLY28\|352 | 0.3824 |
| GLN280\|277 | 0.4286 |
| GLN280\|604 | 0.3500 |
| ALA281\|278 | 0.3600 |
| ALA281\|605 | 0.5000 |
| LEU282\|279 | 1.0000 |
| LEU282\|606 | 1.0000 |
| LEU283\|280 | 0.5000 |
| LEU283\|607 | 0.5500 |
| ARG284\|281 | 0.0000 |
| ARG284\|608 | 0.0000 |
| GLY285\|282 | 0.0526 |
| GLY285\|609 | 0.1176 |
| GLU286\|283 | 0.2727 |
| GLU286\|610 | 0.2963 |
| MET287\|284 | 1.0000 |
| MET287\|611 | 1.0000 |
| PRO288\|285 | 0.4286 |
| PRO288\|612 | 0.4167 |
| GLN289\|286 | 0.5625 |
| GLN289\|613 | 0.5429 |
| LEU29\|26 | 0.0000 |
| LEU29\|353 | 0.0000 |
| ILE291\|288 | 1.0000 |
| ILE291\|615 | 1.0000 |
| GLY293\|290 | 0.6190 |
| GLY293\|617 | 0.7000 |
| GLY294\|291 | 0.7692 |
| GLY294\|618 | 0.7200 |
| ILE295\|292 | 1.0000 |
| ILE295\|619 | 1.0000 |
| GLY296\|293 | 0.7931 |
| GLY296\|620 | 0.7667 |
| GLN297\|294 | 1.0000 |
| GLN297\|621 | 0.8667 |
| SER298\|295 | 1.0000 |
| SER298\|622 | 1.0000 |
| ARG299\|296 | 0.5250 |
| ARG299\|623 | 0.7027 |
| ILE30\|27 | 0.4839 |
| ILE30\|354 | 0.6429 |
| LEU300\|297 | 1.0000 |
| THR301\|298 | 0.0000 |
| MET302\|299 | 0.0000 |
| MET302\|626 | 0.0000 |
| LEU303\|300 | 1.0000 |
| LEU303\|627 | 0.0000 |
| LEU304\|301 | 0.0000 |
| LEU304\|628 | 0.0000 |
| LEU305\|302 | 0.0000 |
| LEU305\|629 | 0.0000 |
| GLN306\|303 | 0.4722 |
| GLN306\|630 | 0.4688 |
| LEU307\|304 | 0.6667 |
| LEU307\|631 | 0.6333 |
| PRO308\|305 | 0.5000 |
| PR0308\|632 | 0.5152 |
| HIS309\|306 | 0.7586 |
| HIS309\|633 | 1.0000 |
| GLU31\|28 | 0.6176 |
| GLU31\|355 | 0.6765 |
| ILE310\|307 | 1.0000 |
| ILE310\|634 | 1.0000 |
| GLN312\|309 | 0.5385 |
| GLN312\|636 | 0.4800 |
| VAL313\|310 | 1.0000 |
| VAL313\|637 | 0.0000 |
| GLN314\|311 | 1.0000 |
| GLN314\|638 | 1.0000 |
| ALA315\|312 | 1.0000 |
| ALA315\|639 | 1.0000 |
| GLY316\|313 | 0.5385 |
| GLY316\|640 | 0.6304 |
| VAL317\|314 | 0.8276 |
| VAL317\|641 | 0.6250 |
| TRP318\|315 | 1.0000 |
| TRP318\|642 | 1.0000 |
| PRO319\|316 | 0.4074 |
| PRO319\|643 | 0.3667 |
| VAL32\|29 | 0.0000 |
| VAL32\|356 | 0.0000 |
| ALA320\|317 | 0.3438 |
| ALA320\|644 | 0.1429 |
| ALA321\|318 | 0.1333 |
| ALA321\|645 | 0.2143 |
| VAL322\|319 | 0.2857 |
| VAL322\|646 | 0.3077 |
| ARG323\|320 | 0.5135 |
| ARG323\|647 | 0.4750 |
| GLU324\|321 | 0.1053 |
| GLU324\|648 | 0.0556 |
| SER325\|322 | 0.0625 |
| SER325\|649 | 0.0625 |
| VAL326\|323 | 0.3103 |
| VAL326\|650 | 0.2857 |
| PRO327\|324 | 0.1875 |
| PRO327\|651 | 0.1364 |
| SER328\|325 | 0.5161 |
| SER328\|652 | 0.3810 |
| LEU329\|326 | 0.5000 |
| LEU329\|653 | 0.5357 |
| GLN33\|30 | 0.6400 |
| GLN33\|357 | 0.6304 |
| LEU330\|327 | 0.6538 |
| LEU330\|654 | 0.4808 |
| ALA34\|31 | 0.6000 |
| ALA34\|358 | 0.5882 |
| PRO35\|32 | 0.6977 |
| PRO35\|359 | 0.6444 |
| ILE36\|33 | 1.0000 |
| ILE36\|360 | 1.0000 |
| LEU37\|34 | 1.0000 |
| LEU37\|361 | 1.0000 |
| SER38\|35 | 0.5641 |
| SER38\|362 | 0.6571 |
| ARG39\|36 | 0.6087 |
| ARG39\|363 | 0.6154 |
| ALA4\|1 | 0.1429 |
| ALA4\|328 | 0.1111 |
| VAL40\|37 | 0.3438 |
| VAL40\|364 | 0.4074 |
| GLY41\|38 | 0.2813 |
| GLY41\|365 | 0.3333 |
| ASP42\|39 | 0.4872 |
| ASP42\|366 | 0.4706 |
| GLY43\|40 | 0.6667 |
| GLY43\|367 | 0.3750 |
| THR44\|41 | 0.3929 |
| THR44\|368 | 0.4211 |
| GLN45\|42 | 0.7692 |
| GLN45\|369 | 0.7692 |
| ASP46\|43 | 1.0000 |
| ASP46\|370 | 1.0000 |
| ASN47\|44 | 0.6970 |
| ASN47\|371 | 0.5806 |
| LEU48\|45 | 0.5000 |
| LEU48\|372 | 0.5238 |
| SER49\|46 | 0.3871 |
| SER49\|373 | 0.3714 |
| TYR5\|2 | 0.2973 |
| TYR5\|329 | 0.2683 |
| GLY50\|47 | 0.4074 |
| GLY50\|374 | 0.4400 |
| ALA51\|48 | 0.1667 |
| ALA51\|375 | 0.2500 |
| GLU52\|49 | 0.3333 |
| GLU52\|376 | 0.3243 |
| LYS53\|50 | 0.4375 |
| LYS53\|377 | 0.4063 |
| ALA54\|51 | 1.0000 |
| ALA54\|378 | 1.0000 |
| VAL55\|52 | 1.0000 |
| VAL55\|379 | 0.3704 |
| GLN56\|53 | 0.5000 |
| GLN56\|380 | 0.6111 |
| VAL57\|54 | 1.0000 |
| VAL57\|381 | 1.0000 |
| LYS58\|55 | 0.4063 |
| LYS58\|382 | 0.4333 |
| VAL59\|56 | 0.0000 |
| VAL59\|383 | 1.0000 |
| ILE6\|3 | 0.4857 |
| ILE6\|330 | 0.4828 |
| LYS60\|57 | 0.3333 |
| LYS60\|384 | 0.3043 |
| ALA61\|58 | 0.5000 |
| ALA61\|385 | 0.3913 |
| LEU62\|59 | 0.3333 |
| LEU62\|386 | 0.3824 |
| PRO63\|60 | 0.0526 |
| PRO63\|387 | 0.0526 |
| ASP64\|61 | 0.0000 |
| ASP64\|388 | 0.0000 |
| ALA65\|62 | 0.2917 |
| ALA65\|389 | 0.2581 |
| GLN66\|63 | 0.2759 |
| GLN66\|390 | 0.2727 |
| PHE67\|64 | 1.0000 |
| PHE67\|391 | 1.0000 |
| GLU68\|65 | 0.6316 |
| GLU68\|392 | 0.4595 |
| VAL69\|66 | 1.0000 |
| VAL69\|393 | 1.0000 |
| ALA7\|4 | 0.4444 |
| ALA7\|331 | 0.4545 |
| VAL70\|67 | 1.0000 |
| VAL70\|394 | 1.0000 |
| HIS71\|68 | 1.0000 |
| HIS71\|395 | 1.0000 |
| SER72\|69 | 0.5185 |
| SER72\|396 | 0.5000 |
| LEU73\|70 | 1.0000 |
| LEU73\|397 | 1.0000 |
| ALA74\|71 | 0.6176 |
| ALA74\|398 | 0.6774 |
| LYS75\|72 | 1.0000 |
| LYS75\|399 | 1.0000 |
| TRP76\|73 | 0.9048 |
| TRP76\|400 | 0.7619 |
| LYS77\|74 | 1.0000 |
| LYS77\|401 | 1.0000 |
| ARG78\|75 | 1.0000 |
| ARG78\|402 | 1.0000 |
| GLN79\|76 | 0.6486 |
| GLN79\|403 | 0.5882 |
| LYS8\|5 | 0.5484 |
| LYS8\|332 | 0.6774 |
| THR80\|77 | 0.5652 |
| THR80\|404 | 0.7200 |
| LEU81\|405 | 0.0000 |
| GLY82\|79 | 0.5833 |
| GLY82\|406 | 0.4583 |
| GLN83\|80 | 0.4800 |
| GLN83\|407 | 0.2727 |
| HIS84\|81 | 0.3750 |
| HIS84\|408 | 0.5294 |
| ASP85\|82 | 0.1818 |
| ASP85\|409 | 0.3448 |
| PHE86\|83 | 0.3939 |
| PHE86\|410 | 1.0000 |
| SER87\|84 | 0.2105 |
| SER87\|411 | 0.0909 |
| ALA88\|85 | 0.5000 |
| ALA88\|412 | 0.3810 |
| GLY89\|86 | 0.4815 |
| GLY89\|413 | 0.3571 |
| GLN9\|6 | 0.0000 |
| GLN9\|333 | 1.0000 |
| GLU90\|87 | 0.5455 |
| GLU90\|414 | 0.5862 |
| GLY91\|88 | 0.0000 |
| GLY91\|415 | 0.0000 |
| LEU92\|89 | 0.0000 |
| TYR93\|90 | 1.0000 |
| TYR93\|417 | 1.0000 |
| THR94\|91 | 1.0000 |
| THR94\|418 | 0.0000 |
| HIS95\|92 | 0.6341 |
| HIS95\|419 | 0.7949 |
| MET96\|93 | 1.0000 |
| MET96\|420 | 1.0000 |
| LYS97\|94 | 0.7966 |
| LYS97\|421 | 0.8167 |
| LEU99\|96 | 1.0000 |
| LEU99\|423 | 1.0000 |

Table 4 lists the surface orientation score of each surface residue on PDB:12asA.

Table 5 lists the nine largest putative functional clusters among the 63 putative functional clusters identified on the surface of PDB:12asA. Each putative functional cluster is identified by the residues that comprise it, listed under the heading “Residue Id.”, their corresponding residue conservation z-scores, listed under the heading “Residue Cons. Z-score”, and their corresponding surface orientation scores, listed under the heading “Surf. Orient. Score”.

| | Residue Cons. | Surface |
|---|---|---|
| Residue Id. | Z-score | Orient. Score |
| Putative Functional Cluster 1 | | |
| TYR113\|437 | 1.2035 | 0.6731 |
| GLU277\|274 | −0.2344 | 0.3611 |
| TRP278\|275 | 0.8390 | 1.0000 |
| ALA281\|278 | −0.1846 | 0.3600 |
| GLU286\|283 | −0.3789 | 0.2727 |
| MET287\|284 | 0.4933 | 1.0000 |
| PRO288\|285 | 0.2402 | 0.4286 |
| ALA315\|639 | 0.5597 | 1.0000 |
| GLY316\|640 | 0.1839 | 0.6304 |
| VAL317\|641 | −0.5239 | 0.6250 |
| TRP318\|642 | −0.6812 | 1.0000 |
| ALA320\|644 | −0.8467 | 0.1429 |
| ARG323\|647 | −0.7672 | 0.4750 |
| GLU324\|648 | −0.6392 | 0.0556 |
| VAL326\|650 | −0.6913 | 0.2857 |
| SER328\|652 | −0.7267 | 0.3810 |
| LEU329\|653 | −0.6035 | 0.5357 |
| LEU330\|654 | −0.0576 | 0.4808 |
| PRO35\|32 | 0.6308 | 0.6977 |
| ILE36\|33 | 0.9785 | 1.0000 |
| LEU37\|34 | 0.5015 | 1.0000 |
| SER38\|35 | −0.2640 | 0.5641 |
| ARG39\|36 | −0.8028 | 0.6087 |
| VAL40\|37 | −0.8241 | 0.3438 |
| ASP42\|39 | −0.5838 | 0.4872 |
| THR44\|41 | −0.5255 | 0.3929 |
| TYR5\|329 | −0.2105 | 0.2683 |
| LEU62\|59 | −1.0609 | 0.3333 |
| ALA65\|62 | −1.2435 | 0.2917 |
| VAL70\|67 | 0.3724 | 1.0000 |
| LEU73\|70 | 0.1597 | 1.0000 |
| TRP76\|73 | −0.0716 | 0.9048 |
| GLN79\|76 | −0.2690 | 0.6486 |
| THR80\|77 | −1.0156 | 0.5652 |
| GLY82\|79 | −0.6303 | 0.5833 |
| GLN83\|80 | −0.9890 | 0.4800 |
| HIS84\|81 | −1.1824 | 0.3750 |
| ASP85\|82 | −0.9296 | 0.1818 |
| Putative Functional Cluster 2 | | |
| TYR113\|110 | 1.3591 | 0.7451 |
| TYR113\|437 | 1.2035 | 0.6731 |
| ASP115\|112 | 1.4782 | 0.6279 |
| ASP115\|439 | 1.1860 | 0.7660 |
| SER13\|10 | −0.4475 | 0.5429 |
| SER13\|337 | −0.5015 | 0.5625 |
| LYS16\|13 | 0.4705 | 0.7955 |
| LYS16\|340 | 0.4279 | 0.7568 |
| SER17\|341 | −0.8309 | 0.4583 |
| SER20\|344 | −0.4164 | 0.5556 |
| GLU24\|348 | −1.2310 | 0.5862 |
| GLN297\|294 | 0.6433 | 1.0000 |
| GLN297\|621 | 1.0823 | 0.8667 |
| SER298\|295 | 1.4984 | 1.0000 |
| SER298\|622 | 1.5430 | 1.0000 |
| GLU31\|355 | −0.4473 | 0.6765 |
| VAL313\|310 | 0.8007 | 1.0000 |
| VAL313\|637 | 0.8576 | 0.0000 |
| GLN314\|311 | 1.2009 | 1.0000 |
| GLN314\|638 | 1.2677 | 1.0000 |
| ALA315\|312 | 0.4810 | 1.0000 |
| VAL32\|356 | 0.2932 | 0.0000 |
| GLN33\|30 | 0.1648 | 0.6400 |
| GLN33\|357 | 0.0467 | 0.6304 |
| ALA34\|31 | 1.0127 | 0.6000 |
| ALA34\|358 | 0.8826 | 0.5882 |
| PRO35\|32 | 0.6308 | 0.6977 |
| PRO35\|359 | 0.5221 | 0.6444 |
| ILE36\|33 | 0.9785 | 1.0000 |
| ILE36\|360 | 0.8911 | 1.0000 |
| TYR93\|417 | −0.5632 | 1.0000 |
| HIS95\|92 | 0.4904 | 0.6341 |
| HIS95\|419 | 0.4292 | 0.7949 |
| MET96\|420 | 1.0533 | 1.0000 |
| LYS97\|94 | 0.8735 | 0.7966 |
| LYS97\|421 | 1.0996 | 0.8167 |
| Putative Functional Cluster 3 | | |
| PHE130\|454 | −1.0960 | 0.8889 |
| SER131\|455 | −0.6447 | 0.2500 |
| LYS134\|458 | −0.5469 | 0.4211 |
| SER135\|459 | −0.2326 | 0.3478 |
| GLU138\|462 | −0.7060 | 0.6000 |
| ALA139\|463 | −0.0059 | 0.4483 |
| TRP141\|465 | −0.4894 | 1.0000 |
| ALA142\|466 | −0.8590 | 0.5714 |
| LYS145\|469 | −0.9647 | 0.5556 |
| ALA146\|470 | −1.3722 | 0.3448 |
| GLU148\|472 | −0.5588 | 1.0000 |
| ALA149\|473 | −1.5846 | 0.3478 |
| PRO159\|483 | −0.6758 | 0.3077 |
| PHE160\|484 | −0.5698 | 0.3871 |
| LEU161\|485 | −0.7135 | 1.0000 |
| PRO162\|486 | 0.2255 | 0.2800 |
| ASP163\|487 | −0.6398 | 0.0556 |
| GLN164\|488 | −0.1903 | 0.2000 |
| ILE165\|489 | −0.3884 | 1.0000 |
| HIS166\|490 | 0.0030 | 0.5909 |
| PHE167\|491 | −0.4717 | 0.7692 |
| VAL168\|492 | 0.1925 | 0.9000 |
| GLN171\|495 | −0.2180 | 0.6897 |
| GLU172\|496 | −0.6685 | 0.6538 |
| SER175\|499 | −0.0613 | 0.0952 |
| ARG176\|500 | 0.0473 | 0.2000 |
| LYS191\|515 | 0.1421 | 0.3478 |
| ASP192\|516 | 0.2708 | 0.2353 |
| LEU193\|517 | 0.2522 | 0.5000 |
| GLY194\|518 | −0.2075 | 0.5714 |
| GLU226\|550 | −1.3015 | 0.1579 |
| LEU227\|551 | −1.4053 | 0.2381 |
| HIS229\|553 | −1.2991 | 0.4783 |
| TRP239\|563 | −0.1956 | 0.5769 |
| PRO241\|565 | −0.0821 | 0.2500 |
| ARG26\|350 | −0.5651 | 0.2222 |
| Putative Functional Cluster 4 | | |
| VAL122\|446 | −0.2916 | 0.6471 |
| MET123\|447 | −0.2393 | 1.0000 |
| GLY124\|448 | −0.3525 | 0.2609 |
| GLY126\|450 | −0.8894 | 0.1111 |
| ARG128\|452 | −0.5517 | 0.5769 |
| GLN129\|453 | −0.8289 | 0.4167 |
| SER222\|546 | −0.7506 | 0.5769 |
| THR223\|547 | −0.9856 | 0.4583 |
| PRO224\|548 | −1.2497 | 0.3182 |
| SER225\|549 | −1.9512 | 0.5294 |
| GLU226\|550 | −1.3015 | 0.1579 |
| LEU232\|556 | −1.2598 | 1.0000 |
| ASP257\|581 | 0.4583 | 0.6000 |
| ALA258\|582 | 0.1708 | 0.4737 |
| ASP259\|583 | −0.1904 | 0.0588 |
| THR260\|584 | −0.4812 | 0.5263 |
| LYS262\|586 | −0.2567 | 0.5263 |
| HIS263\|587 | −0.8587 | 0.3333 |
| LEU265\|589 | −0.1968 | 1.0000 |
| ALA266\|590 | −0.6345 | 0.2105 |
| LEU274\|598 | −0.2632 | 0.3158 |
| GLN280\|604 | −0.3771 | 0.3500 |
| LEU282\|606 | 0.7223 | 1.0000 |
| LEU283\|607 | −0.0470 | 0.5500 |
| ARG284\|608 | −0.5554 | 0.0000 |
| GLY285\|609 | −0.1093 | 0.1176 |
| GLU286\|610 | −0.3971 | 0.2963 |
| MET287\|611 | 0.4340 | 1.0000 |
| PRO288\|612 | 0.0488 | 0.4167 |
| GLN289\|613 | 0.2905 | 0.5429 |
| ARG78\|402 | 0.9353 | 1.0000 |
| LEU81\|405 | −0.4314 | 0.0000 |
| GLY82\|406 | −0.6392 | 0.4583 |
| ASP85\|409 | −1.1864 | 0.3448 |
| PHE86\|410 | −0.9412 | 1.0000 |
| SER87\|411 | −1.0703 | 0.0909 |
| ALA88\|412 | 0.6256 | 0.3810 |
| Putative Functional Cluster 5 | | |
| VAL122\|119 | −0.7068 | 0.4545 |
| MET123\|120 | −0.4736 | 1.0000 |
| GLY124\|121 | −0.4961 | 0.2381 |
| GLY126\|123 | −0.8402 | 0.1000 |
| ARG128\|125 | −0.5717 | 0.5357 |
| GLN129\|126 | −1.0412 | 0.4167 |
| SER222\|219 | −0.9567 | 0.6400 |
| THR223\|220 | −1.3405 | 0.5909 |
| PRO224\|221 | −1.4313 | 0.2273 |
| SER225\|222 | −2.1340 | 1.0000 |
| LEU232\|229 | −1.3145 | 1.0000 |
| ASP257\|254 | 0.8067 | 0.5333 |
| ALA258\|255 | 0.3680 | 0.4211 |
| ASP259\|256 | −0.1454 | 0.1111 |
| THR260\|257 | −0.5692 | 0.6957 |
| LYS262\|259 | −0.2391 | 0.6000 |
| HIS263\|260 | −0.6794 | 0.3000 |
| GLU271\|268 | −0.3769 | 0.3684 |
| LEU274\|271 | −0.1561 | 0.5000 |
| GLU275\|272 | −0.4234 | 0.1667 |
| GLN280\|277 | −0.4442 | 0.4286 |
| LEU282\|279 | 0.8332 | 1.0000 |
| LEU283\|280 | −0.0470 | 0.5000 |
| ARG284\|281 | −0.6225 | 0.0000 |
| GLY285\|282 | −0.2016 | 0.0526 |
| GLU286\|283 | −0.3789 | 0.2727 |
| MET287\|284 | 0.4933 | 1.0000 |
| PRO288\|285 | 0.2402 | 0.4286 |
| GLN289\|286 | 0.4797 | 0.5625 |
| ARG78\|75 | 1.1560 | 1.0000 |
| PHE86\|83 | −0.9046 | 0.3939 |
| SER87\|84 | −1.2902 | 0.2105 |
| ALA88\|85 | −0.6607 | 0.5000 |
| Putative Functional Cluster 6 | | |
| ARG100\|424 | 1.2179 | 0.7143 |
| SER111\|435 | 1.0295 | 1.0000 |
| VAL114\|438 | 1.5666 | 0.7143 |
| ASP115\|439 | 1.1860 | 0.7660 |
| GLN116\|440 | 2.6940 | 0.5862 |
| ASP118\|442 | 2.8645 | 1.0000 |
| GLU186\|510 | 0.8919 | 0.7188 |
| LEU198\|522 | 1.5269 | 1.0000 |
| ILE201\|525 | 1.8921 | 1.0000 |
| HIS211\|535 | 0.8670 | 1.0000 |
| ARG214\|538 | 1.3341 | 0.5870 |
| ALA215\|539 | 1.6168 | 1.0000 |
| TYR218\|542 | 1.5988 | 0.6977 |
| ASP219\|543 | 2.3869 | 1.0000 |
| ASP235\|559 | 1.8816 | 0.9211 |
| LEU237\|561 | 1.2497 | 1.0000 |
| ALA246\|570 | 0.8437 | 1.0000 |
| GLU248\|572 | 1.7245 | 0.6429 |
| LEU249\|573 | 2.3089 | 1.0000 |
| SER250\|574 | 1.9222 | 1.0000 |
| SER251\|575 | 2.2703 | 0.6591 |
| ARG255\|579 | 2.5792 | 1.0000 |
| GLY293\|617 | 3.0107 | 0.7000 |
| GLY294\|618 | 2.9080 | 0.7200 |
| GLY296\|620 | 2.4335 | 0.7667 |
| ARG299\|623 | 1.7470 | 0.7027 |
| ILE310\|634 | 1.2665 | 1.0000 |
| GLN314\|638 | 1.2677 | 1.0000 |
| ASP46\|370 | 2.1090 | 1.0000 |
| LYS77\|401 | 2.8404 | 1.0000 |
| Putative Functional Cluster 7 | | |
| PHE130\|127 | −1.0345 | 0.7895 |
| SER131\|128 | −1.0627 | 0.2500 |
| LYS134\|131 | −0.5309 | 0.8889 |
| SER135\|132 | −0.5472 | 0.4000 |
| GLU138\|135 | −0.5337 | 0.5385 |
| ALA139\|136 | −0.5873 | 0.5000 |
| TRP141\|138 | 0.3985 | 1.0000 |
| ALA142\|139 | −0.7085 | 0.4828 |
| LYS145\|142 | −0.8566 | 0.4516 |
| ALA146\|143 | −1.3680 | 0.4074 |
| GLU148\|145 | −0.4517 | 1.0000 |
| ALA149\|146 | −1.5722 | 0.2917 |
| PRO159\|156 | −0.5869 | 0.3030 |
| PHE160\|157 | −0.4830 | 0.2857 |
| LEU161\|158 | −0.1432 | 1.0000 |
| PRO162\|159 | 0.1760 | 0.2188 |
| ASP163\|160 | −0.4524 | 0.0000 |
| GLN164\|161 | −0.0558 | 0.1053 |
| ILE165\|162 | −0.1321 | 1.0000 |
| HIS166\|163 | 0.0029 | 0.5000 |
| PHE167\|164 | −0.7776 | 0.8696 |
| LEU193\|190 | 0.3212 | 0.4231 |
| GLY194\|191 | 0.3451 | 0.4400 |
| LEU227\|224 | −1.2101 | 0.3182 |
| TRP239\|236 | −0.1326 | 0.6571 |
| PRO241\|238 | −0.0703 | 0.4000 |
| ARG26\|23 | −0.9434 | 0.1429 |
| Putative Functional Cluster 8 | | |
| TYR113\|110 | 1.3591 | 0.7451 |
| GLU277\|601 | −0.2486 | 0.3000 |
| TRP278\|602 | 0.5985 | 1.0000 |
| ALA315\|312 | 0.4810 | 1.0000 |
| GLY316\|313 | 0.1753 | 0.5385 |
| VAL317\|314 | −0.7427 | 0.8276 |
| TRP318\|315 | −0.6090 | 1.0000 |
| ARG323\|320 | −0.7533 | 0.5135 |
| LEU329\|326 | −0.6071 | 0.5000 |
| GLN33\|357 | 0.0467 | 0.6304 |
| LEU330\|327 | −0.2544 | 0.6538 |
| PRO35\|359 | 0.5221 | 0.6444 |
| ILE36\|360 | 0.8911 | 1.0000 |
| LEU37\|361 | 0.1502 | 1.0000 |
| SER38\|362 | −0.3957 | 0.6571 |
| ARG39\|363 | −0.5489 | 0.6154 |
| ASP42\|366 | −0.4830 | 0.4706 |
| THR44\|368 | −0.6736 | 0.4211 |
| TYR5\|2 | −0.3166 | 0.2973 |
| VAL70\|394 | 0.0393 | 1.0000 |
| LEU73\|397 | 0.2238 | 1.0000 |
| TRP76\|400 | −0.2611 | 0.7619 |
| GLN79\|403 | −0.3008 | 0.5882 |
| THR80\|404 | −0.9104 | 0.7200 |
| GLN83\|407 | −0.9741 | 0.2727 |
| Putative Functional Cluster 9 | | |
| ARG100\|97 | 1.0756 | 0.7174 |
| VAL114\|111 | 1.5269 | 0.7297 |
| ASP115\|112 | 1.4782 | 0.6279 |
| GLN116\|113 | 2.1759 | 0.6061 |
| ASP118\|115 | 2.4211 | 1.0000 |
| GLU186\|183 | 1.0593 | 0.7931 |
| ILE201\|198 | 1.8787 | 1.0000 |
| ARG214\|211 | 1.4141 | 0.6364 |
| ALA215\|212 | 1.7829 | 1.0000 |
| TYR218\|215 | 1.8645 | 0.7660 |
| ASP219\|216 | 2.4473 | 1.0000 |
| ASP235\|232 | 1.9633 | 1.0000 |
| LEU237\|234 | 1.1109 | 1.0000 |
| GLU248\|245 | 1.6440 | 0.5957 |
| LEU249\|246 | 1.4163 | 1.0000 |
| SER250\|247 | 2.3789 | 1.0000 |
| SER251\|248 | 2.3222 | 0.6905 |
| ARG255\|252 | 2.4824 | 1.0000 |
| GLY293\|290 | 2.8630 | 0.6190 |
| GLY294\|291 | 2.8340 | 0.7692 |
| GLY296\|293 | 2.2937 | 0.7931 |
| ARG299\|296 | 1.6386 | 0.5250 |
| ILE310\|307 | 1.3041 | 1.0000 |
| ASP46\|43 | 2.0995 | 1.0000 |
| LYS77\|74 | 2.2322 | 1.0000 |

Table 5 lists the nine largest putative functional clusters among the 63 putative functional clusters identified on the surface of PDB:12asA.

Table 6 details the highest scoring of the nine putative functional clusters identified in Table 5, the residues that comprise this putative functional cluster, listed under the heading “Residue Id.”, their residue conservation z-scores, listed under the heading “Residue Cons. Z-score”, and their corresponding surface orientation scores, listed under the heading “Surface Orient. Scores”. Beneath the residue listing are the components of the four dimensional functional annotation score vector that represents this putative functional cluster. “Csv” Score refers to the z-score of the highest neighbor averaged residue conservation score. ‘Volume’ refers to the volume of the functional cluster. “Mouth area” refers to the area of the putative functional cluster's mouth. “Depth” refers to the depth of the putative functional cluster. “Cont SVM Score” refers to continuous SVM score characterizing this putative functional cluster.

| | Residue | Surface |
|---|---|---|
| Residue | Cons. | Orient. |
| Id. | Z-score | Score |
| Putative Functional Cluster 6 | | |
| ARG100\|424 | 1.2179 | 0.7143 |
| SER111\|435 | 1.0295 | 1.0000 |
| VAL114\|438 | 1.5666 | 0.7143 |
| ASP115\|439 | 1.1860 | 0.7660 |
| GLN116\|440 | 2.6940 | 0.5862 |
| ASP118\|442 | 2.8645 | 1.0000 |
| GLU186\|510 | 0.8919 | 0.7188 |
| LEU198\|522 | 1.5269 | 1.0000 |
| ILE201\|525 | 1.8921 | 1.0000 |
| HIS211\|535 | 0.8670 | 1.0000 |
| ARG214\|538 | 1.3341 | 0.5870 |
| ALA215\|539 | 1.6168 | 1.0000 |
| TYR218\|542 | 1.5988 | 0.6977 |
| ASP219\|543 | 2.3869 | 1.0000 |
| ASP235\|559 | 1.8816 | 0.9211 |
| LEU237\|561 | 1.2497 | 1.0000 |
| ALA246\|570 | 0.8437 | 1.0000 |
| GLU248\|572 | 1.7245 | 0.6429 |
| LEU249\|573 | 2.3089 | 1.0000 |
| SER250\|574 | 1.9222 | 1.0000 |
| SER251\|575 | 2.2703 | 0.6591 |
| ARG255\|579 | 2.5792 | 1.0000 |
| GLY293\|617 | 3.0107 | 0.7000 |
| GLY294\|618 | 2.9080 | 0.7200 |
| GLY296\|620 | 2.4335 | 0.7667 |
| ARG299\|623 | 1.7470 | 0.7027 |
| ILE310\|634 | 1.2665 | 1.0000 |
| GLN314\|638 | 1.2677 | 1.0000 |
| ASP46\|370 | 2.1090 | 1.0000 |
| LYS77\|401 | 2.8404 | 1.0000 |

Cont-SVM Score: 1.2009
Csv Score: 2.8630
Volume: 234.9285 A³
Mouth Area: 153.9238A²
Depth: 3.5997 A

Table 6 details the highest scoring functional cluster on PDB:12asA.

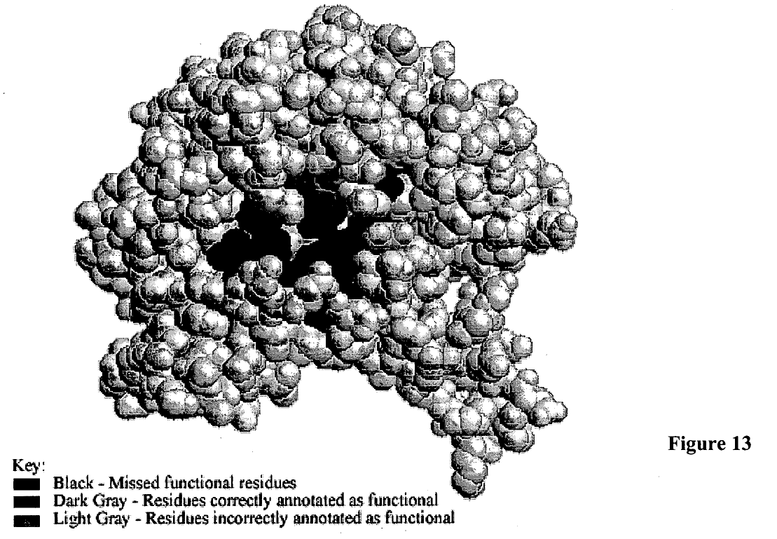

FIG. 13 illustrates this putative functional cluster. The black residues indicate those residues that are missed by the algorithm, the dark gray residues indicate those residues that are correctly predicted, and the light gray residues indicate incorrectly predicted residues (false positives).

Method for Determining the Probability that a Putative Functional Cluster is a Functional Cluster Using Continuous SVM Scores or Other Functional Annotation Scores.

Another aspect of the invention is a method for determining the probability, or confidence, that a putative functional cluster characterized by a continuous SVM score, is in fact functional. As one ordinarily skilled in the art would appreciate a continuous SVM score is one type of a functional annotation score. Accordingly, the following method may be generalized to any functional annotation scoring scheme. This aspect of the invention is based upon the recognition that the PDB co-crystallographic record may be used as an experimentally verified standard for the backtesting the accuracy of computational methods for identifying and representing putative functional clusters. Other suitable standards include any current or future, public or proprietary, databases of protein structures containing annotated functional sites.

One method for determining the probability that a putative functional cluster, characterized by a corresponding functional annotation score, is functional comprises the steps of: 1) selecting a plurality of reference proteins, each comprising a validated functional cluster; 2) for each reference protein, identifying one or more reference functional clusters using the same method that was used to identify said putative functional cluster; 3) for each reference functional cluster that was identified in step 2), determining a corresponding functional annotation score of the same type that was used to characterize said putative functional; 4) determining the fraction of reference functional clusters identified in step 2) that correctly correspond to validated functional clusters identified in step 1) at each functional annotation score, for a plurality of functional annotation scores; and 5) identifying the probability that said putative functional cluster is functional with the fraction of reference functional clusters, characterized by a functional annotation scores that are each equal to the functional annotation score of said putative functional cluster, correctly identified as corresponding to validated functional clusters in step 4).

The first step in this method selects a plurality of reference proteins; each protein comprising one or more validated functional clusters. One embodiment of the invention uses all of the PDB co-crystals for a “plurality of reference proteins”. The recitation to a “plurality of reference proteins” is intended to recognize that depending upon the accuracy required by a user, there is no general limitation on the number of reference proteins that must be utilized. It is also intended to represent that if a functional annotation method is applied to putative functional clusters from one particular protein family, a minimum probability may be calculated by using those reference structures from that particular protein family. For example, if putative functional clusters are drawn from only kinases, the determination of the probability that a putative functional cluster is functional may only consider use reference proteins that are kinases.

The second step in the method backtests the method used to identify the putative functional cluster of interest by using it to identify reference functional clusters on the reference proteins selected in step 1). As used herein a “reference functional cluster” refers to a validated functional cluster that has been “re-identified” using a functional annotation method for the purposes of backtesting the accuracy of the functional annotation method. Any of the method disclosed herein for identifying putative functional reference clusters and putative functional clusters may be used for identifying reference functional clusters. A reference functional cluster is correctly identified if it contains at least a lower threshold percentage and no more than an upper threshold percentage of the residues that comprise the validated functional cluster it corresponds to. Methods according to this aspect of the invention may use a lower threshold as low as 35% and a upper threshold as high as 65%—i.e. a reference functional cluster is identified as such if it comprises more than 0.35N and less than 1.65N, where N is the number of residues of its corresponding validated functional cluster. Alternatively, methods according to this aspect of the invention may use only a lower threshold—i.e. a reference functional cluster is considered correctly identified if it comprises more than 0.35N.

The third step in this method determines a functional annotation score for each reference functional cluster of the same type that characterizes the putative functional cluster of interest. Putative functional clusters and reference functional cluster may be characterized by any functional annotation score disclosed herein, known in the art, or later developed in the art. One embodiment according to the invention uses a continuous SVM score according to Equation 8 as a functional annotation score. As one ordinarily skilled in the art will appreciate, while it is preferable to determine a functional annotation score for each reference functional annotation, functional annotations scores may be determined for a subset of the reference functional clusters, if less accuracy is required.

The fourth step in this method determines the fraction of reference functional cluster identifications that correctly correspond to validated functional clusters at each functional annotation score for a plurality of functional annotation scores.

The last step in this method identifies the probability that putative functional cluster is in fact functional with the fraction of reference functional clusters, characterized by a functional annotation scores that are each equal to the functional annotation score of said putative functional cluster, correctly identified as corresponding to validated functional clusters in step four. This aspect of the invention will be illustrated in the following example.

### EXAMPLE

### Determining the Probability that the Highest Scoring Putative Functional Cluster Identified on PDB:12asA is Functional

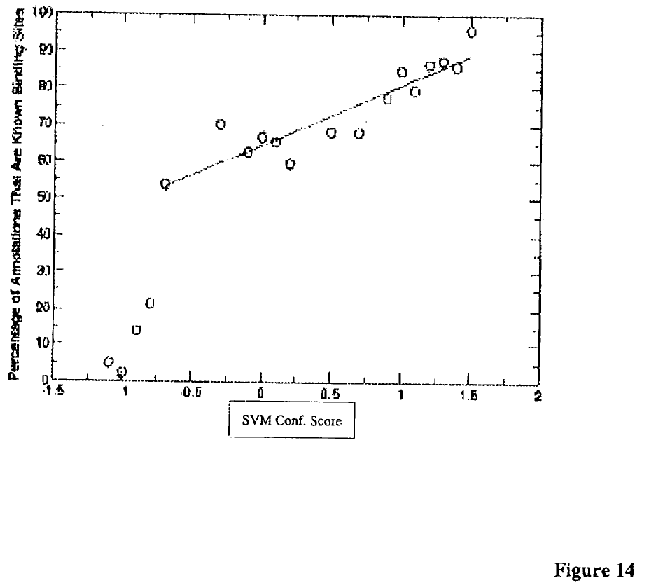

The methods illustrated in FIGS. 1b and 4 were implemented as described in the section entitled, Example: Identifying the Functional Site on PDB:12asA and Determining its Corresponding Continuous SVM Score, to identify putative functional clusters, corresponding to known small molecule binding sites in a set of 1188 PDB co-crystal structures, and determine continuous SVM scores for each putative functional cluster. Continuous SVM scores according to Equation 8 were determined for 8,768 sites distributed among the 1188 co-crystal structures. FIG. 14 shows the percentage of correct annotations as a function of confidence scores. The solid line represents the optimal linear fit to the data using linear regression analysis. The highest point on the plot at (1.5, 95) implies that when the claimed methods assigned a score between 1.45 and 1.55, 95% of the sites it annotated are sites that exist in the crystallographic record. The lower threshold scores was 50%, there was no upper threhold. Thus, a particular annotation is considered correct if it comprises more than half of the residues that comprise the corresponding co-crystal structure. Because the crystallographic record does not contain all of the small molecule binding sites for these proteins, it is probable that the other 5% of the sites that the claimed methods find at this score threshold are also small molecule binding sites. The highest scoring putative functional cluster identified on 12asA is characterized by a continuous SVM score of 1.20. Accordingly, based upon the linear regression fit shown in FIG. 14, it has a minimum 75% probability of being a true functional cluster. Sites with scores between 1.45 and 1.55 have a minimum 95% likelihood of being a small molecule binding site. The rate of false positives does not increase significantly until the functional annotation score drops below −0.7, at which point the number of annotated sites not in the crystallographic record increases dramatically. On average, the claimed methods when so implemented find 1.4 reliable small molecule sites per protein (1690 sites on 1188 proteins).

### EXAMPLE

### Comparisons of the Methods According to FIG. 1b with PASS

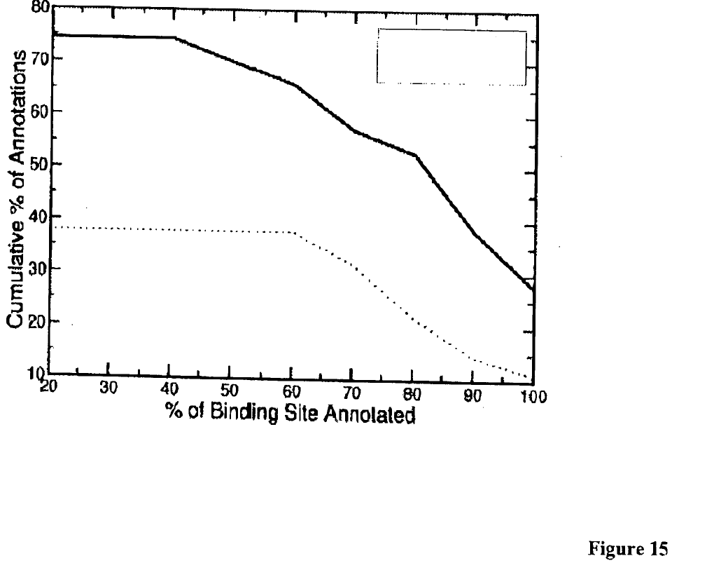

One of the most widely used algorithms for finding the small molecule binding sites in proteins is the PASS algorithm developed at DuPont Pharmaceuticals. FIG. 15 shows a comparison between the methods illustrated in FIG. 4 (solid line) and PASS (dashed line) on a set of 82 co-crystals. Reference functional clusters were identified using the methods illustrated in FIG. 4. A reference functional cluster is considered correctly identified if it comprises more than half of the residues that comprise the corresponding co-crystal structure. The claimed methods find more than 70% of the small molecule binding sites in the set, while PASS finds less than 40%. Further, the claimed methods annotate 80% of the residues involved in binding for more than 55% of the sites in the co-crystal set, while PASS annotates less than 25% to this degree of precision.

### EXAMPLE

### Further Exemplary Functional Site Identifications

The methods illustrated in FIGS. 1b and 4 were implemented as described in the section entitled, Example: Identifying the Functional Site on PDB:12asA and Determining its Corresponding Continuous SVM Score, to test their ability to identify known small molecule binding sites that have been the subject of recent drug discovery efforts and determine continuous SVM scores for each site identification. Continuous SVM scores were converted into probabilities that such site identifications are correct using the best fit line from FIG. 14 and the method used to generate it.

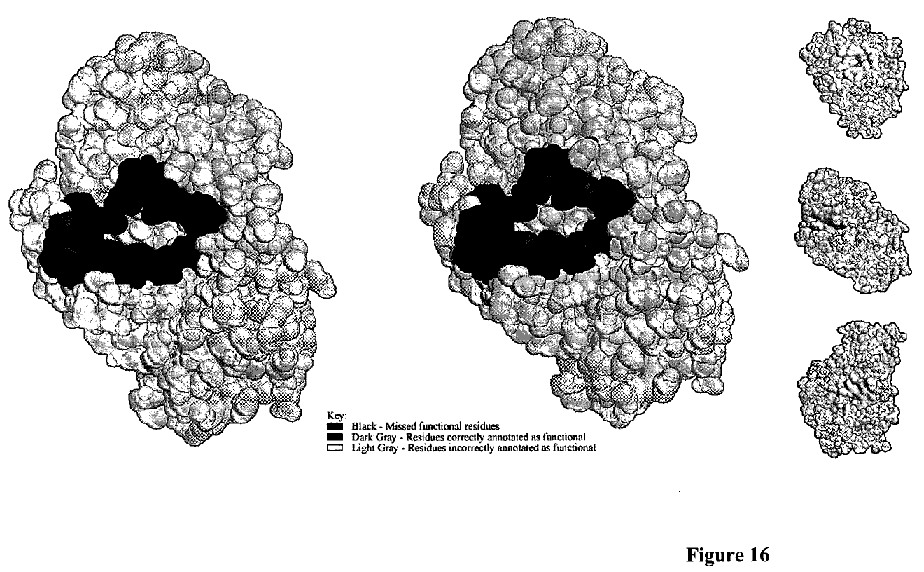

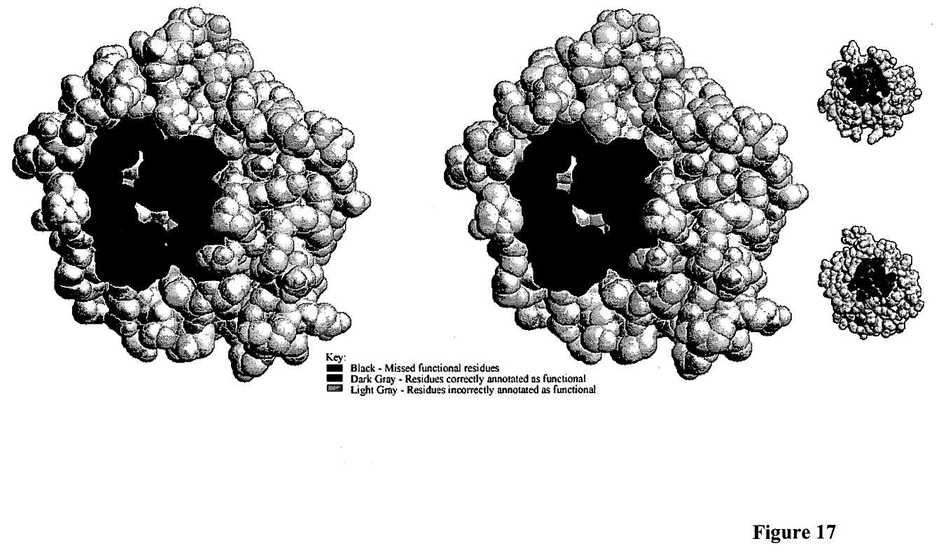

FIG. 16 compares the identification of the lead acetate binding site on Ferrochelatase (PDB:1HRK), with a greater than 80% probability that the annotation is correct, using the methods according to the invention, (shown on the left), and the top four identifications made by the state-of-the-art PASS algorithm (shown on the right). The true inhibitor site (PDB:1HRK) ranks 4^{th} among the PASS identifications. The dark residues indicate those residues that are missed by the algorithm, the dark gray residues indicate those residues that are correctly predicted, and the light gray residues indicate those residues that are incorrectly predicted (false positives). The same residue coloring scheme will be used for FIGS. 17-21. PASS correctly annotates very few of the lead acetate binding residues. Ferrochelatase is responsible for catalyzing the rate-limiting step of heme biosynthesis. When Ferrochelatase is inhibited, its substrate, the chemical photosensitizer PpIX, accumulates in the cell. Higher levels of PpIX are directly correlated to the improved efficacy of a new cancer treatment, photodynamic therapy (PDT). PDT in combination with the Ferrochelatase inhibitor, lead acetate, has been shown to cause almost complete regression of cutaneous tumors in mice. However, since lead acetate is a highly toxic compound, safer and non-toxic Ferrochelatase inhibitors are needed if this is to become an effective strategy for human cancer treatment. Structural information on the active site of Ferrochelatase will greatly aid the design of new, non-toxic inhibitors.

FIG. 17 compares the identification of the novel Lovastatin binding site on Lymphocyte Function Associated Antigen-1 (PDB:1CQP), with a greater than 95% probability that the annotation is correct, using the methods according to the invention (shown on the left) and the top three identifications made by the state-of-the-art PASS algorithm (shown on the right). The true inhibitor site as determined from the co-crystal (PDB:1CQP) ranks 3^{rd} among the PASS identifications. While the methods according to the invention annotate almost all lovastatin binding residues correctly, PASS annotates very few. The interaction between LFA-1 and ICAM-1 is a key signaling event in the inflammatory process, and thus inhibitors of this interaction are currently being pursued. Recently, in a small-molecule high-throughput screen at Novartis, Inc., Lovastatin was identified as an inhibitor of LFA-1 mediated adhesion of leukocytes to ICAM-1. Lovastatin (Mevacor) is a member of the statin class of HMG-CoA reductase inhibitors. Statins are the most commonly prescribed class of cholesterol-reducing drugs and collectively generate annual sales in excess of $15 billion. The crystal structure of LFA-1 in complex with Lovastatin shows that the statin-binding site on LFA-1 is distant from the ICAM-1 binding region, and represents a novel site for small-molecule inhibition. The discovery that LFA-1 contains a binding site for the statins not only identifies a novel mechanism for inhibition of the LFA-1-ICAM-1 interaction, but ialso opens up an entirely new therapeutic opportunity for the statins in connection to anti-inflammatory applications. It may also be expected that the biological activity of any other binding sites that are highly homologous to this site on LFA-1 may also be mediated by Lovastatin.

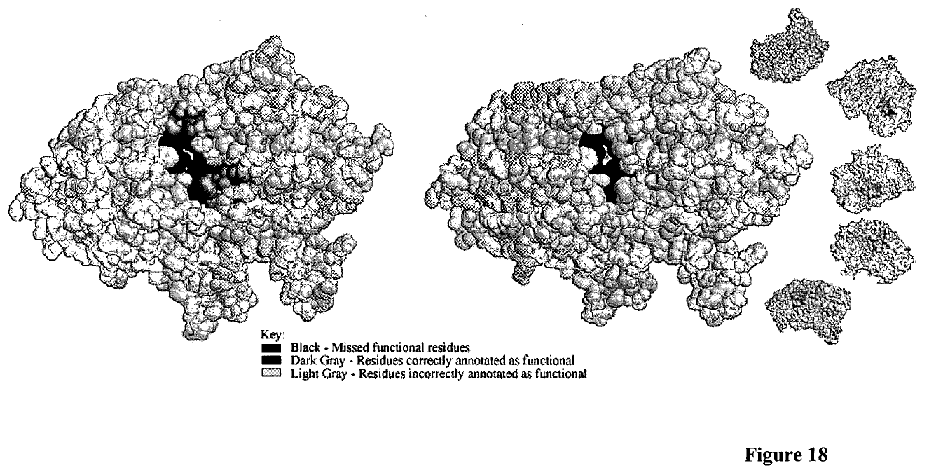

FIG. 18 compares the identification of the CP320626-binding site on Glycogen Phosphorylase B (“GBp”) (PDB:E1Y), with a greater than 85% probability that the annotation is correct, using the methods according to the invention (shown on the left), and the top six identifications made by the state-of-the-art PASS algorithm (shown on the right). The true inhibitor site as determined from the co-crystal (PDB:1H5U) ranks 6^{th} among the PASS identifications. Glycogen phosphorylase B (GPb) is a therapeutic target currently undergoing investigation for the treatment of diabetes. Recently, in a high-throughput screen for small-molecule inhibitors of GPb, researchers at Pfizer, Inc., identified a potent GPb inhibitor. Based on the structure of this hit, the lead compound CP320626 was synthesized, which potently inhibits GPb in a non-competitive manner and has no apparent structural relation to any of the physiological ligands of GPb. The structure of a GPb-CP320626 complex was subsequently determined in order to reveal the binding site of CP320626. A new allosteric binding site was identified that is spatially distinct from the catalytic and effector sites of GPb. Despite binding in a region that is far from the catalytic site, CP320626 is able to effectively reduce the enzymatic activity by promoting the less active T-state conformation of GPb over the active R-state conformation. This new binding site represents a new target for structure-based design of novel anti-diabetes compounds with improved pharmacological properties. It may also be expected that the biological activity any other binding sites that are highly homologous to this site on GPb may also be mediated by CP320626.

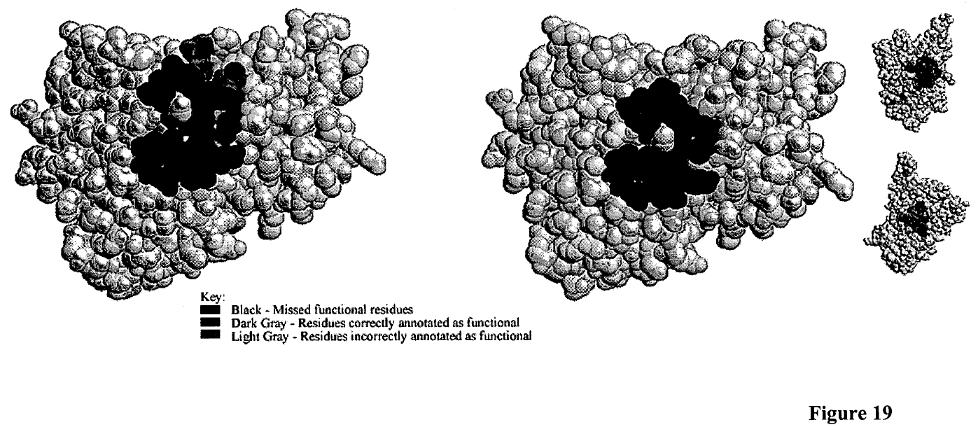

FIG. 19 compares the identification of the anilinoquinazoline binding site on Fructose 1-6-Biphosphatase (“FBPase”) (PDB:1KZ8), with a greater than 80% probability that the annotation is correct, using the methods according to the invention (shown on the left), and the top three identifications made by the state-of-the-art PASS algorithm (shown on the right). The true inhibitor site as determined from the co-crystal (PDB:1KZ8), ranks 3^{rd} among the PASS identifications. FBPase is one of the rate-limiting enzymes of hepatic gluconeogenesis, and its expression is significantly upregulated in Type 2 diabetes. FBPase inhibitors should lower blood glucose by inhibiting the elevated rate of gluconeogenesis, however no clinically useful FBPase inhibitors have yet been developed. Some inhibitors that have been investigated include substrate-competitive inhibitors that bind to the F6P binding site, and allosteric inhibitors that bind to the AMP binding site. In a recent screen for novel allosteric FBPase inhibitors, researchers at Pfizer, Inc., discovered an anilinoquinazoline inhibitor that did appear to bind to either of the known binding sites of FBPase. Co-crystallization of the inhibitor with FBPase led to the discovery of a novel allosteric binding site, distinct from both the AMP and F6P sites. The discovery of this allosteric site represents an entirely new approach to the inhibition of FBPase and allows a novel class of compounds to be pursued for further drug design. It may also be expected that the biological activity pf any other binding sites that are highly homologous to this site on FBPase, may also be mediated by anilinoquinazoline.

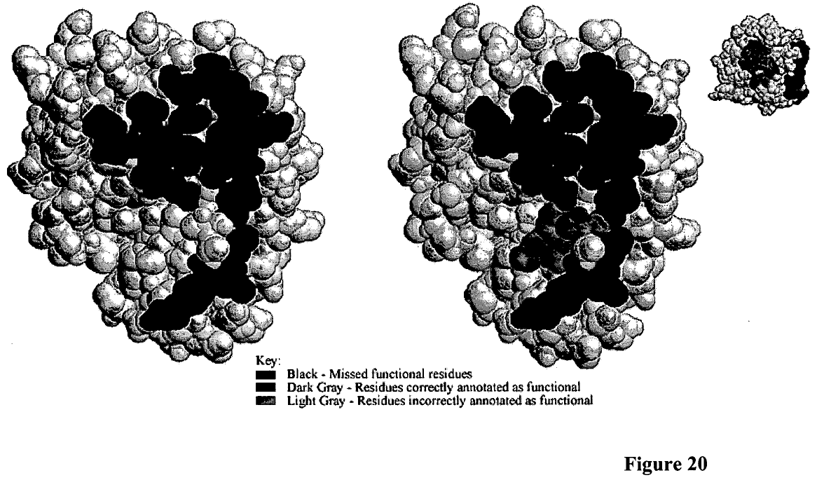

FIG. 20 compares the identification of the peptide exo-site on Factor VIIa (PDB:1KLJ), with a greater than 65% probability that the annotation is correct, using the methods according to the invention (shown on the left), and the top three identifications made by the state-of-the-art PASS algorithm (shown on the right). The true inhibitor site as determined from the co-crystal (PDB:1DVA) ranks 2^{nd} among the PASS identifications.

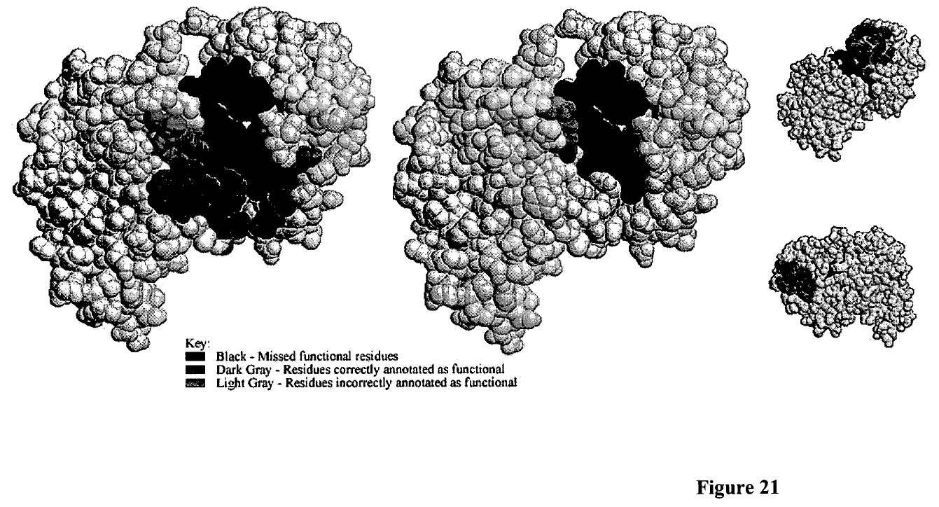

FIG. 21 compares the identification of the binding site on the P38 Kinase (PDB:1A9U), with a greater than 85% probability that the annotation is correct, using the methods according to the invention (shown on the left), and the top three identifications made by the state-of-the-art PASS algorithm (shown on the right). The true inhibitor site as determined from the co-crystal (PDB:1KV2) ranks 3^{rd} among the PASS identifications.

Systems According to the Invention.

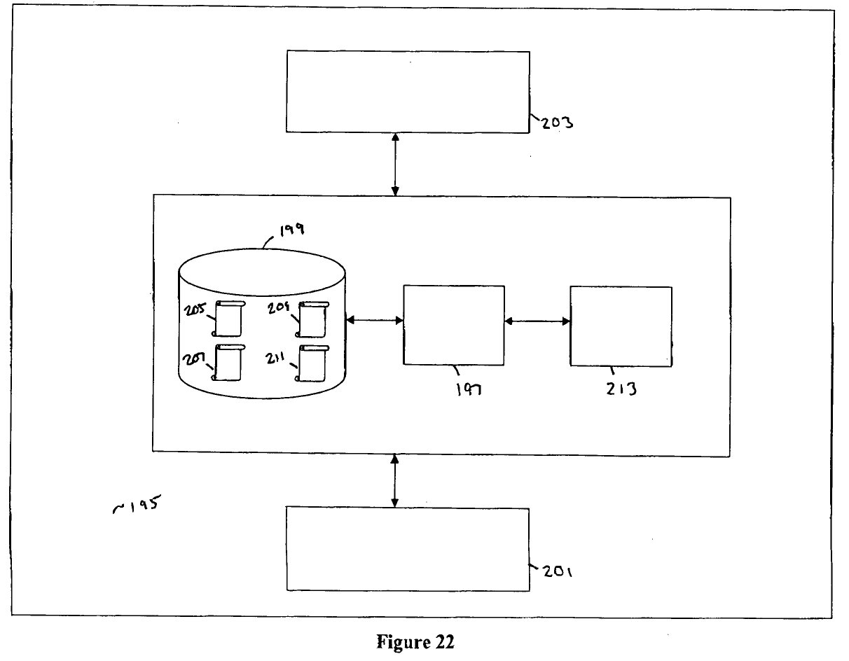

In general, as is shown in FIG. 22, a system according to the invention 195 comprises a processor 197, a memory 199, optionally, an input device 201, optionally, an output device 203, programming for an operating system 205, programming for the methods according to the invention 207, optionally, programming for displaying protein structures based upon their structural coordinates 209, and optionally, programming for storing and retrieving a plurality of sequences and structures 211. The systems according to the invention may optionally, also comprise a device for networking to another device 213.

A processor 197, as used herein, may include one or more microprocessor(s), field programmable logic array(s), or one or more applications specific integrated circuit(s). Exemplary processors include, but are not limited to, Intel Corp.'s Pentium series processor (Santa Clara, Calif.), Motorola Corp.'s PowerPC processors (Schaumberg, Ill.), MIPS Technologies Inc.'s MIPs processors (Mountain View, Calif.), or Xilinx Inc.'s Vertex series of field programmable logic arrays (San Jose, Calif.).

A memory 199, as used herein, is any electronic, magnetic or optical based media for storing, reading and writing digital information or any combination of such media. Exemplary types of memory include, but are not limited to, random access memory, electronically programmable read-only memory, flash memory, magnetic based disk and tape drives, and optical based disk drives. The memory stores: 1) programming for the methods according to the invention 207; 2) programming for displaying protein structures based upon their structural coordinates 209; 3) programming for an operating system 205; and 4) programming for storing and retrieving a plurality of sequences and structures 211.

An input device 201, as used herein, is any device that accepts and processes information from a user. Exemplary devices include, but are not limited to, a keyboard and mouse, a touch screen/tablet, a microphone, any removable, optical, magnetic or electronic media based drive, such as a floppy disk drive, a removable hard disk drive, a Compact Disk/Digital Video Disk drive, a flash memory reader, or any combination thereof.

An output device 203, as used herein, is any device that processes and outputs information to a user. Exemplary devices include, but are not limited to, visual displays, speakers and or printers. A visual display may be based upon any technology known in the art for processing and presenting a visual image to a user, including, cathode ray tube based monitors/projectors, plasma based monitors, liquid crystal display based monitors, digital micro-mirror device based projectors, or light-valve based projectors.

Programming for an operating system 205, as used herein, refers to any machine code, executed by the processor 197, for controlling and managing the data flow between the processor 197, the memory 199, the input device 201, the output device 203, and any networking devices 213. In addition to managing data flow among the hardware components that comprise a computer system, an operating system also provides, scheduling, input-output control, file and data management, memory management, and communication control and related services, all in accordance with known methodologies. Exemplary operating systems include, but are not limited to, Microsoft Corp.'s Windows and NT (Redmond, Wash.), Sun Microsystems, Inc.'s Solaris Operating System (Palo Alto, Calif.), Red Hat Corp.'s version of Linux (Durham, N.C.) and Palm Corp.'s PALM OS (Milpitas, Calif.).

Programming for displaying protein structures based upon their structural coordinates 209, as used herein, refers to machine code, that when executed by the processor, displays protein structures to the user via the output device, 203, based upon their structural coordinates. Exemplary software for displaying protein structures includes but is not limited to, Rasmol, available for download at http://www.rasmol.org/, Cn3D available for download at http://www.ncbi.nlm.nih.gov/Structure/CN3D/cn3d.shtml, Molscript, available for download at, http://www.avatar.se/molscript/, MolMol available for download at http://www.mol.biol.ethz.ch/wuthrich/software/molmol/, and the Insight II software suite available from Accelrys, Inc., (San Diego, Calif.). An input file comprising a query structure with an identification of its functional residues must be formatted based upon the particular protein viewer that is being employed. This is well within the capacity of one ordinarily skilled in the art. For those current or future viewers that recognize PDB site records, one method would input the query structure and functional residue identifications in PDB format with the functional residue identifications denoted as site records. See http://www.rcsb.org/pdb/docs/format/pdbguide2.2/guide2.2 frame.html. For those current or future viewers that do not recognize PDB site records, but recognize other PDB file formats, a script may be written, using either the native scripting features in a viewer, or in an external scripting language, to “select” the functional residues in the query structure file for highlighting in the display.

Programming for storing and retrieving a plurality sequences and structures 211, as used herein, refers to machine code, that when executed by the processor, allows for the storing, retrieving, and organizing of a plurality of sequences and structures. Exemplary software includes relational and object oriented databases such as Oracle Corp.'s 9i (Redwood City, Calif.), International Business Machine, Inc.'s, DB2 (Armonk, N.Y.), Microsoft Corp.'s Access (Redmond, Wash.) and Versant Corp.'s, Versant Developer Suite 6.0 (Freemont, Calif.). If structures and sequences are stored as flat files, programming for storing and retrieving a plurality of structures and sequences includes programming for operating systems.

Programming for the methods according to the invention 207, as used herein, refers to machine code, that when executed by the processor, performs the methods according to the invention. The source code/object code may be written in any current programming language, such as JAVA or C++, or any future programming language.

A networking device 213 as used herein refers to a device that comprises the hardware and software to allow a system according to the invention to electronically communicate either directly or indirectly to a network server, network switch/router, personal computer, terminal, or other communications device over a distributed communications network. Exemplary networking schemes may be based on packet over any media and include but are not limited to, Ethernet 10/100/1000, IEEE 802.11x, SONET, ATM, IP, MPLS, IEEE 1394, xDSL, Bluetooth, or any other ANSI approved standard.

It will be appreciated by one skilled in the art that the programming for an operating system 205, programming for displaying protein structures based upon their structural coordinates 209, programming for storing and retrieving a plurality of sequences and structures 211, and the programming for the methods according to the invention 207 may be loaded on to a system according to the invention through either the input device 201, a networking device 213, or a combination of both.

Systems according to the invention may be based upon personal computers (“PCs”) or network servers programmed to perform the methods according to the invention. A suitable server and hardware configuration is an enterprise class Pentium based server, comprising an operating system such as Microsoft's NT, Sun Microsystems' Solaris or Red Hat's version of Linux with IGB random access memory, 100 GB storage, either a line area network communications card, such as a 10/100 Ethernet card or a high speed Internet connection, such as a T1/E1 line, optionally, an enterprise database, programming for the methods according to the invention and optionally, programming for displaying protein structures. The storage and memory requirements listed above are not intended to represent minimum hardware configurations, rather they represent a typical server system which may readily purchased from vendors at the time of filing. Such servers may be readily purchased from Dell, Inc. (Austin, Tex.), or Hewlett-Packard, Inc., (Palo Alto, Calif.) with all the features except for the enterprise database, programming for displaying protein structures based upon their structural coordinates, and the programming for the methods according to the invention. Enterprise class databases may be purchased from Oracle Corp. or International Business Machines, Inc. It will be appreciated by one skilled in the art that one or more servers may be networked together. Accordingly, the programming for the methods according the invention and the enterprise database may be stored on physically separate servers in communication with each other. Programming for displaying protein structures based upon their structural coordinates may be purchased from Accelrys, Inc. (San Diego, Calif.) or downloaded from the links provided above and installed on an enterprise server. It will further be appreciated by one skilled in the art that a network server need not include programming for displaying protein structures based upon their structural coordinates, if the client comprises such programming.

A suitable desktop PC and hardware configuration is a Pentium based desktop computer comprising at least 128 MB of random access memory, 10 GB of storage, a Windows or Linux based operating system, optionally, either a line area network communications card, such as a 10/100 Ethernet card or a high speed Internet connection, such as a T1/E1 line, optionally, a TCP/IP web browser, such as Microsoft's Internet Explorer or the Mozilla Web Browser, optionally, a database such as Microsoft's Access, programming for displaying protein structures, and programming for the methods according to the invention. Once again, the exemplary storage and memory requirement are only intended to represent PC configurations which are readily available from vendors at the time of filing. They are not intended to represent minimum configurations. Such PCs may be readily purchased from Dell, Inc. or Hewlett-Packard, Inc., (Palo Alto, Calif.) with all the features except for the programming for displaying protein structures and the programming for the methods according to the invention.

Programming for displaying protein structures based upon their structural coordinates may be purchased from Accelrys, Inc. (San Diego, Calif.) or downloaded from the links provided above and installed.

Although the invention has been described with reference to preferred embodiments and specific examples, it will be readily appreciated by those skilled in the art that many modifications and adaptations of the invention are possible without deviating from the spirit and scope of the invention. Thus, it is to be clearly understood that this description is made only by way of example and not as a limitation on the scope of the invention as claimed below. All references herein are hereby incorporated by reference.

1. A method comprising the steps of:
a. determining residue conservation scores for a plurality of reference residues;
b. identifying a cluster of connected reference residues;
c. determining the average residue conservation score of the residues that comprise said cluster;
d. determining the average residue conservation score of those residue that do not comprise said cluster; and
e. if the average determined in step c) is greater than the average determined in step d), selecting said cluster as a datum for one class of training data for use in a binary classification model adapted for identifying a cluster of functional residues on the surface of a query protein or adapted for determining a continuous SVM score of a cluster of residues on the surface of query protein.

2. The method of claim 1 wherein said residue conservation score is selected from the group consisting of the: residue conservation z-score; neighbor averaged residue conservation z-score; residue conservation p-score; or neighbor averaged residue conservation p-score.

3. A method comprising the steps of:
a. determining residue conservation scores for a plurality of query residues;
b. identifying a cluster of connected query residues;
c. determining the average residue conservation score of the residues that comprise said cluster;
d. determining the average residue conservation score of those residue that do not comprise said cluster; and
e. if the average determined in step c) is greater than the average determined in step d), selecting said cluster as a testing datum for use in a binary classification model adapted for identifying a cluster of functional residues on the surface of a query protein or adapted for determining a continuous SVM score for a cluster residues on the surface of a protein.

4. The method of claim 3 wherein said residue conservation score is selected from the group consisting of the: residue conservation z-score; neighbor averaged residue conservation z-score; residue conservation p-score; or neighbor averaged residue conservation p-score.

5. A method comprising the steps of:
a. identifying a void on the surface of a reference protein;
b. determining the volume of said void;
c. comparing the volume of said void to the volume of a water molecule; and
d. if the volume of said void is greater than the volume of a water molecule, selecting said cluster as a datum for one class of training data for use in a binary classification model adapted for identifying a cluster of functional residues on the surface of a query protein or adapted for determining a continuous SVM score of a cluster of residues on the surface of a protein.

6. A method comprising the steps of:
a. identifying a void on the surface of a query protein;
b. determining the volume of said void;
c. comparing the volume of said void to the volume of a water molecule; and
d. if the volume of said void is greater than the volume of a water molecule, selecting said cluster as a testing datum for use in a binary classification model adapted for identifying a cluster of functional residues on the surface of a query protein or adapated for determining an a continuous SVM score of a cluster of residues on the surface of a protein.

7. A method for comprising the steps of:
a. determining a three dimensional Delaunay tessilation of all or substantially of the reference residues of a reference structure based upon their three-dimensional coordinates;
b. determining the Alpha Shape of the reference residues from the Delaunay tessellation; and
c. identifying empty, connected Delaunay tetrahedrons, thereby identifying a void;
d. determining the volume of said void by summing the volume of the empty, connected Delaunay tetrahedrons determined in step c); and
e. if the volume of said void is greater than the volume of a water molecule, selecting said cluster as a datum for one class of training data for use in a binary classification model adapted for identifying a cluster of functional residues on the surface of a query protein or adapted for determining a continuous SVM score of a cluster of residues on the surface of a query protein.

8. A method comprising the steps of:
a. determining a three dimensional Delaunay tessilation of all or substantially of the query residues of a query structure based upon their three-dimensional coordinates;
b. determining the Alpha Shape of the query residues from the Delaunay tessellation; and
c. identifying empty, connected Delaunay tetrahedrons, thereby identifying a void;
d. determining the volume of said void by summing the volume of the empty, connected Delaunay tetrahedrons determined in step c); and
e. if the volume of said void is greater than the volume of a water molecule, selecting said cluster as a testing datum for use in a binary classification model adapted for identifying a cluster of functional residues on the surface of a query protein or adapted for determining a continuous SVM score of a cluster of residues on the surface of a query protein.

9. A method comprising the steps of:
a. determining residue conservation scores and surface orientation scores for a plurality of the residues on the surface of a reference protein;
b. identifying a cluster of connected reference residues;
c. determining the average residue conservation score of the residues that comprise said cluster;
d. determining the average residue conservation score of those residue that do not comprise said cluster; and
e. if the average determined in step c) is greater than the average determined in step d) and if the distribution of the surface orientation scores that characterize the residues that comprise the cluster indicates that the cluster is concave, selecting said cluster as a datum for one class of training data for use in a binary classification model adapted for identifying a cluster of functional residues on the surface of a query protein or adapted for determining a continuous SVM score of a cluster of residues on the surface of a query protein.

10. The method of claim 9 wherein said residue conservation score is selected from the group consisting of the: residue conservation z-score; neighbor averaged residue conservation z-score; residue conservation p-score; or neighbor averaged residue conservation p-score.

11. A method comprising the steps of:
a. determining residue conservation scores and surface orientation scores for a plurality of the residues on the surface of a query protein;
b. identifying a cluster of connected query residues;
c. determining the average residue conservation score of the residues that comprise said cluster;
d. determining the average residue conservation score of those residue that do not comprise said cluster; and
e. if the average determined in step c) is greater than the average determined in step d) and if the distribution of the surface orientation scores that characterize the residues that comprise the cluster indicates that the cluster is concave, selecting said cluster as a testing datum for use in a binary classification model adapted for identifying a cluster of functional residues on the surface of a query protein or adapted for determining a continuous SVM score of a cluster of residues on the surface of a query protein.

12. The method of claim 11 wherein said residue conservation score is selected from the group consisting of the: residue conservation z-score; neighbor averaged residue conservation z-score; residue conservation p-score; or neighbor averaged residue conservation p-score.

13. A method comprising the steps of:
a. determining residue conservation scores and surface orientation scores for a plurality of the solvent accessible residues on the surface of a reference protein;
b. determining the statistical distribution of the surface orientation scores;
c. determining the putative functional residue limit based upon the statistical distribution of surface orientation scores;
d. determining a first surface orientation score threshold and a first residue conservation score threshold;
e. identifying those residues that are characterized by residue conservation scores that are greater than the first residue conservation score threshold and surface orientation scores that are greater than the first surface orientation score threshold as putative functional residues, thereby determining the first pass putative functional residues;
f. identifying at least one cluster comprising connected first pass putative functional residues;
g. for each cluster which was identified, determining whether the number of first pass putative functional residues in the cluster exceed the putative functional residue limit; if it does not, selecting said cluster as a datum for one class of training data for use in a binary classification model adapted for identifying a cluster of functional residues on the surface of a query protein or adapted for determining a continuous SVM score of a cluster of residues on the surface of a query protein; otherwise determining a second surface orientation score threshold and a second residue conservation threshold score threshold;
h. identifying those residues that are characterized by residue conservation scores that are greater than the second residue conservation score threshold and surface orientation scores that are greater than the second surface orientation score threshold as putative functional residues, thereby determining the second pass putative functional residues;
i. identifying at least one cluster comprising connected second pass putative functional residues;
j. for each cluster which was identified, determining whether the number of second pass putative functional residues in the cluster exceed the putative functional residue limit; if it does not, selecting said cluster as a datum for one class of training data for use in a binary classification model adapted for identifying a cluster of functional residues on the surface of a query protein or adapted for determining a continuous SVM score of a cluster of residues on the surface of a query protein; otherwise determining a third surface orientation score threshold and a second residue conservation threshold score threshold; and
k. repeating steps h-j until no cluster may be identified that comprises more putative functional residues than the putative functional residue limit.

14. The method of claim 13 wherein said residue conservation score is selected from the group consisting of the: residue conservation z-score; neighbor averaged residue conservation z-score; residue conservation p-score; or neighbor averaged residue conservation p-score.

15. The method of claim 14 wherein said statistical distribution of surface orientation score is determined by a method comprising the steps of:
a. determining the range of statistical orientation scores determined in step b) of claim 13; and
b. partitioning the surface orientation scores among a plurality of statistical bins wherein the width of each statistic bin is a fraction of the range of the surface orientation scores determined in step a).

16. The method of claim 15 wherein said putative functional residue limit is determined from a method comprising the steps of:
a. selecting the statistical bin containing the greatest number of surface orientation scores among the statistical bins determined in step b) of claim 15 that are each centered about a concave surface orientation score; and
b. identifying the putative functional residue limit with the number of surface orientation scores contained within the statistical bin selected in step a).

17. A method comprising the steps of:
a. determining residue conservation scores and surface orientation scores for a plurality of the residues on the surface of a query protein;
b. determining the statistical distribution of the surface orientation scores;
c. determining the putative functional residue limit based upon the statistical distribution of surface orientation scores;
d. determining a first surface orientation score threshold and a first residue conservation score threshold;
e. identifying those residues that are characterized by residue conservation scores that are greater than the first residue conservation score threshold and surface orientation scores that are greater than the first surface orientation score threshold as putative functional residues, thereby determining the first pass putative functional residues;
f. identifying at least one cluster comprising connected first pass putative functional residues;
g. for each cluster which was identified, determining whether the number of first pass putative functional residues in the cluster exceeds the putative functional residue limit; if it does not, selecting said cluster as a testing datum for use in a binary classification model adapted for identifying a cluster of functional residues on the surface of a query protein or adapted for determining a continuous SVM score of a cluster of residues on the surface of a query protein; otherwise determining a second surface orientation score threshold and a second residue conservation threshold score threshold;
h. identifying those residues that are characterized by residue conservation scores that are greater than the second residue conservation score threshold and surface orientation scores that are greater than the second surface orientation score threshold as putative functional residues, thereby determining the second pass putative functional residues;
i. identifying at least one cluster comprising connected second pass putative functional residues;
j. for each cluster which was identified, determining whether the number of second pass putative functional residues in the cluster exceeds the putative functional residue limit; if it does not, selecting said cluster as a testing datum for use in a binary classification model adapted for identifying a cluster of functional residues on the surface of a query protein or adapted for determining a continuous SVM score of a cluster of residues on the surface of a query protein; and
k. repeating steps h-j until no cluster may be identified that comprises more putative functional residues than the putative functional residue limit.

18. The method of claim 17 wherein said residue conservation score is selected from the group consisting of the: residue conservation z-score; neighbor averaged residue conservation z-score; residue conservation p-score; or neighbor averaged residue conservation p-score.

19. The method of claim 18 wherein said statistical distribution of surface orientation score is determined by a method comprising the steps of:
a. determining the range of statistical orientation scores determined in step b) of claim 16; and
b. partitioning the surface orientation scores among a plurality of statistical bins wherein the width of each statistic bin is a fraction of the range of the surface orientation scores determined in step a).

20. The method of claim 19 wherein said putative functional residue limit is determined from a method comprising the steps of:
a. selecting the statistical bin containing the greatest number of surface orientation scores among the statistical bins determined in step b) of claim 19 that are each centered about a concave surface orientation score; and
b. identifying the putative functional residue limit with the number of surface orientation scores contained within the statistical bin selected in step a).

21. A method comprising the steps of:
a. determining residue conservation scores for a plurality of residues on the surface of a reference protein;
b. identifying a void on the surface of a reference protein;
c. determining the average of the residue conservation scores for the residues that comprise the void identified in step b);
d. determining the average residue conservation scores for the remaining residues that do not comprise the void identified in step b);
e. determining the volume of said void; and
f. if the volume of said void is greater than the volume of a water molecule and the average residue conservation score determined in step c) is greater than the average residue conservation score determined in step d), selecting said cluster as a datum for one class of training data for use in a binary classification model adapted for identifying a cluster of functional residues on the surface of a query protein or adapted for determining a continuous SVM score of a cluster of residues on the surface of a query protein.

22. The method of claim 21 wherein said residue conservation score is selected from the group consisting of the: residue conservation z-score; neighbor averaged residue conservation z-score; residue conservation p-score; or neighbor averaged residue conservation p-score.

23. A method comprising the steps of:
a. determining residue conservation scores for a plurality of residues on the surface of a query protein;
b. identifying a void on the surface of a query protein;
c. determining the average of the residue conservation scores for the residues that comprise the void identified in step b);
d. determining the average residue conservation scores for the remaining residues that do not comprise the void identified in step b);
e. determining the volume of said void; and
f. if the volume of said void is greater than the volume of a water molecule and the average residue conservation score determined in step c) is greater than the average residue conservation score determined in step d), selecting said cluster as a testing datum for use in a binary classification model adapted for identifying a cluster of functional residues on the surface of a query protein or adapted for determining a continuous SVM score of a cluster of residues on the surface of a query protein.

24. The method of claim 23 wherein said residue conservation score is selected from the group consisting of the: residue conservation z-score; neighbor averaged residue conservation z-score; residue conservation p-score; or neighbor averaged residue conservation p-score.

25. A method comprising the steps of:
a. determining residue conservation scores for a plurality of residues on the surface of a reference protein;
b. determining a three dimensional Delaunay tessilation of all or substantially of the reference residues of said reference structure based upon their three-dimensional coordinates;
c. determining the Alpha Shape of the reference residues from the Delaunay tessellation; and
d. identifying empty, connected Delaunay tetrahedrons, thereby identifying a void;
e. determining the average of the residue conservation scores for the residues that comprise the void identified in step d);
f. determining the average residue conservation scores for the remaining residues that do not comprise the void identified in step d);
g. determining the volume of said void by summing the volume of the empty, connected Delaunay tetrahedrons determined in step d); and
h. if the volume of said void is greater than the volume of a water molecule and the average residue conservation score determined in step e) is greater than the average residue conservation score determined in step f), selecting said cluster as a datum for one class of training data for use in a binary classification model adapted for identifying a cluster of functional residues on the surface of a query protein or adapted for determining a continuous SVM score of a cluster of residues on the surface of a query protein.

26. The method of claim 25 wherein said residue conservation score is selected from the group consisting of the: residue conservation z-score; neighbor averaged residue conservation z-score; residue conservation p-score; or neighbor averaged residue conservation p-score.

27. A method comprising the steps of:
a. determining residue conservation scores for a plurality of residues on the surface of a query protein;
b. determining a three dimensional Delaunay tessilation of all or substantially of the query residues of said query structure based upon their three-dimensional coordinates;
c. determining the Alpha Shape of the query residues from the Delaunay tessellation; and
d. identifying empty, connected Delaunay tetrahedrons, thereby identifying a void;
e. determining the average of the residue conservation scores for the residues that comprise the void identified in step d);
f. determining the average residue conservation scores for the remaining residues that do not comprise the void identified in step d);
g. determining the volume of said void by summing the volume of the empty, connected Delaunay tetrahedrons determined in step d); and
h. if the volume of said void is greater than the volume of a water molecule and the average residue conservation score determined in step e) is greater than the average residue conservation score determined in step f), selecting said cluster as a testing datum for use in a binary classification model adapted for identifying a cluster of functional residues on the surface of a query protein or adapted for determining a continuous SVM score of a cluster of residues on the surface of a query protein.

28. The method of claim 27 wherein said residue conservation score is selected from the group consisting of the: residue conservation z-score; neighbor averaged residue conservation z-score; residue conservation p-score; or neighbor averaged residue conservation p-score.

29. A method comprising the step of selecting a validated functional cluster as a testing datum for use in a binary classification model adapted for identifying a cluster of functional residues on the surface of a query protein or adapted for determining a continuous SVM score of a cluster of residues on the surface of a query protein.

30. The method of claim 29 wherein said validated functional cluster is determined by identifying those residues in a protein-ligand structure whose solvent accessible surface area increases upon removal of the ligand.

31. A method for identifying a putative functional cluster comprising the steps of:
a. determining residue conservation scores and surface orientation scores for a plurality of the residues on the surface of a query protein;
b. identifying a cluster of connected query residues;
c. determining the average residue conservation score of the residues that comprise said cluster;
d. determining the average residue conservation score of those residue that do not comprise said cluster; and
e. if the average determined in step c) is greater than the average determined in step d) and if the distribution of the surface orientation scores that characterize the residues that comprise the cluster indicates that the cluster is concave, identifying said cluster as a putative functional cluster.

32. The method of claim 31 wherein said residue conservation score is selected from the group consisting of the: residue conservation z-score; neighbor averaged residue conservation z-score; residue conservation p-score; or neighbor averaged residue conservation p-score.

33. A method for identifying a putative functional cluster comprising the steps of:
a. determining residue conservation scores and surface orientation scores for a plurality of the residues on the surface of a query protein;
b. determining the statistical distribution of the surface orientation scores;
c. determining the putative functional residue limit based upon the statistical distribution of surface orientation scores;
d. determining a first surface orientation score threshold and a first residue conservation score threshold;
e. identifying those residues that are characterized by residue conservation scores that are greater than the first residue conservation score threshold and surface orientation scores that are greater than the first surface orientation score threshold as putative functional residues, thereby determining the first pass putative functional residues;
f. identifying at least one cluster comprising connected first pass putative functional residues;
g. for each cluster which was identified, determining whether the number of first pass putative functional residues in the cluster exceeds the putative functional residue limit; if it does not, identifying such a cluster as a putative functional cluster; otherwise determining a second surface orientation score threshold and a second residue conservation threshold score threshold;
h. identifying those residues that are characterized by residue conservation scores that are greater than the second residue conservation score threshold and surface orientation scores that are greater than the second surface orientation score threshold as putative functional residues, thereby determining the second pass putative functional residues;
i. identifying at least one cluster comprising connected second pass putative functional residues;
j. for each cluster which was identified, determining whether the number of second pass putative functional residues in the cluster exceeds the putative functional residue limit; if it does not, identifying such a cluster as a putative functional cluster; otherwise determining a third surface orientation score threshold and a second residue conservation threshold score threshold; and
k. repeating steps h-j until no cluster may be identified that comprises more putative functional residues than the putative functional residue limit.

34. The method of claim 33 wherein said residue conservation score is selected from the group consisting of the: residue conservation z-score; neighbor averaged residue conservation z-score; residue conservation p-score; or neighbor averaged residue conservation p-score.

35. The method of claim 34 wherein said statistical distribution of surface orientation score is determined by a method comprising the steps of:
a. determining the range of statistical orientation scores determined in step b) of claim 33; and
b. partitioning the surface orientation scores among a plurality of statistical bins wherein the width of each statistic bin is a fraction of the range of the surface orientation scores determined in step a).

36. The method of claim 35 wherein said putative functional residue limit is determined from a method comprising the steps of:
a. selecting the statistical bin containing the greatest number of surface orientation scores among the statistical bins determined in step b) of claim 33 that are each centered about a concave surface orientation score; and
b. identifying the putative functional residue limit with the number of surface orientation scores contained within the statistical bin selected in step a).

37. A method for determining a putative functional cluster comprising the steps of:
a. determining residue conservation scores for a plurality of residues on the surface of a query protein;
b. identifying a void on the surface of a query protein;
c. determining the average of the residue conservation scores for the residues that comprise the void identified in step b);
d. determining the average residue conservation scores for the remaining residues that do not comprise the void identified in step b);
e. determining the volume of said void; and
f. if the volume of said void is greater than the volume of a water molecule and the average residue conservation score determined in step c) is greater than the average residue conservation score determined in step d), identifying said void as a putative functional cluster.

38. The method of claim 37 wherein said residue conservation score is selected from the group consisting of the: residue conservation z-score; neighbor averaged residue conservation z-score; residue conservation p-score; or neighbor averaged residue conservation p-score.

39. A method for determining a putative functional cluster comprising the steps of:
a. determining residue conservation scores for a plurality of residues on the surface of a query protein;
b. determining a three dimensional Delaunay tessilation of all or substantially of the query residues of said reference structure based upon their three-dimensional coordinates;
c. determining the Alpha Shape of the query residues from the Delaunay tessellation; and
d. identifying empty, connected Delaunay tetrahedrons, thereby identifying a void;
e. determining the average of the residue conservation scores for the residues that comprise the void identified in step d);
f. determining the average residue conservation scores for the remaining residues that do not comprise the void identified in step d);
g. determining the volume of said void by summing the volume of the empty, connected Delaunay tetrahedrons determined in step d); and
h. if the volume of said void is greater than the volume of a water molecule and the average residue conservation score determined in step e) is greater than the average residue conservation score determined in step f), identifying said void as a putative functional cluster.

40. The method of claim 39 wherein said residue conservation score is selected from the group consisting of the: residue conservation z-score; neighbor averaged residue conservation z-score; residue conservation p-score; or neighbor averaged residue conservation p-score.

41. A method for identifying at least one cluster of functional residues on the surface of a query protein comprising the steps of:
a. identifying at least one validated functional cluster from at least one reference protein;
b. determining at least one putative functional reference cluster from at least one reference protein;
c. representing each validated functional cluster determined in step a) and each putative functional reference cluster determined in step b) with a functional annotation score of the same form;
d. identifying at least one putative functional cluster on the surface of a query protein;
e. representing each putative functional cluster determined in step d) with a functional annotation score of the same form as the functional annotation scores used to represent the putative functional reference clusters and the validated functional clusters in step c);
f. for each putative functional cluster identified in step d) comparing its functional annotation score determined in step e) to the functional annotation scores determined in step c); and
g. for each putative functional cluster identified in step d) determining whether it may be classified as a validated functional cluster, thereby identifying said putative functional cluster as a true functional cluster or whether it may be identified as putative functional reference cluster, thereby identifying said putative functional cluster as non-functional cluster, based upon the comparison made in step f).

42. The method of claim 41 wherein said functional annotation score is a one dimensional functional annotation score selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

43. The method of claim 42 wherein said functional annotation score is a multi-dimensional functional annotation score comprising one subscore selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

44. The method of claim 42 wherein in said functional annotation score is a multi-dimensional functional annotation score comprising:
a. a first subscore that reflects the residue conservation of a putative functional reference cluster, putative functional cluster or a validated functional cluster; and
b. a second subscore that reflects a topographic aspect that may be associated with a concave putative functional reference cluster, putative functional cluster or a validated functional cluster.

45. The methods of claim 44 wherein said first subscore is selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score;
and said second subscore is selected from the group consisting of the: cluster volume, cluster surface area, cluster “mouth” area, cluster “mouth” circumference, and cluster depth.

46. The method of claim 43 wherein said functional annotation score is a four dimensional functional annotation score consisting of the: cluster maximum residue conservation z-score, cluster surface area, cluster “mouth” area, and cluster depth.

47. The method of claim 45 wherein said comparison performed in step f) is made using a method selected from the group consisting of: support vector machines, Bayesian methods, neural network methods, and decision tree methods.

48. The method of claim 43 wherein said comparison performed in step f) is made using a method selected from the group consisting of: support vector machines, Bayesian methods, neural network methods, and decision tree methods.

49. The method of claim 45 wherein said comparison performed in step f) is made using a method selected from the group consisting of: support vector machines, Bayesian methods, neural network methods, and decision tree methods.

50. A method for identifying at least one cluster of functional residues on the surface of a query protein comprising the steps of:
a. identifying at least one validated functional cluster from at least one reference protein;
b. identifying at least one putative functional reference cluster on the surface of at least one reference protein;
c. representing each validated functional cluster identified in step a) and each putative functional reference cluster identified in step b) with a functional annotation score of the same form;
d. identifying at least one putative functional cluster on the surface of a query protein using;
e. representing each putative functional cluster determined in step d) with a functional annotation score of the same form as the functional annotation scores used to represent putative functional reference clusters and validated functional clusters in step b);
f. using a support vector machine to determine a hyperplane that defines a first set of functional annotation scores that characterize the validated functional clusters determined in step a) and that defines a second set of functional annotation scores that characterize the putative functional reference clusters determined in step b) based upon the functional annotation scores determined in step c);
g. determining for each functional annotation score determined in step e) whether it falls into the first set of functional annotation scores determined in step f) or falls into the second set of functional annotation scores determined in step f); and
h. for each functional annotation score identified in step g) as falling into the into the first set of functional annotation scores corresponding to the validated functional clusters, identifying the corresponding putative functional cluster as a functional cluster; for each functional annotation score identified in step g) as falling into the second set of functional annotation scores corresponding to the putative functional reference clusters, identifying the corresponding putative functional cluster as a non-functional cluster.

51. The method of claim 50 wherein said functional annotation score is a one dimensional functional annotation score selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

52. The method of claim 50 wherein said functional annotation score is a multi-dimensional functional annotation score comprising one subscore selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

53. The method of claim 50 wherein in said functional annotation score is a multi-dimensional functional annotation score comprising:
a. a first subscore that reflects the residue conservation of a putative functional reference cluster, putative functional cluster or a validated functional cluster; and
b. a second subscore that reflects a topographic aspect that may be associated with a concave putative functional reference cluster, putative functional cluster or a validated functional cluster.

54. The methods of claim 53 wherein said first subscore is selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score; and said second subscore is selected from the group consisting of: the: cluster volume, cluster surface area, cluster “mouth” area, cluster “mouth” circumference, and cluster depth.

55. The method of claim 50 wherein said functional annotation score is a four dimensional functional annotation score consisting of the: cluster maximum residue conservation z-score, cluster surface area, cluster “mouth” area, and cluster depth.

56. The method of claim 52 wherein said putative functional reference clusters are determined using the method of claim 16 and said putative functional clusters are determined using the method of claim 36.

57. The method of claim 55 wherein said putative functional reference clusters are determined using the method of claim 16 and said putative functional clusters are determined using the method of claim 36.

58. The method of claim 52 wherein said putative functional reference clusters are determined using the method of claim 25 and said putative functional clusters are determined using the method of claim 39.

59. The method of claim 55 wherein said putative functional reference clusters are determined using the method of claim 25 and said putative functional clusters are determined using the method of claim 39.

60. A method for determining a continuous SVM score for a putative functional cluster comprising the steps of:
a. identifying at least one validated functional cluster from at least one reference protein;
b. identifying at least one putative functional reference cluster on surface of a least one reference protein using the same method that was used to identify said putative functional cluster;
c. representing each validated functional cluster identified in step a) and each putative functional reference cluster identified in step b) with a functional annotation score of the same form, thereby forming two sets of functional annotation scores;
d. representing said putative functional cluster with a functional annotation score of the same form as the functional annotation scores used to represent the putative functional reference clusters and validated functional clusters in step c);
e. using a support vector machine to determine a hyperplane that divides the two set of functional annotation scores determined in step c); and
f. determining a function that monotonically scales with the distance between the said functional annotation score determined in step d) and the hyperplane determined in step e), thereby determining a continuous SVM score of said putative functional cluster.

61. The method of claim 60 wherein said functional annotation score is a one dimensional functional annotation score selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

62. The method of claim 60 wherein said functional annotation score is a multi-dimensional functional annotation score comprising one subscore selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

63. The method of claim 60 wherein in said functional annotation score is a multi-dimensional functional annotation score comprising:
a. a first subscore that reflects the residue conservation of a putative functional reference cluster, putative functional cluster or a validated functional cluster; and
b. a second subscore that reflects a topographic aspect that may be associated with a concave putative functional reference cluster, putative functional cluster or a validated functional cluster.

64. The methods of claim 63 wherein said first subscore is selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score;
and said second subscore is selected from the group consisting of: the: cluster volume, cluster surface area, cluster “mouth” area, cluster “mouth” circumference, and cluster depth.

65. The method of claim 60 wherein said functional annotation score is a four dimensional functional annotation score consisting of the: cluster maximum residue conservation z-score, cluster surface area, cluster “mouth” area, and cluster depth.

66. The method of claim 62 wherein said putative functional cluster is determined using the method of claim 36 and said putative functional reference clusters are determined using the method of claim 16.

67. The method of claim 65 wherein said putative functional cluster is determined using the method of claim 36 and said putative functional reference clusters are determined using the method of claim 16.

68. The method of claim 62 wherein said putative functional cluster is determined using the method of claim 39 and said putative functional reference clusters are determined using the method of claim 25.

69. The method of claim 65 wherein said putative functional cluster is determined using the method of claim 39 and said putative functional reference clusters are determined using the method of claim 25.

70. A method for determining a continuous SVM score for a putative functional cluster comprising the steps of:
a. identifying at least one validated functional cluster from at least one reference protein;
b. identifying at least one putative functional reference cluster on surface of a least one reference protein using the same method that was used to identify said putative functional cluster;
c. representing each putative validated functional cluster identified in step a) and each putative functional reference cluster identified in step b) with a functional annotation score of the same form, thereby forming two sets of functional annotation scores;
d. representing said putative functional cluster with a functional annotation score of the same form as the functional annotation scores used to represent the putative functional reference clusters and validated functional clusters in step c);
e. using a support vector machine to determine a hyperplane that divides the two set of functional annotation scores determined in step c); and
f. determining the distance between the said functional annotation score determined in step d) and the hyperplane determined in step e), thereby determining a continuous SVM score of said putative functional cluster.

71. The method of claim 70 wherein said functional annotation score is a one dimensional functional annotation score selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

72. The method of claim 70 wherein said functional annotation score is a multi-dimensional functional annotation score comprising one subscore selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

73. The method of claim 70 wherein in said functional annotation score is a multi-dimensional functional annotation score comprising:
a. a first subscore that reflects the residue conservation of a putative functional reference cluster, putative functional cluster or a validated functional cluster; and
b. a second subscore that reflects a topographic aspect that may be associated with a concave putative functional reference cluster, putative functional cluster or a validated functional cluster.

74. The methods of claim 73 wherein said first subscore is selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score; and said second subscore is selected from the group consisting of: the: cluster volume, cluster surface area, cluster “mouth” area, cluster “mouth” circumference, and cluster depth.

75. The method of claim 70 wherein said functional annotation score is a four dimensional functional annotation score consisting of the: cluster maximum residue conservation z-score, cluster surface area, cluster “mouth” area, and cluster depth.

76. The method of claim 72 wherein said putative functional cluster is determined using the method of claim 36 and said putative functional reference clusters are determined using the method of claim 16.

77. The method of claim 75 wherein said putative functional cluster is determined using the method of claim 36 and said putative functional reference clusters are determined using the method of claim 16.

78. The method of claim 72 wherein said putative functional cluster is determined using the method of claim 39 and said putative functional reference clusters are determined using the method of claim 25.

79. The method of claim 75 wherein said putative functional cluster is determined using the method of claim 39 and said putative functional reference clusters are determined using the method of claim 25.

80. A method for determining a continuous SVM score for a putative functional cluster determined using the methods of claim 36 comprising the steps of:
a. identifying at least one validated functional cluster from at least one reference protein;
b. identifying at least one putative functional reference cluster on surface of a least one reference protein using the method of claim 16;
c. representing each putative validated functional cluster identified in step a) and each putative functional reference cluster identified in step b) with a functional annotation score of the same form, thereby forming two sets of functional annotation scores;
d. representing said putative functional cluster with a functional annotation score of the same form as the functional annotation scores used to represent the putative functional reference clusters and validated functional clusters in step c);
e. using a support vector machine to determine a hyperplane that divides the two set of functional annotation scores determined in step c); and
f. determining a continuous SVM score of said putative functional cluster according to Equation 8.

81. The method of claim 80 wherein said functional annotation score is a one dimensional functional annotation score selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

82. The method of claim 80 wherein said functional annotation score is a multi-dimensional functional annotation score comprising one subscore selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

83. The method of claim 80 wherein in said functional annotation score is a multi-dimensional functional annotation score comprising:
a. a first subscore that reflects the residue conservation of a putative functional reference cluster, putative functional cluster or a validated functional cluster; and
b. a second subscore that reflects a topographic aspect that may be associated with a concave putative functional reference cluster, putative functional cluster or a validated functional cluster.

84. The methods of claim 83 wherein said first subscore is selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score;
and said second subscore is selected from the group consisting of: the: cluster volume, cluster surface area, cluster “mouth” area, cluster “mouth” circumference, and cluster depth.

85. The method of claim 80 wherein said functional annotation score is a four dimensional functional annotation score consisting of the: cluster maximum residue conservation z-score, cluster surface area, cluster “mouth” area, and cluster depth.

86. A method for determining the probability that a putative functional cluster is functional comprising the steps of:
a. selecting a plurality of reference proteins, each comprising a validated functional cluster;
b. for each reference protein, identifying one or more reference functional clusters using the same method that was used to identify said putative functional cluster;
c. for each reference functional cluster that was identified in step b), determining a functional annotation score that characterizes it;
d. selecting a lower threshold score of at least 35% and not greater than 100% and an upper threshold score that is not greater than 65%;
e. determining the fraction of reference functional clusters identified in step b) that correctly correspond to validated functional clusters selected in step a) based upon the upper and lower threshold scores selected in step d) at each functional annotation score, for a plurality of functional annotation scores;
f. determining a functional annotation score of the same type as used in step c) that characterizes said putative functional cluster; and
g. identifying the probability that said putative functional cluster is functional with the fraction of reference functional clusters, each characterized by a functional annotation score that is equal to the functional annotation score of said putative functional cluster, that are correctly identified as corresponding to validated functional clusters in step e).

87. The method of claim 86 wherein said functional annotation score is a one dimensional functional annotation score selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

88. The method of claim 86 wherein said functional annotation score is a multi-dimensional functional annotation score comprising one subscore selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score.

89. The method of claim 86 wherein in said functional annotation score is a multi-dimensional functional annotation score comprising:
a. a first subscore that reflects the residue conservation of a putative functional reference cluster, putative functional cluster or a validated functional cluster; and
b. a second subscore that reflects a topographic aspect that may be associated with a concave putative functional reference cluster, putative functional cluster or a validated functional cluster.

90. The methods of claim 89 wherein said first, subscore is selected from the group consisting of the: cluster maximum residue conservation z-score, cluster averaged residue conservation z-score, cluster median residue conservation z-score, cluster maximum neighbor averaged residue conservation z-score, cluster averaged neighbor averaged residue conservation z-score, cluster median neighbor averaged residue conservation z-score, cluster maximum residue conservation p-score, cluster averaged residue conservation p-score, cluster median residue conservation p-score, cluster maximum neighbor averaged residue conservation p-score, cluster averaged neighbor averaged residue conservation p-score, and cluster median neighbor averaged residue conservation p-score;
and said second subscore is selected from the group consisting of: the: cluster volume, cluster surface area, cluster “mouth” area, cluster “mouth” circumference, and cluster depth.

91. The method of claim 86 wherein said functional annotation score is a four dimensional functional annotation score consisting of the: cluster maximum residue conservation z-score, cluster surface area, cluster “mouth” area, and cluster depth.

92. The method of claim 86 wherein said functional annotation score is a continuous SVM score determined using the method of claim 64.

93. The method of claim 86 wherein said functional annotation score is a continuous SVM score determined using the method of claim 74.

94. The method of claim 84 wherein said functional annotation score is a continuous SVM score determined using the method of claim 84.

95. A method for determining the probability that a putative functional cluster determined using the method of claim 36 is functional comprising the steps of:
a. selecting a plurality of reference proteins, each comprising a validated functional cluster;
b. for each reference protein, identifying one or more reference functional clusters using the method of claim 16;
c. for each reference functional cluster that was identified in step b), determining a functional annotation score that characterizes it;
d. selecting a lower threshold score of at least 35% and not greater than 100% and an upper threshold score that is not greater than 65%;
e. determining the fraction of reference functional clusters identified in step b) that correctly correspond to validated functional clusters selected in step a) based upon the upper and lower threshold scores selected in step d) at each functional annotation score, for a plurality of functional annotation scores;
f. determining a functional annotation score of the same type as used in step c) that characterizes said putative functional cluster; and
g. identifying the probability that the putative functional cluster is functional with the fraction of reference functional clusters, each characterized by a functional annotation score that is equal to the functional annotation score of said putative functional cluster, that are correctly identified as corresponding to validated functional clusters in step e).

96. The method of claim 95 wherein said functional annotation score is a continuous SVM score determined using the method of claim 66.

97. The method of claim 95 wherein said functional annotation score is a continuous SVM score determined using the method of claim 67.

98. The method of claim 95 wherein said functional annotation score is a continuous SVM score determined using the method of claim 76.

99. The method of claim 95 wherein said functional annotation score is a continuous SVM score determined using the method of claim 77.

100. The method of claim 95 wherein said functional annotation score is a continuous SVM score determined using the method of claim 82.

101. A computer system comprising:
a. a processor;
b. a memory;
c. programming for an operating system; and
d. programming for the method of claim 16.

102. A computer system comprising:
a. a processor;
b. a memory;
c. programming for an operating system; and
d. programming for the method of claim 36.

103. A computer system comprising:
a. a processor;
b. a memory;
c. programming for an operating system; and
d. programming for the method of claim 49.

104. A computer system comprising:
a. a processor;
b. a memory;
c. programming for an operating system; and
d. programming for the method of claim 57.

105. A computer system comprising:
a. a processor;
b. a memory;
c. programming for an operating system; and
d. programming for the method of claim 67.

106. A computer system comprising:
a. a processor;
b. a memory;
c. programming for an operating system; and
d. programming for the method of claim 77.

107. A computer system comprising:
a. a processor;
b. a memory;
c. programming for an operating system; and
d. programming for the method of claim 85.

108. A computer system comprising:
a. a processor;
b. a memory;
c. programming for an operating system; and
d. programming for the method of claim 91.

109. A computer system comprising:
a. a processor;
b. a memory;
c. programming for an operating system; and
d. programming for the method of claim 101.


SEQUENCE LISTING (29 sequences)

SEQ ID NO: 1 - protein, 327 aa, Escherichia coli
AYIAKQRQISFVKSHFSRQLEERLGLIEVQAPILSRVGDGTQDNLSGAEKAVQVKVKALPDAQFEVVHSLAKWKRQTLGQHDFSAGEGLYTHMKALRPDEDRLSPLHSVYVDQWDWERVMGDGERQFSTLKSTVEAIWAGIKATEAAVSEEFGLAPFLPDQIHFVHSQELLSRYPDLDAKGRERAIAKDLGAVFLVGIGGKLSDGHRHDVRAPDYDDWSTPSELGHAGLNGDILVWNPVLEDAFELSSMGIRVDADTLKHQLALTGDEDRLELEWHQALLRGEMPQTIGGGIGQSRLTMLLLQLPHIGQVQAGVWPAAVRESVPSLL

SEQ ID NO: 2 - protein, 327 aa, Shigella flexneri
AYIAKQRQISFVKSHFSRQLEERLGLIEVQAPILSRVGDGTQDNLSGCEKAVQVKVKALPDAQFEVVHSLAKWKRQTLGQHDFSAGEGLYTHMKALRPDEDRLSPLHSVYVDQWDWERVMGDGERQLSTLKSTVEAIWAGIKATEAAVNEEFGLAPFLPDQIHFVHSQELLSRYPDLDAKGRERAIAKDLGAVFLVGIGGKLSDGHRHDVRAPDYDDWSTPSELGHAGLNGDILVWNPVLEDAFELSSMGIRVDADTLKHQLALTGDEDRLQLEWHQALLRGEMPQTIGGGIGQSRLTMLLLQLPHIGQVQCGVWPAAVRESVPSLL

SEQ ID NO: 3 - protein, 327 aa, Salmonella enterica
AYIAKQRQISFVKSHFSRQLEERLGLIEVQAPILSRVGDGTQDNLSGCEKAVQVKVKALPDAQFEVVHSLAKWKRQTLGQHDFSAGEGLYTHMKALRPDEDRLSPLHSVYVDQWDWERVMGDGERQFSTLKSTVEAIWAGIKATEAEVHKQFGLAPFLPDQIHFVHSQELLARFPDLDAKGRERAIAKELGAVFLVGIGGKLSDGRRHDVRAPDYDDWSSASELGYAGLNGDILVWNPVLEDAFELSSMGIRVDADTLMRQLALTGDEDRLQLEWHQALLRGEMPQTIGGGIGQSRLTMLLLQLPHIGQVQCGVWPAQVRESIPAIL

SEQ ID NO: 4 - protein, 327 aa, Salmonella typhimurium
AYIAKQRQISFVKSHFSRQLEERLGLIEVQAPILSRVGDGTQDNLSGCEKAVQVKVKALPDAQFEVVHSLAKWKRQTLGQHDFSAGEGLYTHMKALRPDEDRLSPLHSVYVDQWDWERVMGDGERQFSTLKSTVEAIWAGIKATEAEVHKQFGLAPFLPEQIQFVHSQELLARFPDLDAKGRERAIAKELGAVFLVGIGGKLSDGHRHDVRAPDYDDWSSASELGYAGLNGDILVWNPVLEDAFELSSMGIRVDADTLMRQLALTGDEDRLQLEWHQALLRGEMPQTIGGGIGQSRLTMLLLQLPHIGQVQCGVWPAQVRESIPAIL

SEQ ID NO: 5 - protein, 327 aa, Yersinia pestis
QFIQKQQQISFVKSFFSRQLEQQLGLIEVQAPILSRVGDGTQDNLSGSEKAVQVKVKSLPDSTFEVVHSLAKWKRKTLGRFDFGADQGVYTHMKALRPDEDRLSAIHSVYVDQWDWERVMGDGERNLAYLKSTVNKIYAAIKETEAAISAEFGVKPFLPDHIQFIHSESLRARFPDLDAKGRERAIAKELGAVFLIGIGGKLADGQSHDVRAPDYDDWTSPSAEGFSGLNGDIIVWNPILEDAFEISSMGIRVDAEALKRQLALTGDEDRLELEWHQSLLRGEMPQTIGGGIGQSRLVMLLLQKQHIGQVQCGVWGPEISEKVDGLL

SEQ ID NO: 6 - protein, 327 aa, Streptococcus agalactiae
SFIHQQQEISFVKNTFTQYLIDKLEIVEVQGPILSQVGDGMQDNLSGIEHPVSVKVLNIPEAEFEVVHSLAKWKRHTLARFGFNEGEGLFVHMKALRPDEDSLDPTHSVYVDQWDWEKVIPDGRRNLDYLKETVEKIYKAIRLTELAVEARFDIESILPKRITFIHTEELVEKYPDLSPKERENAIAKEYGAVFLIGIGGELADGKPHDGRAPDYDDWTTPSENGFKGLNGDILVWNEQLGTAFELSSMGIRVDEDALKRQVVLTGDEDRLEFEWHKTLLRGFFPLTIGGGIGQSRLAMFLLRKKHIGEVQSSVWPKEVRDTFENIL

SEQ ID NO: 7 - protein, 327 aa, Streptococcus agalactiae
SFIHQQQEISFVKNTFTQYLIDKLEIVEVQGPILSQVGDGMQDNLSGIEHPVSVKVLNIPEAEFEVVHSLAKWKRHTLARFGFNEGEGLFVHMKALRPDEDSLDPTHSVYVDQWDWEKVIPDGRRNLDYLKETVEKIYKAIRLTELAVEARFDIESILPKRITFIHTEELVEKYPDLSPKERENAIAKEYGAVFLIGIGGELADGKPHDGRAPDYDDWTTPSENGFKGLNGDILVWNEQLGTAFELSSMGIRVDEDALKRQVVLTGDEGRLEFEWHKTLLRGFFPLTIGGGIGQSRLAMFLLRKKHIGEVQSSVWPKEVRDTFENIL

SEQ ID NO: 8 - protein, 327 aa, Streptococcus pneumoniae
SFIHQQEEISFVKNTFTQYLKDKLEVVEVQGPILSKVGDGMQDNLSGVENPVSVKVLQIPDATYEVVHSLAKWKRHTLARFGFGEGEGLFVHMKALRPDEDSLDATHSVYVDQWDWEKVIPNGKRNIVYLKETVEKIYKAIRLTELAVEARYDIESILPKQITFIHTEELVERYPDLTPKERENAICKEFGAVFLIGIGGELPDGKPHDGRAPDYDDWTSESENGYKGLNGDILVWNESLGGAFELSSMGIRVDEETLRRQVEITGDEDRLELEWHKSLLNGLFPLTIGGGIGQSRMAMFLLRKRHIGEVQTSVWPQEVRDTYENIL

SEQ ID NO: 9 - protein, 327 aa, Streptococcus pneumoniae
SFIHQQEEISFVKNTFTQYLKDKLEVVEVQGPILSKVGDGMQDNLSGVENPVSVKVLQIPDATYEVVHSLAKWKRHTLARFGFGEGEGLFVHMKALRPDEDSLDATHSVYVDQWDWEKVIPNGKRNIVYLKETVEKIYKAIRLTELAVEARYDIESILPKQITFIHTEELVERYPDLTSKERENAICKEFGAVFLIGIGGELPDGKPHDGRAPDYDDWTSESENGYKGLNGDILVWNESLGGAFELSSMGIRVDEETLRRQVEITGDEDRLELEWHKSLLNGLFPLTIGGGIGQSRMAMFLLRKRHIGEVQTSVWPQEVRDTYENIL

SEQ ID NO: 10 - protein, 327 aa, Streptococcus pyogenes MGAS8232
SFIHQQEEISFVKNTFTQYLIAKLDVVEVQGPILSRVGDGMQDNLSGTENPVSVNVLKIPNATFEVVHSLAKWKRHTLARFGFNEGEGLVVNMKALRPDEDSLDQTHSVYVDQWDWEKVIPDGKRNLAYLKETVETIYKVIRLTELAVEARYDIEAVLPKKITFIHTEELVAKYPDLTPKERENAITKEFGAVFLIGIGGVLPDGKPHDGRAPDYDDWTTETENGYHGLNGDILVWNDQLGSAFELSSMGIRVDEEALKRQVEMTGDQDRLAFDWHKSLLNGLFPLTIGGGIGQSRMVMFLLRKKHIGEVQTSVWPQEVRDSYDNIL

SEQ ID NO: 11 - protein, 327 aa, Streptococcus pyogenes M1 GAS
SFIHQQEEISFVKNTFTQYLIAKLDVVEVQGPILSRVGDGMQDNLSGTENPVSVNVLKIPNATFEVVHSLAKWKRHTLARFGFNEGEGLVVNMKALRPDEDSLDQTHSVYVDQWDWEKVIPDGKRNLAYLKETVETIYKVIRLTELAVEARYDIEAVLPKKITFIHTEELVAKYPDLTPKERENAITKEFGAVFLIGIGGVLPDGKPHDGRAPDYDDWTTETENGYHGLNGDILVWNDQLGSAFELSSMGIRVDEEALKRQVEMTGDQDRLGFDWHKSLLNGLFPLTIGGGIGQSRMVMFLLRKQHIGEVQTSVWPQEVRDSYDNIL

SEQ ID NO: 12 - protein, 327 aa, Haemophilus influenzae Rd KW20
TFILQQQEISFVKNTFTQNLIEQLGIIEVQGPILSQVGNGMQDNLSGIEKAVQVNVKCIPNAVFEVVHSLAKWKRHTLARFNFKEDEGLFVHMKALRPDEDSLDPTHSVYVDQWDWEKVIPEGRRNFAYLKETVNSIYRAIRLTELAVEARFDIPSILPKQITFVHSEDLVKRYPDLSSKERENAICKEYGAVFLIGIGGKLSDGKPHDGRAPDYDDWTTESENGYKGLNGDILVWNDQLGKAFELSSMGIRVDESALRLQVGLTGDEDHLKMDWHQDLLNGKLPLTIGGGIGQSRLAMLLLRKKHIGEVQSSVWPKEMLEEFSNIL

SEQ ID NO: 13 - protein, 327 aa, Pasteurella multocida subsp. multocida str. Pm70
SFILQQQEISFAKNTFTEKLAEHLGIVEVQGPILSQVGNGIQDNLSGAEKAVQVNVKQITDATFEVVHSLAKWKRHTLARFNFAQGEGLFVHMTALRPDEDSLDQTHSVYVDQWDWEKVISAEQRNLAYLKETVRAIYAAILETEEAVSKKFGLATFLPKDIQFVHSEELVQRFPNMNDKERENAICKEYGAVFLIGIGGKLSDGKPHDVRAPDYDDWTTPSEGEYKGLNGDILVWNPILERAFELSSMGIRVDETALRKQLALTNNEDRLKFDWHQDLVNGRLPLSIGGGIGRSRLVMLLLQKKHIGEVQSSVWPKWVMEQFDNIL

SEQ ID NO: 14 - protein, 327 aa, Haemophilus somnus 129PT
SFILQQQEISFAKNTFTEKLIEHLGIIEVQGPILSQVGNGIQDNLSGTEKAVQVNVKQITDAKFEVVHSLAKWKRHTLARFNFAENEGLFVHMKALRPDEDSLDQTHSVYVDQWDWEKVIPTGRRNLAYLKETVRSIYQAILETEDAVHQKFGLSKFLPREITFIHSEELVQRYPELNDKQRENAICKEYGAVFLIGIGGVLSDGKPHDKRAPDYDDWTTPSEGEYLGLNGDILVWNPVLERAFEVSSMGIRVDETALRKQLALTGDEDRLQFDWHQDLVNGRLPLSIGGGIGQSRLAMLLLQKKHIGEVQSSVWPKVVTEQFENIL

SEQ ID NO: 15 - protein, 327 aa, Treponema pallidum subsp. pallidum str. Nichols
SFILQQQGISFAKHTFTQKLMEHLGLIEVQGPLLSQVGDGIQDGLSGREKAVSVSVKQIPGTAFEVVHSLAKWKRHTLARYGFQDNEGLFVHMIALRPDEDFLDQVRSVCVDQWDWEKVVPVGSRNLAYLKDTVRKVYGALRESEVLVSERFGLRAFLPADIVFVQSEELVRRYPHLDSKGREDAICKEHGAVFLIGIGGVLSDGKPHDVRAPDYDDWTTPSEGEYKGLNGDILVWNPVLGRAFEVSSMGIRVDEGALRTQLALTGDEDSLACSWHQDLINGRLPQSIGGGIGQSRLAMLLLQRKHIGEVQASVWPRSVREEFENIL

SEQ ID NO: 16 - protein, 308 aa, Bacillus cereus ATCC 14579
QIAIKEVKTFFEDQLAKRLELFRVSAPLFVTKKSGLNDHLNGVERPIEFDMLHSGEELEIVHSLAKWKRFALHEYGYEAGEGLYTNMNAIRRDEELDATHSIYVDQWDWEKIVQKEWRTVEYLQKTVQTIYGIFKDLEDHLFEKYPFLGKYLPEEIVFVTSQELEDKYPELTPKDREHAIAKEHGAVFIIGIGDALRSGEKHDGRAADYDDWKLNGDILFWHPVLQSSFELSSMGIRVDSKSLDEQLTKTGEDFKREYDFHKGILEDVLPLTIGGGIGQSRMCMYFLRKAHIGEVQSSVWPDDLREAC

SEQ ID NO: 17 - protein, 308 aa, Bacillus anthracis str. A2012
QIAIKEVKTFFEDQLAKRLELFRVSAPLFVTKKSGLNDHLNGVERPIEFDMLHSGEELEIVHSLAKWKRFALHEYGYEAGEGLYTNMNAIRRDEELDATHSIYVDQWDWEKIVQKEWRTVDYLQKTVLTIYGIFKDLEDHLFEKYPFLGKYLPEEIVFVTSQELEDKYPELTPKDREHAIAKEHGAVFIIGIGDALRSGEKHDGRAADYDDWKLNGDILFWHPVLQSSFELSSMGIRVDSKSLDEQLTKTGEDFKREYDFHKGILEDVLPLTIGGGIGQSRMCMYFLRKAHIGEVQSSVWPDDLREAC

SEQ ID NO: 18 - protein, 309 aa, Clostridium tetani E88
AIKEVKDCFERALAKQLNLIRVSAPLFVRCDKGLNDNLNGVERPVKFTVKDDNEAAVEIVHSLAKWKRMALYRYNFNADEGLYTDMNAIRRDEELDNTHSIYVDQWDWERIIKKEDRNEEYLKDIVRKIFKAFKETEEHINKLYPFLGEVLPEEVFFMTTQELEDMFPDLTAKEREDAITKEKKAVFLMKIGKTLESGEKHDGRAPDYDDWELNGDILFWNPVLNKAFELSSMGIRVDEESLLKQLKLANCEERKELQFHKMLLEKKLPYTIGGGIGQSRMCMLFLKKAHIGEVQSSIWPEEMIKFCEE

SEQ ID NO: 19 - protein, 161 aa, Clostridium thermocellum ATCC 27405
DMNAIRRDEDLDNLHSIYVDQWDWELVINKEDRNEETLKNIVKKIYNVLKKTEDFIAEQYPQIPKFLPEDIFFITTQELEDMYPELSPKEREDAIAKEKKAIFLMKIGGVLKSGKKHDGRAPDYDDWTLNGDIILWYPLLERSFEISSMGIRVDENSLLSQ

SEQ ID NO: 20 - protein, 307 aa, Fusobacterium nucleatum subsp. vincentii ATCC 49256
EIAIKKVKDFFESRLAKELDLLRVSAPLFVIPESGLNDNLNGTERPVSFDTKSGERVEIVHSLAKWKRMALYRYNIENHKGIYTDMNAIRRDEDTDFIHSYYVDQWDWEKIISKEDRNEEYLKETVRKIYCVFKETEEYITTEYPKLTKKLPEEITFITSQELENKYPNLTPKNREHAAAKEYGAIFLMKIGGKLSSGEKHDGRAPDYDDWDLNGDIIFNYPLLGIGLELSSMGIRVDEKSLDEQLKIANCEDRRSLPYHQMILNKVLPYTIGGGIGQSRICMFFLDKLHIGEVQASIWSQEVHEIC

SEQ ID NO: 21 - protein, 307 aa, Fusobacterium nucleatum subsp. nucleatum ATCC 25586
EIAIKKVKDFFESHLSKELDLLRVSAPLFVIPESGLNDNLNGTERPVSFDTKSGERVEIVHSLAKWKRMALYRYNIENDKGIYTDMNAIRRDEDTDFIHSYYVDQWDWEKIISKEDRNEEYLKDVVRKIYSVFKKTEEYITTEYPKLTKKLPEEITFITAQELENKYPNLTPKNREHAAAKEYGAIFLMKIGGKLSSGEKHDGRAPDYDDWDLNGDIIFNYPLLGIGLELSSMGIRVDEKSLDEQLKIANCEDRRSLPYHQMILNKVLPYTIGGGIGQSRICMFFLDKLHIGEVQASIWSQEVHEIC

SEQ ID NO: 22 - protein, 309 aa, Clostridium perfringens str. 13
AIKELKDFFEDSLAKNLNLTRVSAPLFVNKGSGLNDDLNGIERPVSFDMKAMPEFNIQIVHSLAKWKRLALHRYEFEHGEGLYTDMNAIRRDEDLDNIHSIYVDQWDWEKIIDKEERNLETLKETVKSIYGTFKATEDFIVAKYPHIEKILPEDITFITSQELEDRYPDLTSKERETAICKEFGAVFIIGIGGKLASGEKHDDRSPDYDDWTLNGDLLFYYPLFDEAVELSSMGIRVDEESLLKQLKIAECEERKELPFHQMLLEGKLPYTIGGGIGQSRICMFFLRKAHIGEVQASMWDEDMIRTCEE

SEQ ID NO: 23 - protein, 309 aa, Lactobacillus gasseri
AIVFIRETFQDKIAEKLNVQRMSAPMFVEKSTGLNDNLNGVERPVSFDMKAMPNDTIEVVHSLAKWKRLALKRYGFGMHEGLYTNMNAIRRDEDLDNFHSIYVDQWDWEKIISKDERNIDTLKDTVKQIFKAIKETEKEVAARYPSSTYRLPNEITFITTQELEDRWPDLTPDEREDKIAKEKKAVFLMKIGDKLKRSGKPHDGRAPDYDDWQLNGDLLFWYEPLQRKLEISSMGIRVSEESLKTQLKKAHAEERAALPFHKMLLNGELPYTIGGGIGQSRLCMLLLGKAHIGEVQASIWPPKMIEECE

SEQ ID NO: 24 - protein, 301 aa, Lactobacillus plantarum WCFS1
QQAIRYIRETFQDEFGKQLNLSRLSAPMFVEKKTGLNDNLNGVEKPVSFTMQDMGDEQIEIVHSLAKWKRVALKRYGFDMHEGLYTNMNAIRKDEDLDNYHSAYVDQWDWEKVISKEERTVETLKAAVRQIFKVIKHMEHEVWYKFPQAVHHLPDEIHFLTTQELEDMYPDMTPRERENAICKKLGCVFLMQIGWKLDSGERHDGRAPDYDDWKLNGDILFWYEPLDQAIEISSMGIRVDAESMKKQLKDVDAEDRLSLPYHQMILNADVPYTIGGGIGQSRLCMLLLGKAHVGEVQAALW

SEQ ID NO: 25 - protein, 314 aa, Lactobacillus delbrueckii subsp. bulgaricus
IAKRKRQSATSGKPSSEFGTAMNLERISAPMFVKKSSGLNDNLSGWEKPVSFTLHDGNEGELQIVHSLAKWKRWALKHYGFSHGEGLFTNMNAIRKDEEVLDNLHSVYVDQWDWEKVIDKSERTEATLRQTVQRIFETIKGMEYHVRALYPQAAYHLPEEISFVTSEELEARWPSLTPSEREDKICQEKGAVFLEHIGGALPLSKKPHDLRAPDYDDWTLNGDLLFWYEPLQRAFEVSSMSIRVDEDRLQEQLKLAGAEDRLDLPFHQALLKGDLPYSIGGGIGQSRLCMLLLQGPHGEVQASIWPDEIVEKCQ

SEQ ID NO: 26 - protein, 325 aa, Bacteroides thetaiotaomicron VPI-5482
LKQTELGIKQIKEFFQLNLSSELRLRRVTAPLFVLKGMGINDDLNGIERPVSFPIKDLGDAQAEVVHSLAKWKRLTLADYNIEPGYGIYTDMNAIRSDEELGNLHSLYVDQWDWERVITNEDRNVEFLKEIVNRIYAAMIRTEYMVYEMYPQIKPCLPQKLHFIHSEELRQLYPNLEPKCREHAICQKYGAVFIIGIGCKLSDGKKHDGRAPDYDDYTSTGLNNLPGLNGDLLLWDDVLQRSIELSSMGVRVDREALQRQLKEENEEERLKLYFHKRLMDDTLPLSIGGGIGQSRLCMFYLRKAHIGEIQASIWPEDMRKECEEL

SEQ ID NO: 27 - protein, 315 aa, Mycoplasma penetrans HF-2
SILETQKAIKFIKDLFQVNLAHALKLHRVTAPLVLERNKGINDDLNGSENPVTFTSDGNGISGEIPQSLAKWKRMMLGKYEIPLHEGIYADMNAIRKDESLSSIHSIYVDQWDWELHIKKTERNLETLKVVVKKIYEIIRLCQKEVNKKYEWFAENLLPEEITFISSEDLLQRYPNKTPKERERLIASKYKAVFIIGIGDNLSDGKPHDLRAPDYDDWKLNGDIIVWNETTKSALELSSMGIRVDEVSLVEQLDKSNNNSRKELDFHKKLINKEFPYSIGGGIGQSRLCYFLLHKQHIGEVQSSLWPKDILEEAE

SEQ ID NO: 28 - protein, 72 aa, Mycoplasma gallisepticum
IRVNKEVLLKQLEESKQNERLELMFHKKLVNGELHQTIGGGIGQSRLCYFLLQKDHIGEVQASHWSDEIVAE

SEQ ID NO: 29 - protein, 309 aa, Ureaplasma parvum serovar 3 str. ATCC 700970
QKAIVEIKNSFQKHFAKNLNLSRVTAPLFVEGQSGLNDHLDHKQKAVSFYAKKLDKTLEIVQSLAKWKRLALLDYGFSLYEGLYTDMNAIRADDDIDEIHSIYVDQWDWEILINNQDCNLDFLKSIVNKIYSTIRIVQLEIDQLYNPKQIILPDSITFISSQELEDLYPHLSPSRREYEFAKIHKAIFIYQIGYPLKSGYIQSIRSPEYDNWNLNGDLIVYHKLNDQAIELSSMGIRVSKQDFIKQTNFANLKNDQENNFYHQMILNNQLPQTIGGGIGQSRLCMFLLNKKHIGEVQVSVWPNEYKDEL